(19) United States
(12) Patent Application Publication
(10) Pub. No.: US 2011/0014117 A1
(43) Pub. Date: Jan. 20, 2011

(54) ANTI-IGF1R

(75) Inventors: Yan Wang, Warren, NJ (US); Jonathan A. Pachter, East Setauket, NY (US); Judith Anne Hailey, East Windsor, NJ (US); Peter Brams, Sacramento, CA (US); Denise Williams, Livermore, CA (US); Mohan Srinivasan, San Jose, CA (US); Mary Diane Feingersh, Hayward, CA (US)

Correspondence Address: MERCK;PATENT DEPARTMENT (K-6-1, 1990), 2000 GALLOPING HILL ROAD, KENILWORTH, NJ, 07033-0530, US

(73) Assignee: Schering Corporation

(21) Appl. No.: 12/663,651

(22) PCT Filed: Jun. 25, 2008
(86) PCT No.: PCT/US08/07920

§ 371 Date: Sep. 24, 2010

Related U.S. Application Data
(60) Provisional application No. 60/946,803, filed on Jun. 28, 2007.

(51) Int. Cl. A61K 51/00; C07K 16/00; A61K 39/395; C07H 21/04; C12N 15/63; C12N 5/10; C12N 1/20; A61P 35/00; A61P 35/04; A61K 49/00; C12N 5/12; C12P 21/04; A61K 9/127

(52) U.S. Cl. 424/1.11; 530/387.1; 530/388.1; 530/387.3; 530/387.2; 424/130.1; 536/23.1; 435/320.1; 435/326; 435/252.3; 435/252.33; 424/141.1; 424/9.1; 435/346; 424/142.1; 435/70.2; 424/450; 424/133.1

(57) ABSTRACT

The present invention relates in part to anti-IGF1R antibodies and antigen-binding compositions thereof along with methods of use thereof. For example, methods of treating medical disorders such as cancer are covered.

The present application claims the benefit of U.S. provisional patent application No. 60/946,803; filed Jun. 28, 2007, which is herein incorporated by reference in its entirety.

### FIELD OF THE INVENTION

The present invention relates, in part, to therapeutic antibodies and methods of use thereof.

### BACKGROUND OF THE INVENTION

The insulin-like growth factors, also known as somatomedins, include insulin-like growth factor-I (IGF-I) and insulin-like growth factor-II (IGF-II) (Klapper, et al., (1983) Endocrinol. 112:2215 and Rinderknecht, et al., (1978) Febs. Lett. 89:283). These growth factors exert mitogenic activity on various cell types, including tumor cells (Macaulay, (1992) Br. J. Cancer 65:311), by binding to a common receptor named the insulin-like growth factor receptor-1 (IGF1R) (Sepp-Lorenzino, (1998) Breast Cancer Research and Treatment 47:235). Interaction of IGFs with IGF1R activates the receptor by triggering autophosphorylation of the receptor on tyrosine residues (Butler, et al., (1998) Comparative Biochemistry and Physiology 121:19). Once activated, IGF1R, in turn, phosphorylates intracellular targets to activate cellular signaling pathways. This receptor activation is critical for stimulation of tumor cell growth and survival. Therefore, inhibition of IGF1R activity represents a valuable potential method to treat or prevent growth of human cancers and other proliferative diseases.

Several lines of evidence indicate that IGF-I, IGF-II and their receptor IGF1R are important mediators of the malignant phenotype. Plasma levels of IGF-I have been found to be the strongest predictor of prostate cancer risk (Chan, et al., (1998) Science 279:563) and similar epidemiological studies strongly link plasma IGF-I levels with breast, colon and lung cancer risk.

Overexpression of Insulin-like Growth Factor Receptor-1 has also been demonstrated in several cancer cell lines and tumor tissues. IGF1R is overexpressed in 40% of all breast cancer cell lines (Pandini, et al., (1999) Cancer Res. 5:1935) and in 15% of lung cancer cell lines. In breast cancer tumor tissue, IGF1R is overexpressed 6-14 fold and IGF1R exhibits 2-4 fold higher kinase activity as compared to normal tissue (Webster, et al., (1996) Cancer Res. 56:2781 and Pekonen, et al., (1998) Cancer Res. 48:1343). Ninety percent of colorectal cancer tissue biopsies exhibit elevated IGF1R levels wherein the extent of IGF1R expression is correlated with the severity of the disease. Analysis of primary cervical cancer cell cultures and cervical cancer cell lines revealed 3- and 5-fold overexpression of IGF1R, respectively, as compared to normal ectocervical cells (Steller, et al., (1996) Cancer Res. 56:1762). Expression of IGF1R in synovial sarcoma cells also correlated with an aggressive phenotype (i.e., metastasis and high rate of proliferation; Xie, et al., (1999) Cancer Res. 59:3588). Furthermore, acromegaly, a slowly developing disease, is caused by hypersecretion of growth hormone and IGF-I (Ben-Schlomo, et al., (2001) Endocrin. Metab. Clin. North. Am. 30:565-583). Antagonism of IGF1R function may be helpful in treating the disease. There remains a need in the art for IGF1R antagonist therapies for treating or preventing such disease and disorders. Of particular utility are anti-IGF1R antibody based therapies.

### SUMMARY OF THE INVENTION

The present invention provides an isolated antibody or antigen-binding fragment thereof that binds specifically to IGF1R comprising one or more members selected from the group consisting of: (a) CDR-H1, CDR-H2 and CDR-H3 of the variable region of antibody 2C6; (b) CDR-H1, CDR-H2 and CDR-H3 of the variable region of antibody 9H2; (c) CDR-L1, CDR-L2 and CDR-L3 of the variable region of antibody 2C6; and (d) CDR-L1, CDR-L2 and CDR-L3 of the variable region of antibody 9H2 (e.g., as defined by the Kabat or Chothia methods as discussed herein). In an embodiment of the invention, the antibody is a monoclonal antibody. In an embodiment of the invention, the antibody is polyclonal, recombinant, humanized, bispecific, anti-idiotypic, chimeric or labeled. In an embodiment of the invention, the fragment is a single-chain variable fragment, an Fv fragment, a Fab fragment, Fab′ fragment, a F(ab′)₂ fragment, a disulfide-linked Fv. In an embodiment of the invention, the antibody or fragment is linked to a constant region such as a κ light chain, a γ1 heavy chain, a γ2 heavy chain, a γ3 heavy chain or a γ4 heavy chain. The present invention also provides a pharmaceutical composition comprising the antibody or fragment in association with a pharmaceutically acceptable carrier. The present invention also provides a composition comprising the antibody or fragment in association with a further chemotherapeutic agent such as pemetrexed, erlotinib, dasatanib, nilotinib, decatanib, panitumumab, amrubicin, oregovomab, Lep-etu, nolatrexed, azd2171, batabulin, ofatumumab, zanolimumab, edotecarin, tetrandrine, rubitecan, tesmilifene, oblimersen, ticilimumab, ipilimumab, gossypol, Bio 111, 131-I-TM-601, ALT-110, BIO 140, CC 8490, cilengitide, gimatecan, IL13-PE38QQR, INO 1001, IPdR, KRX-0402, lucanthone, LY 317615, neuradiab, vitespan, Rta 744, Sdx 102, talampanel, atrasentan, Xr 311, romidepsin, ADS-100380,

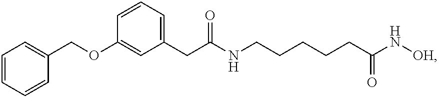

### CG-781, CG-1521

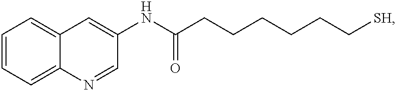

SB-556629, chlamydocin, JNJ-16241199,

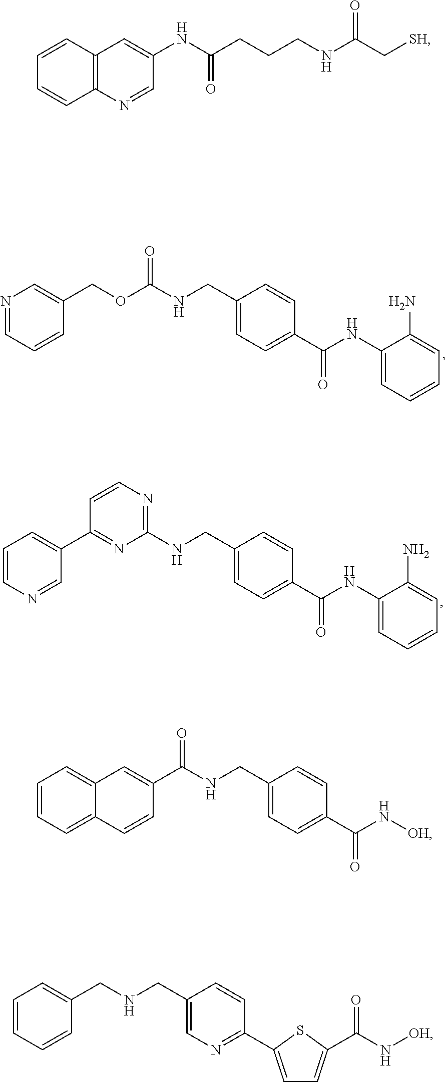

vorinostat, etoposide, gemcitabine, doxorubicin, liposomal doxorubicin, 5′-deoxy-5-fluorouridine, vincristine, temozolomide, ZK-304709, seliciclib; PD0325901, AZD-6244, capecitabine, L-Glutamic acid, N-[4-[2-(2-amino-4,7-dihydro-4-oxo-1H-pyrrolo[2,3-d]pyrimidin-5-yl)ethyl]benzoyl]-, disodium salt, heptahydrate, camptothecin, irinotecan; a combination of irinotecan, 5-fluorouracil and leucovorin; PEG-labeled irinotecan, FOLFOX regimen, tamoxifen, toremifene citrate, anastrazole, exemestane, letrozole, DES(diethylstilbestrol), estradiol, estrogen, conjugated estrogen, bevacizumab, IMC-1C11, CHIR-258,

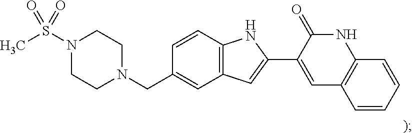

3-[5-(methylsulfonylpiperadinemethyl)-indolyl]-quinolone, vatalanib, AG-013736, AVE-0005, the acetate salt of [D-Ser(Bu t)6, Azgly 10] (pyro-Glu-His-Trp-Ser-Tyr-D-Ser(Bu t)-Leu-Arg-Pro-Azgly-NH2 acetate [C₅₉H84N₁₈O₁₄.(C₂H4O₂)ₓ where x=1 to 2.4], goserelin acetate, leuprolide acetate, triptorelin pamoate, sunitinib, sunitinib malate, medroxyprogesterone acetate, hydroxyprogesterone caproate, megestrol acetate, raloxifene, bicalutamide, flutamide, nilutamide, megestrol acetate, CP-724714; TAK-165, HKI-272, erlotinib, lapatanib, canertinib, ABX-EGF antibody, erbitux, EKB-569, PKI-166, GW-572016, lonafarnib,

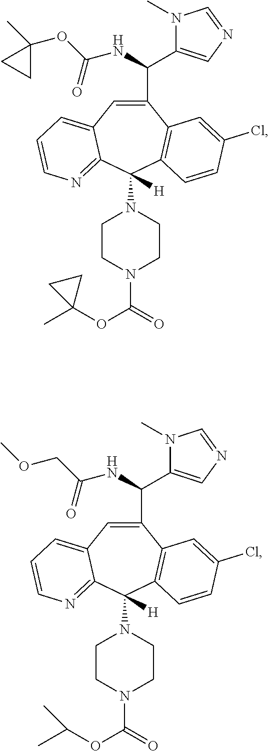

BMS-214662, tipifarnib; amifostine, NVP-LAQ824, suberoyl analide hydroxamic acid, valproic acid, trichostatin A, FK-228, SU11248, sorafenib, KRN951, aminoglutethimide, amsacrine, anagrelide, L-asparaginase, Bacillus Calmette-Guerin (BCG) vaccine, bleomycin, buserelin, busulfan, carboplatin, carmustine, chlorambucil, cisplatin, cladribine, clodronate, cyclophosphamide, cyproterone, cytarabine, dacarbazine, dactinomycin, daunorubicin, diethylstilbestrol, epirubicin, fludarabine, fludrocortisone, fluoxymesterone, flutamide, hydroxyurea, idarubicin, ifosfamide, imatinib, leucovorin, leuprolide, levamisole, lomustine, mechlorethamine, melphalan, 6-mercaptopurine, mesna, methotrexate, mitomycin, mitotane, mitoxantrone, nilutamide, octreotide, oxaliplatin, pamidronate, pentostatin, plicamycin, porfimer, procarbazine, raltitrexed, rituximab, streptozocin, teniposide, testosterone, thalidomide, thioguanine, thiotepa, tretinoin, vindesine, 13-cis-retinoic acid, phenylalanine mustard, uracil mustard, estramustine, altretamine, floxuridine, 5-deooxyuridine, cytosine arabinoside, 6-mercaptopurine, deoxycoformycin, calcitriol, valrubicin, mithramycin, vinblastine, vinorelbine, topotecan, razoxin, marimastat, COL-3, neovastat, BMS-275291, squalamine, endostatin, SU5416, SU6668, EMD121974, interleukin-12, IM862, angiostatin, vitaxin, droloxifene, idoxyfene, spironolactone, finasteride, cimitidine, trastuzumab, denileukin diftitox, gefitinib, bortezimib, paclitaxel, cremophor-free paclitaxel, docetaxel, epithilone B, BMS-247550, BMS-310705, droloxifene, 4-hydroxytamoxifen, pipendoxifene, ERA-923, arzoxifene, fulvestrant, acolbifene, lasofoxifene, idoxifene, TSE-424, HMR-3339, ZK186619, topotecan, PTK787/ZK 222584, VX-745, PD 184352, rapamycin, 40-O-(2-hydroxyethyl)-rapamycin, temsirolimus, AP-23573, RAD001, ABT-578, BC-210, LY294002, LY292223, LY292696, LY293684, LY293646, wortmannin, ZM336372, L-779,450, PEG-filgrastim, darbepoetin, 5-fluorouracil, erythropoietin, granulocyte colony-stimulating factor, zolendronate, prednisone, cetuximab, granulocyte macrophage colony-stimulating factor, histrelin, pegylated interferon alfa-2a, interferon alfa-2a, pegylated interferon alfa-2b, interferon alfa-2b, azacitidine, PEG-L-asparaginase, lenalidomide, gemtuzumab, hydrocortisone, interleukin-11, dexrazoxane, alemtuzumab, all-transretinoic acid, ketoconazole, interleukin-2, megestrol, immune globulin, nitrogen mustard, methylprednisolone, ibritgumomab tiuxetan, androgens, decitabine, hexamethylmelamine, bexarotene, tositumomab, arsenic trioxide, cortisone, editronate, mitotane, cyclosporine, liposomal daunorubicin, Edwina-asparaginase, strontium 89, casopitant, netupitant, an NK-1 receptor antagonists, palonosetron, aprepitant, diphenhydramine, hydroxyzine, metoclopramide, lorazepam, alprazolam, haloperidol, droperidol, dronabinol, dexamethasone, methylprednisolone, prochlorperazine, granisetron, ondansetron, dolasetron, tropisetron, pegfilgrastim, erythropoietin, epoetin alfa and darbepoetin alfa. The present invention also provides the antibody or fragment bound to IGF1R or a soluble fragment thereof, in a complex.

The present invention also provides an isolated antibody or antigen-binding fragment thereof that binds specifically to IGF1R comprising one or more members selected from the group consisting of: (a) 2C6 CDR-H1 defined by SEQ ID NO: 2, 2C6 CDR-H2 defined by SEQ ID NO: 3 and 2C6 CDR-H3 defined by SEQ ID NO: 4; (b) 9H2CDR-H1 defined by SEQ ID NO: 10, 9H2CDR-H2 defined by SEQ ID NO: 11 and 9H2CDR-H3 defined by SEQ ID NO: 12; (c) 2C6 CDR-L1 defined by SEQ ID NO: 6, 2C6 CDR-L2 defined by SEQ ID NO: 7 and 2C6 CDR-L3 defined by SEQ ID NO: 8; and (d) 9H2CDR-L1 defined by SEQ ID NO: 14, 9H2CDR-L2 defined by SEQ ID NO: 15 and 9H2CDR-L3 defined by SEQ ID NO: 16. In an embodiment of the invention, the antibody is monoclonal, polyclonal, recombinant, humanized, bispecific, anti-idiotypic, chimeric or labeled. In an embodiment of the invention, the fragment is a single-chain variable fragment, an Fv fragment, a Fab fragment, Fab′ fragment, a F(ab′)₂ fragment, a disulfide-linked Fv. In an embodiment of the invention, the antibody or fragment is linked to a constant region such as a K light chain, a γ1 heavy chain, a γ2 heavy chain, a γ3 heavy chain or a γ4 heavy chain. The present invention also provides a pharmaceutical composition comprising the antibody or fragment in association with a pharmaceutically acceptable carrier. The present invention also provides a composition comprising the antibody or fragment in association with a further chemotherapeutic agent such as pemetrexed, erlotinib, dasatanib, nilotinib, decatanib, panitumumab, amrubicin, oregovomab, Lep-etu, nolatrexed, azd2171, batabulin, ofatumumab, zanolimumab, edotecarin, tetrandrine, rubitecan, tesmilifene, oblimersen, ticilimumab, ipilimumab, gossypol, Bio 111, 131-I-TM-601, ALT-110, BIO 140, CC 8490, cilengitide, gimatecan, IL13-PE38QQR, INO 1001, IPdR, KRX-0402, lucanthone, LY 317615, neuradiab, vitespan, Rta 744, Sdx 102, talampanel, atrasentan, Xr 311, romidepsin, ADS-100380,

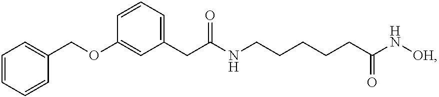

### CG-781, CG-1521,

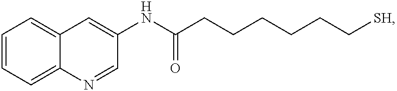

SB-556629, chlamydocin, JNJ-16241199,

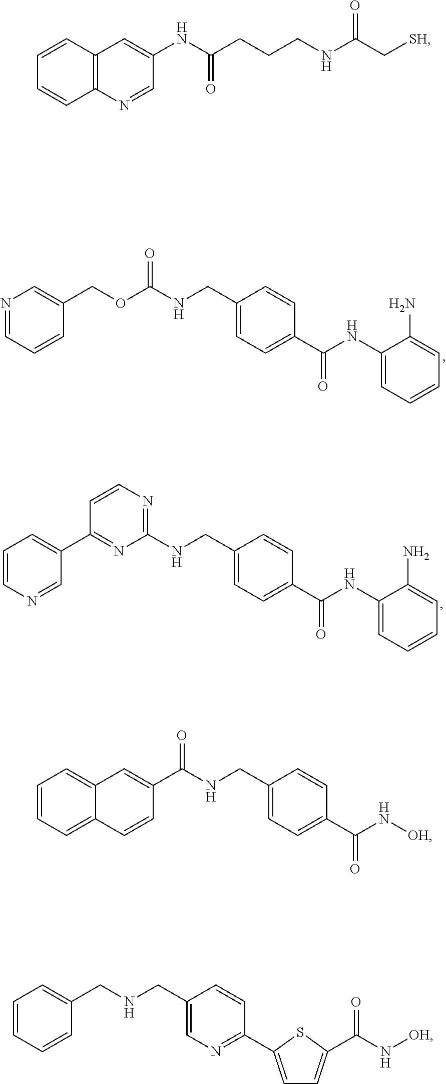

vorinostat, etoposide, gemcitabine, doxorubicin, liposomal doxorubicin, 5′-deoxy-5-fluorouridine, vincristine, temozolomide, ZK-304709, seliciclib; PD0325901, AZD-6244, capecitabine, L-Glutamic acid, N-[4-[2-(2-amino-4,7-dihydro-4-oxo-1H-pyrrolo[2,3-d]pyrimidin-5-yl)ethyl]benzoyl]-, disodium salt, heptahydrate, camptothecin, irinotecan; a combination of irinotecan, 5-fluorouracil and leucovorin; PEG-labeled irinotecan, FOLFOX regimen, tamoxifen, toremifene citrate, anastrazole, exemestane, letrozole, DES(diethylstilbestrol), estradiol, estrogen, conjugated estrogen, bevacizumab, IMC-1C11, CHIR-258,

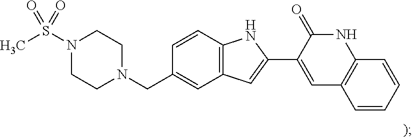

3-[5-(methylsulfonylpiperadinemethyl)-indolyl]-quinolone, vatalanib, AG-013736, AVE-0005, the acetate salt of [D-Ser(Bu t)6, Azgly 10] (pyro-Glu-His-Trp-Ser-Tyr-D-Ser(Bu t)-Leu-Arg-Pro-Azgly-NH2 acetate [C₅₉H84N₁₈O₁₄.(O₂H4O₂)ₓ where x=1 to 2.4], goserelin acetate, leuprolide acetate, triptorelin pamoate, sunitinib, sunitinib malate, medroxyprogesterone acetate, hydroxyprogesterone caproate, megestrol acetate, raloxifene, bicalutamide, flutamide, nilutamide, megestrol acetate, CP-724714; TAK-165, HKI-272, erlotinib, lapatanib, canertinib, ABX-EGF antibody, erbitux, EKB-569, PKI-166, GW-572016, lonafarnib,

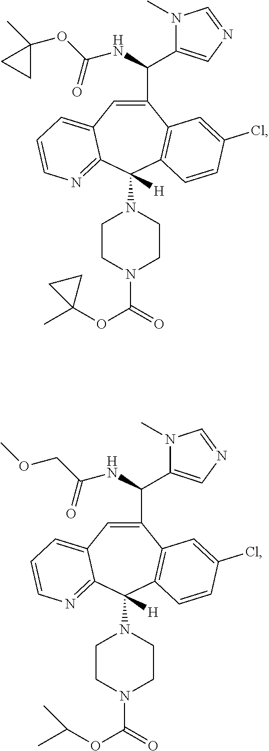

BMS-214662, tipifarnib; amifostine, NVP-LAQ824, suberoyl analide hydroxamic acid, valproic acid, trichostatin A, FK-228, SU11248, sorafenib, KRN951, aminoglutethimide, amsacrine, anagrelide, L-asparaginase, Bacillus Calmette-Guerin (BCG) vaccine, bleomycin, buserelin, busulfan, carboplatin, carmustine, chlorambucil, cisplatin, cladribine, clodronate, cyclophosphamide, cyproterone, cytarabine, dacarbazine, dactinomycin, daunorubicin, diethylstilbestrol, epirubicin, fludarabine, fludrocortisone, fluoxymesterone, flutamide, hydroxyurea, idarubicin, ifosfamide, imatinib, leucovorin, leuprolide, levamisole, lomustine, mechlorethamine, melphalan, 6-mercaptopurine, mesna, methotrexate, mitomycin, mitotane, mitoxantrone, nilutamide, octreotide, oxaliplatin, pamidronate, pentostatin, plicamycin, porfimer, procarbazine, raltitrexed, rituximab, streptozocin, teniposide, testosterone, thalidomide, thioguanine, thiotepa, tretinoin, vindesine, 13-cis-retinoic acid, phenylalanine mustard, uracil mustard, estramustine, altretamine, floxuridine, 5-deooxyuridine, cytosine arabinoside, 6-mercaptopurine, deoxycoformycin, calcitriol, valrubicin, mithramycin, vinblastine, vinorelbine, topotecan, razoxin, marimastat, COL-3, neovastat, BMS-275291, squalamine, endostatin, SU5416, SU6668, EMD121974, interleukin-12, IM862, angiostatin, vitaxin, droloxifene, idoxyfene, spironolactone, finasteride, cimitidine, trastuzumab, denileukin diftitox, gefitinib, bortezimib, paclitaxel, cremophor-free paclitaxel, docetaxel, epithilone B, BMS-247550, BMS-310705, droloxifene, 4-hydroxytamoxifen, pipendoxifene, ERA-923, arzoxifene, fulvestrant, acolbifene, lasofoxifene, idoxifene, TSE-424, HMR-3339, ZK186619, topotecan, PTK787/ZK 222584, VX-745, PD 184352, rapamycin, 40-O-(2-hydroxyethyl)-rapamycin, temsirolimus, AP-23573, RAD001, ABT-578, BC-210, LY294002, LY292223, LY292696, LY293684, LY293646, wortmannin, ZM336372, L-779,450, PEG-filgrastim, darbepoetin, 5-fluorouracil, erythropoietin, granulocyte colony-stimulating factor, zolendronate, prednisone, cetuximab, granulocyte macrophage colony-stimulating factor, histrelin, pegylated interferon alfa-2a, interferon alfa-2a, pegylated interferon alfa-2b, interferon alfa-2b, azacitidine, PEG-L-asparaginase, lenalidomide, gemtuzumab, hydrocortisone, interleukin-11, dexrazoxane, alemtuzumab, all-transretinoic acid, ketoconazole, interleukin-2, megestrol, immune globulin, nitrogen mustard, methylprednisolone, ibritgumomab tiuxetan, androgens, decitabine, hexamethylmelamine, bexarotene, tositumomab, arsenic trioxide, cortisone, editronate, mitotane, cyclosporine, liposomal daunorubicin, Edwina-asparaginase, strontium 89, casopitant, netupitant, an NK-1 receptor antagonists, palonosetron, aprepitant, diphenhydramine, hydroxyzine, metoclopramide, lorazepam, alprazolam, haloperidol, droperidol, dronabinol, dexamethasone, methylprednisolone, prochlorperazine, granisetron, ondansetron, dolasetron, tropisetron, pegfilgrastim, erythropoietin, epoetin alfa and darbepoetin alfa. The present invention also provides the antibody or fragment bound to IGF1R or a soluble fragment thereof, in a complex.

The present invention provides an isolated antibody or antigen-binding fragment thereof comprising one or more members selected from the group consisting of: (a) a mature heavy immunoglobulin chain variable region of SEQ ID NO: 1; (b) a mature light immunoglobulin chain variable region of SEQ ID NO: 5; (c) a mature heavy immunoglobulin chain variable region of SEQ ID NO: 9; and (d) a mature light immunoglobulin chain variable region of SEQ ID NO: 13. In an embodiment of the invention, the antibody is monoclonal, polyclonal, recombinant, humanized, bispecific, anti-idiotypic, chimeric or labeled. In an embodiment of the invention, the fragment is a single-chain variable fragment, an Fv fragment, a Fab fragment, Fab′ fragment, a F(ab′)₂ fragment, a disulfide-linked Fv. In an embodiment of the invention, the antibody or fragment is linked to a constant region such as a K light chain, a γ1 heavy chain, a γ2 heavy chain, a γ3 heavy chain or a γ4 heavy chain. The present invention provides a pharmaceutical composition comprising the antibody or fragment in association with a pharmaceutically acceptable carrier. The present invention also provides a composition comprising the antibody or fragment in association with a further chemotherapeutic agent such as pemetrexed, erlotinib, dasatanib, nilotinib, decatanib, panitumumab, amrubicin, oregovomab, Lep-etu, nolatrexed, azd2171, batabulin, ofatumumab, zanolimumab, edotecarin, tetrandrine, rubitecan, tesmilifene, oblimersen, ticilimumab, ipilimumab, gossypol, Bio 111, 131-I-TM-601, ALT-110, BIO 140, CC 8490, cilengitide, gimatecan, IL13-PE38QQR, INO 1001, IPdR, KRX-0402, lucanthone, LY 317615, neuradiab, vitespan, Rta 744, Sdx 102, talampanel, atrasentan, Xr 311, romidepsin, ADS-100380,

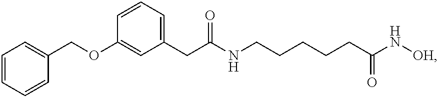

### CG-781, CG-1521,

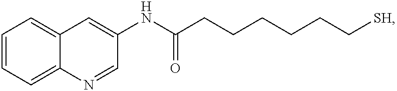

### SB-556629,

chlamydocin, JNJ-16241199,

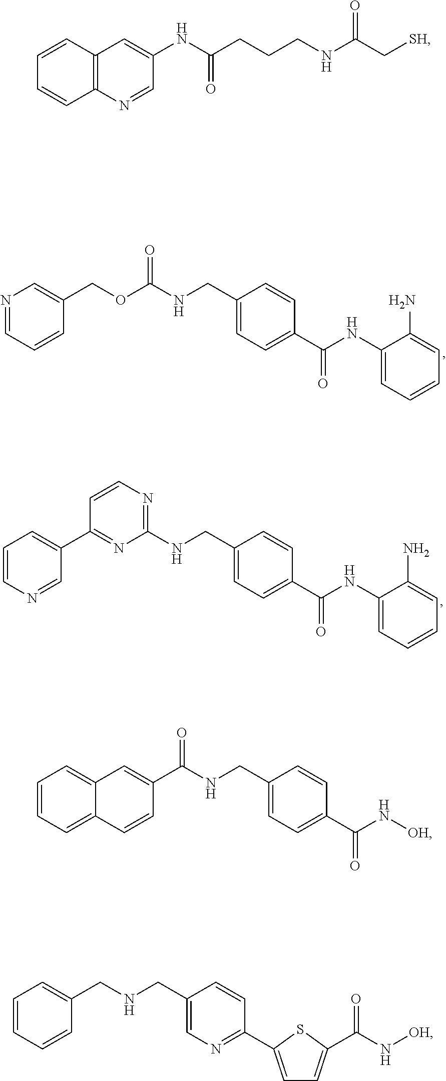

vorinostat, etoposide, gemcitabine, doxorubicin, liposomal doxorubicin, 5′-deoxy-5-fluorouridine, vincristine, temozolomide, ZK-304709, seliciclib; PD0325901, AZD-6244, capecitabine, L-Glutamic acid, N-[4-[2-(2-amino-4,7-dihydro-4-oxo-1H-pyrrolo[2,3-d]pyrimidin-5-yl)ethyl]benzoyl]-, disodium salt, heptahydrate, camptothecin, irinotecan; a combination of irinotecan, 5-fluorouracil and leucovorin; PEG-labeled irinotecan, FOLFOX regimen, tamoxifen, toremifene citrate, anastrazole, exemestane, letrozole, DES(diethylstilbestrol), estradiol, estrogen, conjugated estrogen, bevacizumab, IMC-1C11, CHIR-258,

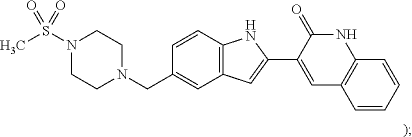

3-[5-(methylsulfonylpiperadinemethyl)-indolyl]-quinolone, vatalanib, AG-013736, AVE-0005, the acetate salt of [D-Ser(Bu t)6, Azgly 10] (pyro-Glu-His-Trp-Ser-Tyr-D-Ser(Bu t)-Leu-Arg-Pro-Azgly-NH2 acetate [C₅₉H84N₁₈O₁₄.(C₂H4O₂)ₓ where x=1 to 2.4], goserelin acetate, leuprolide acetate, triptorelin pamoate, sunitinib, sunitinib malate, medroxyprogesterone acetate, hydroxyprogesterone caproate, megestrol acetate, raloxifene, bicalutamide, flutamide, nilutamide, megestrol acetate, CP-724714; TAK-165, HKI-272, erlotinib, lapatanib, canertinib, ABX-EGF antibody, erbitux, EKB-569, PKI-166, GW-572016,

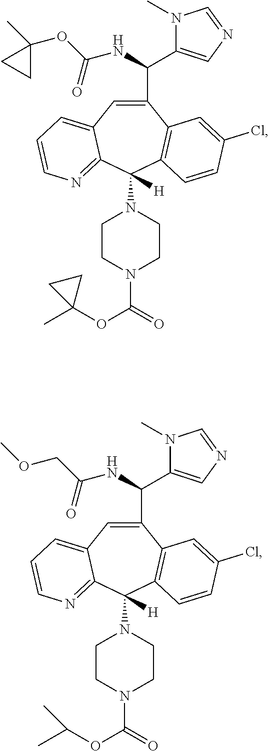

lonafarnib, BMS-214662, tipifarnib; amifostine, NVP-LAQ824, suberoyl analide hydroxamic acid, valproic acid, trichostatin A, FK-228, SU11248, sorafenib, KRN951, aminoglutethimide, amsacrine, anagrelide, L-asparaginase, Bacillus Calmette-Guerin (BCG) vaccine, bleomycin, buserelin, busulfan, carboplatin, carmustine, chlorambucil, cisplatin, cladribine, clodronate, cyclophosphamide, cyproterone, cytarabine, dacarbazine, dactinomycin, daunorubicin, diethylstilbestrol, epirubicin, fludarabine, fludrocortisone, fluoxymesterone, flutamide, hydroxyurea, idarubicin, ifosfamide, imatinib, leucovorin, leuprolide, levamisole, lomustine, mechlorethamine, melphalan, 6-mercaptopurine, mesna, methotrexate, mitomycin, mitotane, mitoxantrone, nilutamide, octreotide, oxaliplatin, pamidronate, pentostatin, plicamycin, porfimer, procarbazine, raltitrexed, rituximab, streptozocin, teniposide, testosterone, thalidomide, thioguanine, thiotepa, tretinoin, vindesine, 13-cis-retinoic acid, phenylalanine mustard, uracil mustard, estramustine, altretamine, floxuridine, 5-deooxyuridine, cytosine arabinoside, 6-mercaptopurine, deoxycoformycin, calcitriol, valrubicin, mithramycin, vinblastine, vinorelbine, topotecan, razoxin, marimastat, COL-3, neovastat, BMS-275291, squalamine, endostatin, SU5416, SU6668, EMD121974, interleukin-12, IM862, angiostatin, vitaxin, droloxifene, idoxyfene, spironolactone, finasteride, cimitidine, trastuzumab, denileukin diftitox, gefitinib, bortezimib, paclitaxel, cremophor-free paclitaxel, docetaxel, epithilone B, BMS-247550, BMS-310705, droloxifene, 4-hydroxytamoxifen, pipendoxifene, ERA-923, arzoxifene, fulvestrant, acolbifene, lasofoxifene, idoxifene, TSE-424, HMR-3339, ZK186619, topotecan, PTK787/ZK 222584, VX-745, PD 184352, rapamycin, 40-O-(2-hydroxyethyl)-rapamycin, temsirolimus, AP-23573, RAD001, ABT-578, BC-210, LY294002, LY292223, LY292696, LY293684, LY293646, wortmannin, ZM336372, L-779,450, PEG-filgrastim, darbepoetin, 5-fluorouracil, erythropoietin, granulocyte colony-stimulating factor, zolendronate, prednisone, cetuximab, granulocyte macrophage colony-stimulating factor, histrelin, pegylated interferon alfa-2a, interferon alfa-2a, pegylated interferon alfa-2b, interferon alfa-2b, azacitidine, PEG-L-asparaginase, lenalidomide, gemtuzumab, hydrocortisone, interleukin-11, dexrazoxane, alemtuzumab, all-transretinoic acid, ketoconazole, interleukin-2, megestrol, immune globulin, nitrogen mustard, methylprednisolone, ibritgumomab tiuxetan, androgens, decitabine, hexamethylmelamine, bexarotene, tositumomab, arsenic trioxide, cortisone, editronate, mitotane, cyclosporine, liposomal daunorubicin, Edwina-asparaginase, strontium 89, casopitant, netupitant, an NK-1 receptor antagonists, palonosetron, aprepitant, diphenhydramine, hydroxyzine, metoclopramide, lorazepam, alprazolam, haloperidol, droperidol, dronabinol, dexamethasone, methylprednisolone, prochlorperazine, granisetron, ondansetron, dolasetron, tropisetron, pegfilgrastim, erythropoietin, epoetin alfa and darbepoetin alfa. The present invention further provides the antibody or fragment bound to IGF1R or a soluble fragment thereof, in a complex.

The present invention additionally provides an isolated polypeptide comprising an amino acid sequence selected from the group consisting of SEQ ID NOs: 1-16, amino acids 20-144 of SEQ ID NO: 1, amino acids 23-129 of SEQ ID NO: 5, amino acids 20-146 of SEQ ID NO: 9 and amino acids 21-130 of SEQ ID NO: 13; along with any isolated polynucleotide encoding such polypeptide; any isolated vector comprising the polynucleotide; any isolated host cell (e.g., bacterial (e.g., E. coli) or mammalian) comprising the vector.

The present invention further provides a method for treating or preventing a medical condition, in a subject, mediated by elevated expression or activity of insulin-like growth factor I receptor or elevated expression of IGF-1 or elevated expression of IGF-II comprising administering (e.g., by a parenteral route) a therapeutically effective amount of any antibody or fragment described herein (optionally in association with an additional chemotherapeutic agent (e.g., as discussed herein) or anti-cancer therapeutic procedure (e.g., surgical tumorectomy or anti-cancer radiation therapy)) to said subject. In an embodiment of the invention, the medical condition is a member selected from the group consisting of osteosarcoma, rhabdomyosarcoma, neuroblastoma, any pediatric cancer, kidney cancer, leukemia, renal transitional cell cancer, Werner-Morrison syndrome, acromegaly, bladder cancer, Wilm's cancer, ovarian cancer, pancreatic cancer, benign prostatic hyperplasia, breast cancer, prostate cancer, bone cancer, lung cancer, gastric cancer, colorectal cancer, cervical cancer, synovial sarcoma, diarrhea associated with metastatic carcinoid, vasoactive intestinal peptide secreting tumors, gigantism, psoriasis, atherosclerosis, smooth muscle restenosis of blood vessels and inappropriate microvascular proliferation, head and neck cancer, squamous cell carcinoma, multiple myeloma, solitary plasmacytoma, renal cell cancer, retinoblastoma, germ cell tumors, hepatoblastoma, hepatocellular carcinoma, melanoma, rhabdoid tumor of the kidney, Ewing Sarcoma, chondrosarcoma, haemotological malignancy, chronic lymphoblastic leukemia, chronic myelomonocytic leukemia, acute lymphoblastic leukemia, acute lymphocytic leukemia, acute myelogenous leukemia, acute myeloblastic leukemia, chronic myeloblastic leukemia, Hodgekin's disease, non-Hodgekin's lymphoma, chronic lymphocytic leukemia, chronic myelogenous leukemia, myelodysplastic syndrome, hairy cell leukemia, mast cell leukemia, mast cell neoplasm, follicular lymphoma, diffuse large cell lymphoma, mantle cell lymphoma, Burkitt Lymphoma, mycosis fungoides, seary syndrome, cutaneous T-cell lymphoma, chronic myeloproliferative disorders, a cental nervous system tumor, brain cancer, glioblastoma, non-glioblastoma brain cancer, meningioma, pituitary adenoma, vestibular schwannoma, a primitive neuroectodermal tumor, medulloblastoma, astrocytoma, anaplastic astrocytoma, oligodendroglioma, ependymoma and choroid plexus papilloma, a myeloproliferative disorder, polycythemia vera, thrombocythemia, idiopathic myelfibrosis, soft tissue sarcoma, thyroid cancer, endometrial cancer, carcinoid cancer, germ cell tumors, liver cancer, gigantism, psoriasis, atherosclerosis, smooth muscle restenosis of blood vessels, inappropriate microvascular proliferation, acromegaly, gigantism, psoriasis, atherosclerosis, smooth muscle restenosis of blood vessels or inappropriate microvascular proliferation, Grave's disease, multiple sclerosis, systemic lupus erythematosus, Hashimoto's Thyroiditis, Myasthenia Gravis, auto-immune thyroiditis and Bechet's disease.

The present invention also provides a method of detecting the presence or location of an IGF1R-expressing tumor in a subject, comprising (i) administering the antibody or fragment of claim 1 to the subject; and (ii) detecting binding of the antibody or fragment, wherein said binding indicates the presence or location of said tumor.

The present invention provides an isolated monoclonal antibody comprising a heavy chain immunoglobulin variable region comprising amino acids 20-144 of SEQ ID NO: 1 linked to a gamma 1 immunoglobulin constant region and a light chain immunoglobulin variable region comprising amino acids 23-129 of SEQ ID NO: 5 linked to a kappa immunoglobulin constant region; the present invention also provides an isolated monoclonal antibody comprising a heavy chain immunoglobulin variable region comprising amino acids 20-146 of SEQ ID NO: 9 linked to a gamma 1 immunoglobulin constant region and a light chain immunoglobulin variable region comprising amino acids 21-130 of SEQ ID NO: 13 linked to a kappa immunoglobulin constant region.

The present invention provides an isolated hybridoma cell having American Type Culture Collection deposit number PTA-8428 or PTA-8429 along with any antibody produced by such a hybridoma. The present invention further provides a method for producing an isolated antibody comprising culturing the hybridoma cell in a culture medium under conditions suitable for expression of said antibody by said hybridoma and, optionally, purifying the antibody from the culture medium. Any antibody produced by such a hybridoma also is within the scope of the present invention.

### DETAILED DESCRIPTION OF THE INVENTION

The present invention includes fully human, monoclonal antibodies and antigen-binding fragment thereof which specifically recognize and bind to Insulin-like Growth Factor Receptor-I (IGF1R), for example, a soluble fragment thereof comprising amino acids 1-932 thereof or amino acids 30-902 of SEQ ID NO: 17. In an embodiment of the invention, the antibody is 2C6 or 9H2.

Antibodies include whole antibodies (e.g., IgG, preferably, IgG1 or IgG4). Antigen-binding fragments of antibodies include Fab antibody fragments, F(ab)₂ antibody fragments, Fv antibody fragments, single chain Fv antibody fragments and dsFv antibody fragments which bind to an antigen such as IGF1R or a fragment thereof.

The terms “IGF1R”, “IGFR1” “Insulin-like Growth Factor Receptor-I” and “Insulin-like Growth Factor Receptor, type I” are well known in the art. Although IGF1R may be from any organism, it is, in an embodiment of the invention, from an animal, such as a mammal (e.g., mouse, rat, rabbit, sheep or dog), e.g., a human. The nucleotide and amino acid sequence of a typical human IGF1R precursor has the Genbank Accession No. X04434 or NM_{—}000875 (SEQ ID NO: 17). Cleavage of the precursor (e.g., between amino acids 710 and 711) produces an α-subunit and a β-subunit which associate to form a mature receptor. In particular embodiments of the invention, amino acids 30-902, from the full length IGF1R polypeptide are used as an antigen for generation of anti-IGF1R antibodies. The term “sIGF1R” or “soluble IGF1R” includes any soluble fragment of IGF1R (e.g., human IGF1R), e.g., a fragment from which the receptor trans-membrane region has been deleted, e.g., amino acids 30-902 of SEQ ID NO: 17, e.g., amino acids 1-932 of IGF1R.

The terms “IGF-I” “Insulin-like Growth Factor-I” and “Insulin-like Growth Factor, type I” are also well known in the art. The terms “IGF-II” “Insulin-like Growth Factor-II” and “Insulin-like Growth Factor, type II” are also well known in the art. Although IGF-I or IGF-II may be from any organism, they are, in an embodiment of the invention, from an animal, such as a mammal (e.g., mouse, rat, rabbit, sheep or dog) such as a human. The nucleic acid and amino acid sequence of typical, human IGF-I and IGF-II have the Genbank Accession No. XM_{—}052648 and NM_{—}000612, respectively.

The term “isolated” or the like refers to the purification status of a substance, such as an antibody or antigen-binding fragment thereof and in such a context means the substance has been purified from other biological substances (e.g., components of a cell in which the substance is produced) to any degree whatsoever. In an embodiment of the invention, the substance is substantially free of other biological molecules such as nucleic acids, proteins, lipids, carbohydrates, or other material such as cellular debris and growth media. In an embodiment of the invention, an isolated substance has been removed from other contaminating substances essentially completely.

The amino acid sequence of the variable region of human, monoclonal anti-IGF1R antibodies of the invention are summarized in Table 1. The present invention includes any isolated polypeptide set forth in Table 1 below (including mature fragments thereof which lack a signal peptide) along with any isolated polynucleotide encoding such a polypeptide or fragment and any vector (e.g., a recombinant vector such as a plasmid) comprising such a polynucleotide and any isolated host cell comprising such a vector.

TABLE 1
Summary of amino acid and nucleotide sequences.

| Sequence Description | SEQ ID NO: |
|---|---|
| Antibody 2C6 heavy chain variable region | 1 |
| 2C6 CDR-H1 | 2 |
| 2C6 CDR-H2 | 3 |
| 2C6 CDR-H3 | 4 |
| Antibody 2C6 light chain variable region | 5 |
| 2C6 CDR-L1 | 6 |
| 2C6 CDR-L2 | 7 |
| 2C6 CDR-L3 | 8 |
| Antibody 9H2 heavy chain variable region | 9 |
| 9H2 CDR-H1 | 10 |
| 9H2 CDR-H2 | 11 |
| 9H2 CDR-H3 | 12 |
| Antibody 9H2 light chain variable region | 13 |
| 9H2 CDR-L1 | 14 |
| 9H2 CDR-L2 | 15 |
| 9H2 CDR-L3 | 16 |
| Human IGF1R | 17 |
| 15H12/19D12 anti-IGF1R antibody chains | 18-23 |

### Antibodies

Immunoglobulin variable regions of the present invention are set forth below. The underscored text denotes signal sequence and bolded text denotes complementarity determining regions (CDRs). A mature chain lacks the signal sequence.

2C6 HEAVY CHAIN
(SEQ ID NO: 1)
MELGLSWIFLLAILKGVQC
EVQLVESGGGLVQPGRSLRLSCAASGFTFDDYAMHWVRQAPGKGLEWV
SGISWNSGSKGYVDSVKGRFTISRDNAKNSLYLQMNSLRAEDTALYYC
AKDIRIGVAASYYFGEDVWGHGTTVTVSS
2C6 CDR-H1:
(SEQ ID NO: 2)
GFTFDDYAMH
2C6 CDR-H2:
(SEQ ID NO: 3)
GISWNSGSKGYVDSVKG
2C6 CDR-H3:
(SEQ ID NO: 4)
DIRIGVAASYYFGMDV
2C6 LIGHT CHAIN
(SEQ ID NO: 5)
MDMRVPAQLLGLLLLWLPGARC
AIQLTQSPSSLSASVGDRVTITCRASQGISSVLAWYQQKPGKAPKLLI
YDASSLESGVPSRFSGSGSGTDFTLTISSLQPEDFATYYCQQFNSYPY
TFGQGTKLEIK
2C6 CDR-L1:
(SEQ ID NO: 6)
RASQGISSVLA
2C6 CDR-L2:
(SEQ ID NO: 7)
DASSLES
2C6 CDR-L3:
(SEQ ID NO: 8)
QQFNSYPYT
9H2 HEAVY CHAIN
(SEQ ID NO: 9)
MDWTWRILFLVAAATGAHS
QVQLVQSGAEVKKPGASVKVSCKASGYTFTSYVMHWVRQAPGQRLEWM
GWINAGNGNTRYSQKFQGRVTITRDTSASTVYMELSSLRSEDTAVYYC
ARGGMPVAGPGYFYYYGMDVWGQGTTVTVSS
9H2 CDR-H1:
(SEQ ID NO: 10)
GYTFTSYVMH
9H2 CDR-H2:
(SEQ ID NO: 11)
WINAGNGNTKYSQKFQG
9H2 CDR-H3:
(SEQ ID NO: 12)
GGMPVAGPGYFYYYGMDV
9H2 LIGHT CHAIN
(SEQ ID NO: 13)
METPAQLLFLLLLWLPDTTG
EIVLTQSPGTLSLSPGERATLSCRASQSVSRSYLAWYQQKPGQAPRLL
IYGASSRATGIPDRFSGSGSGTDFTLTISRLEPEDFAVYCCQQYGSSP
WTFGQGTKVEIKRT
9H2 CDR-L1:
(SEQ ID NO: 14)
RASQSVSRSYLA
9H2 CDR-L2:
(SEQ ID NO: 15)
GASSRAT
9H2 CDR-L3:
(SEQ ID NO: 16)
QQYGSSPWT

The present invention comprises any isolated polypeptide comprising or consisting of an amino acid selected from the group consisting of SEQ ID Nos: 1-16 as well as any isolated polynucleotide encoding such a polypeptide; any vector comprising such a polynucleotide and any host cell including such a vector.

The present invention comprises any isolated antibody or antigen-binding fragment thereof comprising one or more of the foregoing mature heavy and/or light chain variable regions and/or CDRs. In an embodiment of the invention, the antibody comprises a mature heavy chain of the polypeptide comprising the amino acid sequence of SEQ ID NO: 1 and a mature light chain of the polypeptide comprising the amino acid sequence of SEQ ID NO:5; or the antibody comprises a mature heavy chain of the polypeptide comprising the amino acid sequence of SEQ ID NO: 9 and a mature light chain of the polypeptide comprising the amino acid sequence of SEQ ID NO: 13; e.g., linked to an immunoglobulin constant region.

The present invention includes isolated polypeptides (e.g., antibodies and antigen-binding fragments thereof) comprising one or more (e.g., 3) CDRs taken from the 2C6 and 9H2 light or heavy chain immunoglobulin as defined by the convention set forth in Kabat, “Sequences of Proteins of Immunological Interest” (National Institutes of Health, Bethesda, Md., 1987 and 1991) or in Chothia et al., J. Mol. Biol. 196:901 (1987); Nature 342:878 (1989); and J. Mol. Biol. 186:651 (1989) (Kabat or Chothia) or Al-Lazikani et al., J. Mol. Biol. 273: 927-948 (1997).

In an embodiment of the invention, an antibody or antigen-binding fragment thereof comprises CDRs taken from one or both of antibodies 2C6 and 9H2 which are arranged in any order whatsoever and on any chain whatsoever (heavy or light) without respect to the context of the CDR as it exists in the original 2C6 or 9H2 antibody.

For example, in an embodiment of the invention, an antibody or antigen-binding fragment thereof comprises the mature 2C6 light chain CDRs in the context of another immunoglobulin framework, e.g.,

(SEQ ID NO: 24)
E I V L T Q S P G T L S V S P G E R A T L
S C RASQGISSVLA W Y Q Q K P G Q A P R L L I K
DASSLES G I P D R F S G S G S G T D F T L T
I S R L E P E D F A V Y Y C QQFNSYPYT F G Q G
T K V E I K R T

or 9H2 mature light chain CDRs in the context of another immunoglobulin framework, e.g.,

(SEQ ID NO: 25)
E I V L T Q S P G T L S V S P G E R A T L
S C RASQSVSRSYLA W Y Q Q K P G Q A P R L L I K
GASSRAT G I P D R F S G S G S G T D F T L T
I S R L E P E D F A V Y Y C QQYGSSPWT F G Q G
T K V E I K R T;

wherein the CDRs are in bold faced font.

For example, in an embodiment of the invention, an antibody or antigen-binding fragment thereof comprises the mature 2C6 heavy chain CDRs in the context of another immunoglobulin framework, e.g., EVQLVQSGGGLVKPGGSLRLSCAASGFTFDDYAMIENVRQAPGKGLEWIS GISWNSGSRGYVDSVKGRFTISRDNAKNSLYLQMNSLRAEDTAVYYCARDIRIGVAASYYFGMDVWGQGTTVT VSS; (SEQ ID NO: 26); wherein the CDRs are in bold faced font.

For example, in an embodiment of the invention, an antibody or antigen-binding fragment thereof comprises the mature 9H2 heavy chain CDRs in the context of another immunoglobulin framework, e.g., VQLVQSGGGLVKPGGSLRLSCAASGYTFTSYVMHWVRQAPGKGLEWIS WINAGNGNTKYSQKFQGRFTISRDNAKNSLYLQMNSLRAEDTAVYYCARGGMPVAGPGYFYYYGMDV WGQGTTVTVSS (SEQ ID NO: 27); wherein the CDRs are in bold faced font.

The present invention also includes versions of the heavy and light chain immunoglobulins of the present invention (e.g., including mature fragments thereof) which comprise a one conservative substitution of a framework of said immunoglobulin; wherein binding to IGF1R is preserved to any degree (e.g., wherein affinity for IGF1R is essentially the same as that of the native immunoglobulin). Versions comprising more than one substitution also are part of the present invention. For example, an antibody or antigen-binding fragment thereof comprising a variant of the 2C6 heavy chain immunoglobulin comprising any of the following mutations is within the scope of the present invention: L30I, L30V, K62R, E65D. Similarly, conservatively substituted versions of any of the other immunoglobulins of the present invention (e.g., 2C6 light chain, 9H2 heavy chain or 9H2 light chain) also form part of the present invention.

The present invention includes anti-IGF1R antibodies and antigen-binding fragments thereof. Thus, the invention includes monoclonal antibodies, camelized single domain antibodies, polyclonal antibodies, bispecific antibodies, chimeric antibodies, recombinant antibodies, anti-idiotypic antibodies, humanized antibodies, bispecific antibodies, diabodies, single chain antibodies, disulfide Fvs (dsfv), Fvs, Fabs, Fab's, F(ab′)₂s and domain antibodies. Thus, the term antibody covers, but is not limited to, monoclonal antibodies, polyclonal antibodies, multispecific antibodies (e.g., bispecific antibodies). The terms antigen-binding fragment of an antibody (of the “parental antibody”) encompass a fragment or a derivative of an antibody, typically including at least a portion of the antigen-binding or variable regions (e.g., one or more CDRs) of the parental antibody, that retains at least some of the binding specificity of the parental antibody. Examples of antibody antigen-binding fragments include, but are not limited to, Fab, Fab′, F(ab′)₂, and Fv fragments; diabodies; single-chain antibody molecules, e.g., sc-Fv; and multispecific antibodies formed from antibody fragments. Typically, a binding fragment or derivative retains at least 10% of its IGF1R binding activity when that activity is expressed on a molar basis. In an embodiment of the invention, a binding fragment or derivative retains at least 20%, 50%, 70%, 80%, 90%, 95% or 100% or more of the IGF1R binding affinity as the parental antibody. It is also intended that an IGF1R binding fragment can include conservative amino acid substitutions (referred to as “conservative variants” of the antibody) that do not substantially alter its biologic activity. The term “binding compound” refers to both antibodies and binding fragments thereof.

A “Fab fragment” is comprised of one light chain and the C_{H}1 and variable regions of one heavy chain. The heavy chain of a Fab molecule cannot form a disulfide bond with another heavy chain molecule.

An “Fc” region contains two heavy chain fragments comprising the C_{H}1 and C_{H}2 domains of an antibody. The two heavy chain fragments are held together by two or more disulfide bonds and by hydrophobic interactions of the C_{H}3 domains.

A “Fab′ fragment” contains one light chain and a portion of one heavy chain that contains the V_{H} domain and the C_{H}1 domain and also the region between the C_{H}1 and C_{H}2 domains, such that an interchain disulfide bond can be formed between the two heavy chains of two Fab′ fragments to form a F(ab′)₂ molecule.

A “F(ab′)₂ fragment” contains two light chains and two heavy chains containing a portion of the constant region between the C_{H}1 and C_{H}2 domains, such that an interchain disulfide bond is formed between the two heavy chains. A F(ab′)₂ fragment thus is composed of two Fab′ fragments that are held together by a disulfide bond between the two heavy chains.

“Disulfide stabilized Fv fragments” and “dsFv” include molecules having a variable heavy chain (V_{H}) and/or a variable light chain (V_{L}) which are linked by a disulfide bridge.

The “Fv region” comprises the variable regions from both the heavy and light chains, but lacks the constant regions.

The term “single-chain Fv” or “scFv” antibody refers to antibody fragments comprising the V_{H} and V_{L} domains of an antibody, wherein these domains are present in a single polypeptide chain. Generally, the Fv polypeptide further comprises a polypeptide linker between the V_{H} and V_{L} domains which enables the V_{H} and V_{L} chains to pair and form a binding site (e.g., 5-12 residues long). For a review of scFv, see Pluckthun (1994) THE PHARMACOLOGY OF MONOCLONAL ANTIBODIES, vol. 113, Rosenburg and Moore eds. Springer-Verlag, New York, pp. 269-315. See also, International Patent Application Publication No. WO 88/01649 and U.S. Pat. Nos. 4,946,778 and 5,260,203.

A “domain antibody” is an immunologically functional immunoglobulin fragment containing only the variable region of a heavy chain or the variable region of a light chain. In some instances, two or more V_{H} regions are covalently joined with a peptide linker to create a bivalent domain antibody. The two V_{H} regions of a bivalent domain antibody may target the same or different antigens.

A “bivalent antibody” comprises two antigen-binding sites. In some instances, the two binding sites have the same antigen specificities. However, bivalent antibodies may be bispecific. For example, the present invention comprises scfv dimers and dsfv dimers, each of which scfv and dsfv moieties may have a common or different antigen binding specificity.

In an embodiment of the invention, a (dsfv)₂ comprises three peptide chains: two V_{H} moieties linked by a peptide linker and bound by disulfide bridges to two V_{L} moieties. In an embodiment of the invention, a bispecific ds diabody comprises a VH1-VL₂ (tethered by a peptide linker) linked, by a disulfide bridge between the VH1 and VL₁, to a VL₁-VH2 moiety (also tethered by a peptide linker). In an embodiment of the invention, a bispecific dsfv-dsfv′ also comprises three peptide chains: a VH1-VH2 moiety wherein the heavy chains are linked by a peptide linker (e.g., a long flexible linker) and are bound to VL₁ and VL₂ moieties, respectively, by disulfide bridges; wherein each disulfide paired heavy and light chain has a different antigen specificity. In an embodiment of the invention, an scfv dimer (a bivalent diabody) comprises a V_{H}-V_{L} moiety wherein the heavy and light chains are bound to by a peptide linker and dimerized with another such moiety such that V_{H}s of one chain coordinate with the V_{L}s of another chain and form two identical binding sites. In an embodiment of the invention a bispecific diabody comprises VH1-VL₂ moiety (linked by a peptide linker) associated with a VL₁-VH2 (linked by a peptide linker), wherein the VH1 and VL₁ coordinate and the VH2 and VL₂ coordinate and each coordinated set has diverse antigen specificities.

The term “monoclonal antibody”, as used herein, refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic epitope. In contrast, conventional (polyclonal) antibody preparations typically include a multitude of antibodies directed against (or specific for) different epitopes. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made recombinantly or by the hybridoma method first described by Kohler et al. (1975) Nature 256: 495, or may be made by recombinant DNA methods (see, e.g., U.S. Pat. No. 4,816,567). The “monoclonal antibodies” may also be isolated from phage antibody libraries using the techniques described in Clackson et al. (1991) Nature 352: 624-628 and Marks et al. (1991) J. Mol. Biol. 222: 581-597, for example. See also Presta (2005) J. Allergy Clin. Immunol. 116:731.

Monoclonal antibodies include “chimeric” antibodies (immunoglobulins) in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (U.S. Pat. No. 4,816,567; and Morrison et al., (1984) Proc. Natl. Acad. Sci. USA 81: 6851-6855). For example, variable domains are obtained from an antibody from an experimental animal (the “parental antibody”), such as a human, and the constant domain sequences are obtained from canine antibodies, so that the resulting chimeric antibody will be less likely to elicit an adverse immune response in a canine subject than the parental human antibody.

A recombinant antibody or antigen-binding fragment thereof of the invention is, in an embodiment of the invention, an antibody which is produced recombinantly, e.g., expressed from a polynucleotide which has been introducted into an organism (e.g., a plasmid containing a polynucleotide encoding the antibody or fragment transformed into a bacterial cell (e.g., E. coli) or a mammalian cell (e.g., CHO cell)), followed by isolation of the antibody or fragment from the organism.

In an embodiment of the invention, anti-idiotypic antibodies or anti-idiotypes are antibodies directed against the antigen-combining region or variable region (called the idiotype) of another antibody molecule. As disclosed by Jerne (Jerne, N. K., (1974) Ann. Immunol. (Paris) 125c:373 and Jerne, N. K., et al., (1982) EMBO 1:234), immunization with an antibody molecule expressing a paratope (antigen-combining site) for a given antigen will produce a group of anti-antibodies, some of which share, with the antigen, a complementary structure to the paratope. Immunization with a subpopulation of the anti-idiotypic antibodies will, in turn, produce a subpopulation of antibodies or immune cell subsets that are reactive to the initial antigen.

The present invention also includes camelized single domain antibodies. See, e.g., Muyldermans et al. (2001) Trends Biochem. Sci. 26:230; Reichmann et al. (1999) J. Immunol. Methods 231:25; WO 94/04678; WO 94/25591; U.S. Pat. No. 6,005,079, which are hereby incorporated by reference in their entireties). Camelidae (camels, dromedaries and llamas) comprise IgG antibodies in which are devoid of light chains and therefore called ‘heavy-chain’ IgGs or HCAb (for heavy-chain antibody). HCAbs typically have a molecular weight of ˜95 kDa since they consist only of the heavy-chain variable domains. Although the HCAbs are devoid of light chains, they have an authentic antigen-binding repertoire (Hamers-Casterman et al., Nature (1993) 363:446-448; Nguyen et al., Adv. Immunol. (2001) 79:261-296; Nguyen et al., Immunogenetics. (2002) 54:39-47). In one embodiment, the present invention provides single domain antibodies comprising two V_{H} domains with modifications such that single domain antibodies are formed.

As used herein, the term “diabodies” refers to small antibody fragments with two antigen-binding sites, which fragments comprise a heavy chain variable domain (V_{H}) connected to a light chain variable domain (V_{L}) in the same polypeptide chain (V_{H}-V_{L} or V_{L}-V_{H}). By using a linker that is too short to allow pairing between the two domains on the same chain, the domains are forced to pair with the complementary domains of another chain and create two antigen-binding sites. Diabodies are described more fully in, e.g., EP 404,097; WO 93/11161; and Holliger et al. (1993) Proc. Natl. Acad. Sci. USA 90: 6444-6448. For a review of engineered antibody variants generally see Holliger and Hudson (2005) Nat. Biotechnol. 23:1126-1136.

As used herein, the term “humanized antibody” refers to forms of antibodies that contain sequences from both human and non-human (e.g., murine, rat) antibodies. In general, the humanized antibody will comprise substantially all of at least one, and typically two, variable domains, in which all or substantially all of the hypervariable loops correspond to those of a non-human immunoglobulin, and all or substantially all of the framework (FR) regions are those of a human immunoglobulin sequence. The humanized antibody may optionally comprise at least a portion of a human immunoglobulin constant region (Fc).

For example, the present invention comprises any humanized antibody comprising the CDRs of 9H2 or 2C6, e.g., wherein identical CDRs were originally isolated from a non-human species antibody and incorporated into a human antibody framework.

The antibodies of the present invention also include antibodies with modified (or blocked) Fc regions to provide altered effector functions. See, e.g., U.S. Pat. No. 5,624,821; WO2003/086310; WO2005/120571; WO2006/0057702. Such modifications can be used to enhance or suppress various reactions of the immune system, with possible beneficial effects in diagnosis and therapy. Alterations of the Fc region include amino acid changes (substitutions, deletions and insertions), glycosylation or deglycosylation, and adding multiple Fc. Changes to the Fc can also alter the half-life of antibodies in therapeutic antibodies, enabling less frequent dosing and thus increased convenience and decreased use of material. See Presta (2005) J. Allergy Clin. Immunol. 116:731 at 734-35.

The anti-IGF1R antibodies and antigen-binding fragments thereof of the invention are, in an embodiment of the invention, conjugated to a chemical moiety. The chemical moiety may be, inter alia, a polymer, a radionuclide or a cytotoxic factor. In an embodiment of the invention, the chemical moiety is a polymer which increases the half-life of the antibody or fragment in the body of a subject to whom it is administered. Polymers include, but are not limited to, polyethylene glycol (PEG) (e.g., PEG with a molecular weight of 2 kDa, 5 kDa, 10 kDa, 12 kDa, 20 kDa, 30 kDa or 40 kDa), dextran and monomethoxypolyethylene glycol (mPEG). Lee, et al., (1999) (Bioconj. Chem. 10:973-981) discloses PEG conjugated single-chain antibodies. Wen, et al., (2001) (Bioconj. Chem. 12:545-553) disclose conjugating antibodies with PEG which is attached to a radiometal chelator (diethylenetriaminpentaacetic acid (DTPA)).

The antibodies and antigen-binding fragments of the invention are, in an embodiment of the invention, conjugated with labels such as ^{99m}Tc, ⁹⁹Tc, ⁹⁰Y, ¹¹In, ³²P, ¹⁴C, ¹²⁵I, ³H, ¹³¹I, ¹²³I, ¹¹C, ¹⁵O, ¹³N, ¹⁸F, ³⁵S, ⁵¹Cr, ⁵⁷To, ²²⁶Ra, ⁶⁰Co, ⁵⁹Fe, ⁵⁷Se, ¹⁵²Eu, ⁶⁷CU, ²¹⁷Ci, ²¹¹At, ²¹²Pb, ⁴⁷Sc, ¹⁰⁹Pd, ²³⁴Th, and ⁴⁰K, ¹⁵⁷Gd, ⁵⁵Mn, ⁵²Tr and ⁵⁶Fe.

The antibodies and antigen-binding fragments of the invention may also be conjugated with fluorescent or chemilluminescent labels, including fluorophores such as rare earth chelates, fluorescein and its derivatives, rhodamine and its derivatives, isothiocyanate, phycoerythrin, phycocyanin, allophycocyanin, o-phthaladehyde, fluorescamine, ¹⁵²Eu, dansyl, umbelliferone, luciferin, luminal label, isoluminal label, an aromatic acridinium ester label, an imidazole label, an acridimium salt label, an oxalate ester label, an aequorin label, 2,3-dihydrophthalazinediones, biotin, avidin, peroxidase such as horseradish peroxidase, alkaline phosphatase (e.g., calf, shrimp or bacterial), spin labels and stable free radicals.

The antibodies and antigen-binding fragments of the invention may also be conjugated to a cytotoxic factor such as diptheria toxin, Pseudomonas aeruginosa exotoxin A chain, ricin A chain, abrin A chain, modeccin A chain, alpha-sarcin, Aleurites fordii proteins and compounds (e.g., fatty acids), dianthin proteins, Phytoiacca americana proteins PAPI, PAPII, and PAP-S, momordica charantia inhibitor, curcin, crotin, saponaria officinalis inhibitor, mitogellin, restrictocin, phenomycin, and enomycin.

Any method known in the art for conjugating the antibodies and antigen-binding fragments of the invention to the various moieties may be employed, including those methods described by Hunter, et al., (1962) Nature 144:945; David, et al., (1974) Biochemistry 13:1014; Pain, et al., (1981) J. Immunol. Meth. 40:219; and Nygren, J., (1982) Histochem. and Cytochem. 30:407. Methods for conjugating antibodies are conventional and very well known in the art.

Hybridomas expressing the 9H2 and 2C6 antibodies were deposited, under the Budapest Treaty, on May 10, 2007, with the American Type Culture Collection (ATCC); 10801 University Boulevard; Manassas, Va. 20110-2209. All restrictions on the accessibility of the deposited plasmids to the public will be irrevocably removed by the applicant upon the granting of a patent from this application. The ATCC deposit number for the 9H2.E12 hybridoma (expressing the 9H2 antibody) is PTA-8428 and the ATCC deposit number for the 2C6.B10 hybridoma (expressing the 2C6 antibody) is PTA-8429. Each of the isolated hybridomas form part of the present invention along with any composition comprising such a hybridoma (e.g., a hybridoma and a cellular growth medium). Also part of the present invention is any antibody which is produced by such hybridomas, any antigen-binding fragment thereof, any antibody of fragment comprising one or more of the CDRs (e.g., 3 light chain and/or 3 heavy chain) of such antibodies or fragments or any composition thereof, e.g., comprising a further therapeutic agent (as discussed herein). The scope of the present invention also includes a pharmaceutical composition comprising any such antibody or antigen-binding fragment thereof in association with a pharmaceutically acceptable carrier.

The present invention further comprises a method for generating an anti-IGF1R antibody of the present invention comprising culturing such a hybridoma under conditions suitable for expression of such an antibody and, optionally, isolation of such an antibody from the hybridoma. Any isolated antibody produced by such a process or any composition thereof forms part of the present invention. Any method of treating or preventing a medical disorder comprising an anti-IGF1R antibody, as discussed herein, comprising administering an antibody generated by a hybridoma with ATCC deposit number a PTA-8428 or PTA-8429 also forms part of the present invention.

In an embodiment of the invention, an antibody or antigen-binding fragment thereof binds “specifically” to an antigen, such as human IGF1R, if said binding exhibits a K_{D} of about 10⁻⁹M.

### Molecular Biology

In accordance with the present invention there may be employed conventional molecular biology, microbiology, and recombinant DNA techniques within the skill of the art. Such techniques are explained fully in the literature. See, e.g., Sambrook, Fritsch & Maniatis, Molecular Cloning: A Laboratory Manual, Second Edition (1989) Cold Spring Harbor Laboratory Press, Cold Spring Harbor, N.Y. (herein “Sambrook, et al., 1989”); DNA Cloning: A Practical Approach, Volumes I and II (D. N. Glover ed. 1985); Oligonucleotide Synthesis (M. J. Gait ed. 1984); Nucleic Acid Hybridization (B. D. Hames & S. J. Higgins eds. (1985)); Transcription And Translation (B. D. Hames & S. J. Higgins, eds. (1984)); Animal Cell Culture (R. I. Freshney, ed. (1986)); Immobilized Cells And Enzymes (IRL Press, (1986)); B. Perbal, A Practical Guide To Molecular Cloning (1984); F. M. Ausubel, et al. (eds.), Current Protocols in Molecular Biology, John Wiley & Sons, Inc. (1994).

A “polynucleotide”, “nucleic acid” or “nucleic acid molecule” includes the phosphate ester polymeric form of ribonucleosides (adenosine, guanosine, uridine or cytidine; “RNA molecules”) or deoxyribonucleosides (deoxyadenosine, deoxyguanosine, deoxythymidine, or deoxycytidine; “DNA molecules”), or any phosphoester analogs thereof, such as phosphorothioates and thioesters, in single stranded form, double-stranded form or otherwise.

A “polynucleotide sequence”, “nucleic acid sequence” or “nucleotide sequence” is a series of nucleotide bases (also called “nucleotides”) in a nucleic acid, such as DNA or RNA, and means any chain of two or more nucleotides.

A “coding sequence” or a sequence “encoding” an expression product, such as a RNA, polypeptide, protein, or enzyme, is a nucleotide sequence that, when expressed, results in production of the product.

The term “gene” includes a DNA sequence that codes for or corresponds to a particular sequence of ribonucleotides or amino acids which comprise all or part of one or more RNA molecules, proteins or enzymes, and may or may not include regulatory DNA sequences, such as promoter sequences, which determine, for example, the conditions under which the gene is expressed. Genes may be transcribed from DNA to RNA which may or may not be translated into an amino acid sequence.

“Amplification” of DNA as used herein includes the use of polymerase chain reaction (PCR) to increase the concentration of a particular DNA sequence within a mixture of DNA sequences. For a description of PCR see Saiki, et al., Science (1988) 239: 487. In a specific embodiment, the present invention includes a nucleic acid, which encodes anti-IGF1R antibody 2C6 or 9H2, anti-IGF1R antibody 2C6 or 9H2 heavy or light chain immunoglobulin, anti-IGFR1 antibody 2C6 or 9H2 heavy or light chain immunoglobulin variable region or an anti-IGFR1 antibody 2C6 or 9H2CDR (e.g., CDR-L1, CDR-L2, CDR-L3, CDR-H1, CDR-H2 or CDR-H3) which can be amplified by PCR.

As used herein, the term “oligonucleotide” includes a nucleic acid, generally of at least 10 (e.g., 10, 11, 12, 13 or 14), preferably at least 15 (e.g., 15, 16, 17, 18 or 19), and more preferably at least 20 nucleotides (e.g., 20, 21, 22, 23, 24, 25, 26, 27, 28, 29 or 30), preferably no more than 100 nucleotides (e.g., 40, 50, 60, 70, 80 or 90), that may be hybridizable to a genomic DNA molecule, a cDNA molecule, or an mRNA molecule encoding a gene, mRNA, cDNA, or other nucleic acid of interest. Oligonucleotides can be labeled, e.g., by incorporation of ³²P-nucleotides, ³H-nucleotides, ¹⁴C-nucleotides, ³⁵S-nucleotides or nucleotides to which a label, such as biotin, has been covalently conjugated. In one embodiment, a labeled oligonucleotide can be used as a probe to detect the presence of a nucleic acid. In another embodiment, oligonucleotides (one or both of which may be labeled) can be used as PCR primers, either for cloning full length or a fragment of the gene, or to detect the presence of nucleic acids. Generally, oligonucleotides are prepared synthetically, e.g., on a nucleic acid synthesizer.

The sequence of any nucleic acid may be sequenced by any method known in the art (e.g., chemical sequencing or enzymatic sequencing). “Chemical sequencing” of DNA includes methods such as that of Maxam and Gilbert (1977) (Proc. Natl. Acad. Sci. USA 74:560), in which DNA is randomly cleaved using individual base-specific reactions. “Enzymatic sequencing” of DNA may includes methods such as that of Sanger (Sanger, et al., (1977) Proc. Natl. Acad. Sci. USA 74:5463).

The nucleic acids herein may, in an embodiment of the invention, be flanked by natural regulatory (expression control) sequences, or may be associated with heterologous sequences, including promoters, internal ribosome entry sites (IRES) and other ribosome binding site sequences, enhancers, response elements, suppressors, signal sequences, polyadenylation sequences, introns, 5′- and 3′-non-coding regions, and the like.

A “promoter” or “promoter sequence” is, in an embodiment of the invention, a DNA regulatory region capable of binding an RNA polymerase in a cell (e.g., directly or through other promoter-bound proteins or substances) and initiating transcription of a coding sequence. A promoter sequence is, in general, bounded at its 3′ terminus by the transcription initiation site and extends upstream (5′ direction) to include the minimum number of bases or elements necessary to initiate transcription at any level. Within the promoter sequence may be found a transcription initiation site (conveniently defined, for example, by mapping with nuclease S1), as well as protein binding domains (consensus sequences) responsible for the binding of RNA polymerase. The promoter may be operably associated with other expression control sequences, including enhancer and repressor sequences or with a nucleic acid of the invention. Promoters which may be used to control gene expression include, but are not limited to, cytomegalovirus (CMV) promoter (U.S. Pat. Nos. 5,385,839 and 5,168,062), the SV40 early promoter region (Benoist, et al., (1981) Nature 290:304-310), the promoter contained in the 3′ long terminal repeat of Rous sarcoma virus (Yamamoto, et al., (1980) Cell 22:787-797), the herpes thymidine kinase promoter (Wagner, et al., (1981) Proc. Natl. Acad. Sci. USA 78:1441-1445), the regulatory sequences of the metallothionein gene (Brinster, et al., (1982) Nature 296:39-42); prokaryotic expression vectors such as the β-lactamase promoter (VIIIa-Komaroff, et al., (1978) Proc. Natl. Acad. Sci. USA 75:3727-3731), or the tac promoter (DeBoer, et al., (1983) Proc. Natl. Acad. Sci. USA 80:21-25); see also “Useful proteins from recombinant bacteria” in Scientific American (1980) 242:74-94; and promoter elements from yeast or other fungi such as the Gal 4 promoter, the ADC (alcohol dehydrogenase) promoter, PGK (phosphoglycerol kinase) promoter or the alkaline phosphatase promoter.

A coding sequence is “under the control of”, “functionally associated with” or “operably associated with” transcriptional or translational control sequences in a cell when the sequences direct RNA polymerase mediated transcription of the coding sequence into RNA, e.g., mRNA, which then may be trans-RNA spliced (if it contains introns) and, optionally, translated into a protein encoded by the coding sequence.

The terms “express” and “expression” include allowing or causing the information in a gene, RNA or DNA sequence to become manifest; for example, producing a protein by activating the cellular functions involved in transcription and translation of a corresponding gene. A DNA sequence is expressed in or by a cell to form an “expression product” such as an RNA (e.g., mRNA) or a protein (e.g., antibody 2C6 or 9H2 or an antigen-binding fragment thereof). The expression product itself may also be said to be “expressed” by the cell.

The terms “vector”, “cloning vector” and “expression vector” include a vehicle (e.g., a plasmid) by which a DNA or RNA sequence can be introduced into a host cell, so as to transform the host and, optionally, promote expression and/or replication of the introduced sequence.

The term “transfection” or “transformation” includes the introduction of a nucleic acid into a cell. These terms include the introduction of a nucleic acid encoding an anti-IGF1R antibody or fragment thereof into a cell. The introduced gene or sequence may be called a “clone”. A host cell that receives the introduced DNA or RNA has been “transformed” and is a “transformant” or a “clone”. The DNA or RNA introduced to a host cell can come from any source, including cells of the same genus or species as the host cell, or cells of a different genus or species.

The term “host cell” includes any cell of any organism that is selected, modified, transfected, transformed, grown, or used or manipulated in any way, for the production of a substance by the cell, for example the expression or replication, by the cell, of a gene, a DNA or RNA sequence, a protein or an enzyme.

The term “expression system” includes a host cell and compatible vector which, under suitable conditions, can express a protein or nucleic acid which is carried by the vector and introduced to the host cell. Common expression systems include E. coli host cells and plasmid vectors, insect host cells and Baculovirus vectors, and mammalian host cells and vectors. In a specific embodiment, IGF1R or an antibody and antigen-binding fragment of the invention may be expressed in human embryonic kidney cells (HEK293). Other suitable cells include CHO (chinese hamster ovary) cells, HeLa cells and NIH 3T3 cells and NSO cells (non-Ig-producing murine myeloma cell line). Nucleic acids encoding an antibody or antigen-binding fragment of the invention, sIGFR1 or IGFR1 may be expressed at high levels in an E. coli/T7 expression system as disclosed in U.S. Pat. Nos. 4,952,496, 5,693,489 and 5,869,320 and in Davanloo, P., et al., (1984) Proc. Natl. Acad. Sci. USA 81, 2035-2039; Studier, F. W., et al., (1986) J. Mol. Biol. 189: 113-130; Rosenberg, A. H., et al., (1987) Gene 56: 125-135; and Dunn, J. J., et al., (1988) Gene 68: 259 which are herein incorporated by reference.

The present invention contemplates any superficial or slight modification to the amino acid or nucleotide sequences which correspond to the 2C6 or 9H2 antibodies or antigen-binding fragments thereof of the invention. In particular, the present invention contemplates sequence conservative variants of the nucleic acids which encode the 2C6 or 9H2 antibodies or antigen-binding fragments thereof of the invention. “Sequence-conservative variants” of a polynucleotide sequence are those in which a change of one or more nucleotides in a given codon results in no alteration in the amino acid encoded at that position. Function-conservative variants of the antibodies of the invention are also contemplated by the present invention. “Function-conservative variants” are those in which one or more amino acid residues in a protein have been changed without altering the overall conformation and function of the polypeptide, including, but, by no means, limited to, replacement of an amino acid with one having similar properties. Amino acids with similar properties are well known in the art. For example, polar/hydrophilic amino acids which may be interchangeable include asparagine, glutamine, serine, cysteine, threonine, lysine, arginine, histidine, aspartic acid and glutamic acid; nonpolar/hydrophobic amino acids which may be interchangeable include glycine, alanine, valine, leucine, isoleucine, proline, tyrosine, phenylalanine, tryptophan and methionine; acidic amino acids which may be interchangeable include aspartic acid and glutamic acid and basic amino acids which may be interchangeable include histidine, lysine and arginine. Conservative substitutions of an amino acid sequence refer to those wherein an amino acid of one subtype (e.g., polar/hydrophilic) is replaced with another amino acid of the same subtype; and, in an embodiment of the invention, wherein the conservatively substituted polypeptide retains essentially the same level of biological activity (e.g., binding affinity of a substituted anti-IGF1R antibody for IGF1R).

The present invention includes nucleic acids encoding antibodies 2C6 and 9H2 and antigen-binding fragments thereof as well as nucleic acids which hybridize thereto. In an embodiment of the invention, the nucleic acids hybridize under low stringency conditions, more preferably under moderate stringency conditions and most preferably under high stringency conditions and, in an embodiment of the invention, exhibit IGF1R binding activity. A nucleic acid molecule is “hybridizable” to another nucleic acid molecule, such as a cDNA, genomic DNA, or RNA, when a single stranded form of the nucleic acid molecule can anneal to the other nucleic acid molecule under the appropriate conditions of temperature and solution ionic strength (see Sambrook, et al., supra). The conditions of temperature and ionic strength determine the “stringency” of the hybridization. Typical low stringency hybridization conditions include, in an embodiment of the invention, 55° C., 5×SSC, 0.1% SDS, 0.25% milk, and no formamide; or 30% formamide, 5×SSC, 0.5% SDS. Typical, moderate stringency hybridization conditions are similar to the low stringency conditions except the hybridization is carried out in 40% formamide, with 5× or 6×SSC. High stringency hybridization conditions are similar to low stringency conditions except the hybridization conditions are carried out in 50% formamide, 5× or 6×SSC and, optionally, at a higher temperature (e.g., 57° C., 59° C., 60° C., 62° C., 63° C., 65° C. or 68° C.). In general, SSC is 0.15M NaCl and 0.015M Na-citrate. Hybridization requires that the two nucleic acids contain complementary sequences, although, depending on the stringency of the hybridization, mismatches between bases are possible. The appropriate stringency for hybridizing nucleic acids depends on the length of the nucleic acids and the degree of complementation, variables well known in the art. The greater the degree of similarity or homology between two nucleotide sequences, the higher the stringency under which the nucleic acids may hybridize. For hybrids of greater than 100 nucleotides in length, equations for calculating the melting temperature have been derived (see Sambrook, et al., supra, 9.50-9.51). For hybridization with shorter nucleic acids, i.e., oligonucleotides, the position of mismatches becomes more important, and the length of the oligonucleotide determines its specificity (see Sambrook, et al., supra, 11.7-11.8). In an embodiment of the invention, a nucleic acid which hybridizes to a nucleic acid of the present invention exhibits one or more of the same activities and/or biochemical characteristics of an immunoglobulin chain of 9H2 or 2C6. For example, the nucleic acid exhibits an ability of bind to IGF1R or a fragment thereof, to inhibit IGF1R tyrosine kinase activity, to inhibit tumor cell growth (in vivo or in vitro), to inhibit anchorage indepent cell growth (in vivo or in vitro) or to inhibit binding between IGF1R or a fragment thereof and IGF-1 or IGF-2 to at least the same degree as 9H2 or 2C6 antibody or to a lesser degree (i.e., to any detectable degree).

Also included in the present invention are nucleic acids comprising nucleotide sequences and polypeptides comprising amino acid sequences which are at least about 70% identical, preferably at least about 80% identical, more preferably at least about 90% identical and most preferably at least about 95% identical (e.g., 95%, 96%, 97%, 98%, 99%, 100%) to the reference nucleotide and amino acid sequences of the present invention (i.e., as discussed herein) when the comparison is performed by a BLAST algorithm wherein the parameters of the algorithm are selected to give the largest match between the respective sequences over the entire length of the respective reference sequences. Polypeptides comprising amino acid sequences which are at least about 70% similar, preferably at least about 80% similar, more preferably at least about 90% similar and most preferably at least about 95% similar (e.g., 95%, 96%, 97%, 98%, 99%, 100%) to the reference amino acid sequences of the present invention when the comparison is performed with a BLAST algorithm wherein the parameters of the algorithm are selected to give the largest match between the respective sequences over the entire length of the respective reference sequences, are also included in the present invention.

The present invention also provides variants of 2C6 and 9H2 anti-IGF1R antibodies and antigen-binding fragments thereof, which bind specifically to human IGF1R (e.g., with essentially the same affinity as that of 2C6 or 9H2), but which include one or more conservative substitutions in the framework immunoglobulin variable region (i.e., outside the immunoglobulin CDRs). The present invention also includes 2C6 and 9H2 antibody and fragment variants wherein the framework comprises 90% or more (e.g., 91%, 92%, 93%, 94%, 95%, 96%, 97%, 98%, 99%) identity or similarity to that of the native 2C6 or 9H2 or any antigen-binding fragment thereof.

Sequence identity refers to exact matches between the nucleotides or amino acids of two sequences which are being compared. Sequence similarity refers to both exact matches between the amino acids of two polypeptides which are being compared in addition to matches between nonidentical, biochemically related amino acids. Biochemically related amino acids which share similar properties and may be interchangeable are discussed above.

The following references regarding the BLAST algorithm are herein

incorporated by reference: BLAST ALGORITHMS: Altschul, S. F., et al., (1990) J. Mol. Biol. 215:403-410; Gish, W., et al., (1993) Nature Genet. 3:266-272; Madden, T. L., et al., (1996) Meth. Enzymol. 266:131-141; Altschul, S. F., et al., (1997) Nucleic Acids Res. 25:3389-3402; Zhang, J., et al., (1997) Genome Res. 7:649-656; Wootton, J. C., et al., (1993) Comput. Chem. 17:149-163; Hancock, J. M. et al., (1994) Comput. Appl. Biosci. 10:67-70; ALIGNMENT SCORING SYSTEMS: Dayhoff, M. O., et al., “A model of evolutionary change in proteins.” in Atlas of Protein Sequence and Structure, (1978) vol. 5, suppl. 3. M. O. Dayhoff (ed.), pp. 345-352, Natl. Biomed. Res. Found., Washington, D.C.; Schwartz, R. M., et al., “Matrices for detecting distant relationships.” in Atlas of Protein Sequence and Structure, (1978) vol. 5, suppl. 3.” M. O. Dayhoff (ed.), pp. 353-358, Natl. Biomed. Res. Found., Washington, D.C.; Altschul, S. F., (1991) J. Mol. Biol. 219:555-565; States, D. J., et al., (1991) Methods 3:66-70; Henikoff, S., et al., (1992) Proc. Natl. Acad. Sci. USA 89:10915-10919; Altschul, S. F., et al., (1993) J. Mol. Evol. 36:290-300; ALIGNMENT STATISTICS: Karlin, S., et al., (1990) Proc. Natl. Acad. Sci. USA 87:2264-2268; Karlin, S., et al., (1993) Proc. Natl. Acad. Sci. USA 90:5873-5877; Dembo, A., et al., (1994) Ann. Prob. 22:2022-2039; and Altschul, S. F. “Evaluating the statistical significance of multiple distinct local alignments.” in Theoretical and Computational Methods in Genome Research (S. Suhai, ed.), (1997) pp. 1-14, Plenum, New York.

### Generation of Antibodies

Any suitable method for generating monoclonal antibodies may be used. The present invention includes both recombinant and non-recombinant methods of production, e.g., as discussed herein. Non-recombinant methods include immunization of animals and subsequent isolation of antibodies or splenocytes (e.g., followed by hybridoma production) from the immunized animal. For example, a recipient may be immunized with a linked or unlinked (e.g. naturally occurring) form of IGF1R, or a fragment thereof. Any suitable method of immunization can be used. Such methods can include adjuvants, other immunostimulants, repeated booster immunizations, and the use of one or more immunization routes.

Any form of the antigen can be used to generate the antibody that is sufficient to generate a biologically active antibody. Thus, the eliciting antigen may be a single epitope, multiple epitopes, or the entire protein alone or in combination with one or more immunogenicity enhancing agents known in the art. The eliciting antigen may be an isolated full-length protein, a cell surface protein (e.g., utilized by immunizing with cells transfected with at least a portion of the antigen), or a soluble protein (e.g., utilized by immunizing with only the extracellular domain portion of the protein). The antigen may be produced in a genetically modified cell. The DNA encoding the antigen may genomic or non-genomic (e.g., cDNA) and can encode at least a portion of the extracellular domain. As used herein, the term “portion” refers to the minimal number of amino acids or nucleic acids, as appropriate, to constitute an immunogenic epitope of the antigen of interest. Any genetic vectors suitable for transformation of the cells of interest may be employed, including but not limited to adenoviral vectors, plasmids, and non-viral vectors, such as cationic lipids.

Techniques for generating an antibody of the invention include selection of libraries of antibodies in phage or similar vectors. See, e.g., Huse et al., Science 246:1275-1281 (1989); and Ward et al., Nature 341:544-546 (1989).

In an embodiment of the invention, human monoclonal antibodies directed against IGF1R are generated using transgenic mice carrying parts of the human immune system rather than the mouse system. These transgenic mice, which may be referred to, herein, as “HuMAb” mice, contain human immunoglobulin gene miniloci that encodes unrearranged human heavy (μ and γ) and κ light chain immunoglobulin sequences, together with targeted mutations that inactivate the endogenous μ and κ chain loci (Lonberg, N., et al., (1994) Nature 368(6474): 856-859). Accordingly, the mice exhibit reduced expression of mouse IgM or κ, and in response to immunization, the introduced human heavy and light chain transgenes undergo class switching and somatic mutation to generate high affinity human IgGκ monoclonal antibodies (Lonberg, N., et al., (1994), supra; reviewed in Lonberg, N. (1994) Handbook of Experimental Pharmacology 113:49-101; Lonberg, N., et al., (1995) Intern. Rev. Immunol. 13:65-93, and Harding, F., et al., (1995) Ann. N.Y. Acad. Sci. 764:536-546). The preparation of HuMab mice is commonly known in the art and is described, for example, in Taylor, L., et al., (1992) Nucleic Acids Research 20:6287-6295; Chen, J., et al., (1993) International Immunology 5: 647-656; Tuaillon, et al., (1993) Proc. Natl. Acad. Sci. USA 90:3720-3724; Choi, et al., (1993) Nature Genetics 4:117-123; Chen, J., et al., (1993) EMBO J. 12: 821-830; Tuaillon, et al., (1994) J. Immunol. 152:2912-2920; Lonberg, et al., (1994) Nature 368(6474): 856-859; Lonberg, N. (1994) Handbook of Experimental Pharmacology 113:49-101; Taylor, L., et al., (1994) International Immunology 6: 579-591; Lonberg, N., et al., (1995) Intern. Rev. Immunol. Vol. 13: 65-93; Harding, F., et al., (1995) Ann. N.Y. Acad. Sci. 764:536-546; Fishwild, D., et al., (1996) Nature Biotechnology 14: 845-851 and Harding, et al., (1995) Annals NY Acad. Sci. 764:536-546; the contents of all of which are hereby incorporated by reference in their entirety. See further, U.S. Pat. Nos. 5,545,806; 5,569,825; 5,625,126; 5,633,425; 5,789,650; 5,877,397; 5,661,016; 5,814,318; 5,874,299; 5,770,429 and 5,545,807; and International Patent Application Publication Nos. WO 98/24884; WO 94/25585; WO 93/12227; WO 92/22645 and WO 92/03918 the disclosures of all of which are hereby incorporated by reference in their entity. The use of HuMAb mice is commercially available from Medarex, Inc. (Princeton, N.J.).

To generate fully human, monoclonal antibodies to IGF1R, HuMab mice can be immunized with an antigenic IGF1R polypeptide, as described by Lonberg, N., et al., (1994) Nature 368(6474): 856-859; Fishwild, D., et al., (1996) Nature Biotechnology 14: 845-851 and WO 98/24884. In an embodiment of the invention, the mice will be 6-16 weeks of age upon the first immunization. For example, a purified preparation of IGF1R or sIGF1R can be used to immunize the HuMab mice intraperitoneally. The mice can also be immunized with whole HEK293 cells which are stably transformed or transfected with an IGF1R gene.

In general, HuMAb transgenic mice respond well when initially immunized intraperitoneally (i.p.) with antigen in complete Freund's adjuvant, followed by every other week IP immunizations (usually, up to a total of 6) with antigen in incomplete Freund's adjuvant. Mice can be immunized, first, with cells expressing IGF1R (e.g., stably transformed HEK293 cells), then with a soluble fragment of IGF1R and continually receive alternating immunizations with the two antigens. The immune response can be monitored over the course of the immunization protocol with plasma samples being obtained by retroorbital bleeds. The plasma can be screened for the presence of anti-IGF1R antibodies, for example by ELISA, and mice with sufficient titers of immunoglobulin can be used for fusions. Mice can be boosted intravenously with antigen 3 days before sacrifice and removal of the spleen. Several mice can be immunized for each antigen.

Hybridoma cells which produce the monoclonal, fully human anti-IGF1R antibodies may be produced by methods which are commonly known in the art. These methods include, but are not limited to, the hybridoma technique originally developed by Kohler, et al., (1975) (Nature 256:495-497), as well as the trioma technique (Hering, et al., (1988) Biomed. Biochim. Acta. 47:211-216 and Hagiwara, et al., (1993) Hum. Antibod. Hybridomas 4:15), the human B-cell hybridoma technique (Kozbor, et al., (1983) Immunology Today 4:72 and Cote, et al., (1983) Proc. Natl. Acad. Sci. U.S.A 80:2026-2030), and the EBV-hybridoma technique (Cole, et al., in Monoclonal Antibodies and Cancer Therapy, Alan R. Liss, Inc., pp. 77-96, 1985). In an embodiment of the invention, mouse splenocytes are isolated and fused with PEG to a mouse myeloma cell line based upon standard protocols. The resulting hybridomas are then screened for the production of antigen-specific antibodies. For example, single cell suspensions of splenic lymphocytes from immunized mice may by fused to one-sixth the number of P3X63-Ag8.653 nonsecreting mouse myeloma cells (ATCC, CRL 1580) with 50% PEG. Cells are plated at approximately 2×10⁵ cells/mL in a flat bottom microtiter plate, followed by a two week incubation in selective medium containing 20% fetal Clone Serum, 18% “653” conditioned media, 5% origen (IGEN), 4 mM L-glutamine, 1 mM L-glutamine, 1 mM sodium pyruvate, 5 mM HEPES, 0.055 mM 2-mercaptoethanol, 50 units/ml penicillin, 50 mg/ml streptomycin, 50 mg/ml gentamycin and 1×HAT (Sigma; the HAT is added 24 hours after the fusion). After two weeks, cells are cultured in medium in which the HAT is replaced with HT. Individual wells are then screened by ELISA for identification of human anti-IGF1R monoclonal IgG antibodies. Once extensive hybridoma growth occurs, medium can be observed usually after 10-14 days. The antibody secreting hybridomas may be replated, screened again, and if still positive for human IgG, anti-IGF1R monoclonal antibodies, can be subcloned at least twice by limiting dilution. The stable subclones may then be cultured in vitro to generate small amounts of antibody in tissue culture medium for characterization.

In general, for recombinant production of an immunoglobulin chain, a nucleic acid encoding it is isolated and inserted into a replicable vector for further cloning (amplification of the DNA) and/or for expression. DNA encoding the chain is readily isolated and sequenced using conventional procedures (e.g., by using oligonucleotide probes that are capable of binding specifically to genes encoding the heavy and light chains of the antibody). Many vectors are available. The vector components generally include, but are not limited to, one or more of the following: a signal sequence, an origin of replication, one or more marker genes, an enhancer element, a promoter, and a transcription termination sequence.

Recombinant immunoglobulins may be produced, e.g., by the method of Cabilly U.S. Pat. No. 4,816,567; and Queen et al. (1989) Proc. Nat'l Acad. Sci. USA 86:10029-10033; or made in transgenic mice, see Mendez et al. (1997) Nature Genetics 15:146-156. A recombinant method may comprise preparing a DNA sequence encoding an immunoglobulin heavy or light chain having specificity for a particular antigen; inserting the sequence into a replicable expression vector operably linked to a suitable promoter compatible with a host cell (e.g., bacterial cell such as E. coli or a mammalian cell); transforming the host cell with the vector; culturing the host cell; and recovering the heavy or light chain from the host cell culture.

In one embodiment of the invention, the antibodies or fragments of the present invention are produced in yeast according to the methods described in published international patent application no. WO2005/040395. Briefly, vectors encoding the individual light or heavy chains of an antibody of interest are introduced into different yeast haploid cells, e.g. different mating types of the yeast Pichia pastoris, which yeast haploid cells are optionally complementary auxotrophs. The transformed haploid yeast cells can then be mated or fused to give a diploid yeast cell capable of producing both the heavy and the light chains. The diploid strain is then able to secret the fully assembled and biologically active antibody. The relative expression levels of the two chains can be optimized, for example, by using vectors with different copy numbers, using transcriptional promoters of different strengths, or inducing expression from inducible promoters driving transcription of the genes encoding one or both chains.

In an embodiment of the present invention, the respective heavy and light chains of a plurality of different anti-IGF1R antibodies (the “original” antibodies) are introduced into yeast haploid cells to create a library of haploid yeast strains of one mating type expressing a plurality of light chains, and a library of haploid yeast strains of a different mating type expressing a plurality of heavy chains. These libraries of haploid strains can be mated (or fused as spheroplasts) to produce a series of dipoid yeast cells expressing a combinatorial library of antibodies comprised of the various possible permutations of light and heavy chains. The combinatorial library of antibodies can then be screened to determine whether any of the antibodies has properties that are superior (e.g., higher affinity for IGF1R) to those of the original antibodies.

In an embodiment of the invention, immunoglobulin chains of 2C6 and/or 9H2 are generated by any of the recombinant immunoglobulin production methods set forth in published U.S. patent application no. US2005/0176099 to D. Saha. Such methods and the plasmids set forth therein containing one or more light and/or heavy chain immunoglobulins of 2C6 or 9H2 form part of the present invention. For example, an embodiment of the invention comprises introduction of 2C6 or 9H2 light and heavy chain immunoglobulin into universal transfer vectors (pULLS or pUHLS) and transfer of the chains from the universal transfer vectors into an amplifiable vector, such as pXBLS (or a derivative thereof), followed by introduction of the amplifiable vector into a cell for expression and isolation.

### Further Chemotherapeutics

The scope of the present invention comprises compositions comprising one or more anti-IGF1R antibodies or antigen-binding fragments thereof of the invention optionally in association with one or more further chemotherapeutic agents along with methods for treating any medical disorder or disease set forth herein by administration of such a composition. A further chemotherapeutic agent comprises any agent that elicits a beneficial physiological response in an individual to which it is administered; for example, wherein the agent alleviates or eliminates disease symptoms or causes within the subject to which it is administered. A further chemotherapeutic agent includes any anti-cancer chemotherapeutic agent. An anti-cancer therapeutic agent is any agent that, for example, alleviates or eliminates symptoms or causes of cancer in the subject to which it is administered.

The present invention includes within its scope compositions comprising any anti-IGF1R antibody or antigen-binding fragment thereof in association with a further chemotherapeutic agent set forth herein and pharmaceutical compositions thereof.

In an embodiment of the invention, the further chemotherapeutic agent is a FLT-3 inhibitor, a VEGFR inhibitor, an EGFR TK inhibitor, an aurora kinase inhibitor, a PIK-1 modulator, a Bcl-2 inhibitor, an HDAC inhibitor, a c-MET inhibitor, a PARP inhibitor, a Cdk inhibitor, an EGFR TK inhibitor, an IGFR-TK inhibitor, an anti-HGF antibody, a PI3 kinase inhibitors, an AKT inhibitor, a JAK/STAT inhibitor, a checkpoint-1 or 2 inhibitor, a focal adhesion kinase inhibitor, a Map kinase kinase (mek) inhibitor or a VEGF trap antibody.

The present invention includes embodiments comprising an anti-IGF1R antibody of the present invention, or an antigen-binding fragment thereof in association with another anti-IGF1R antibody or antigen-binding fragment including, e.g., 15H12/19D12 LCC, 15H12/19D12 LCD, 15H12/19D12 LCE, 15H12/19D12 LCF, 15H12/19D12 HCA or 15H12/19D12 HCB or any combination thereof (e.g., a full antibody comprising LCB and HCB; LCC and HCA; or LCF and HCA).

Dotted, underscored type encodes the signal peptide. Solid underscored type encodes the CDRs. Plain type encodes the framework regions. Most preferably, the antibody chains are mature fragments which lack the signal peptide.

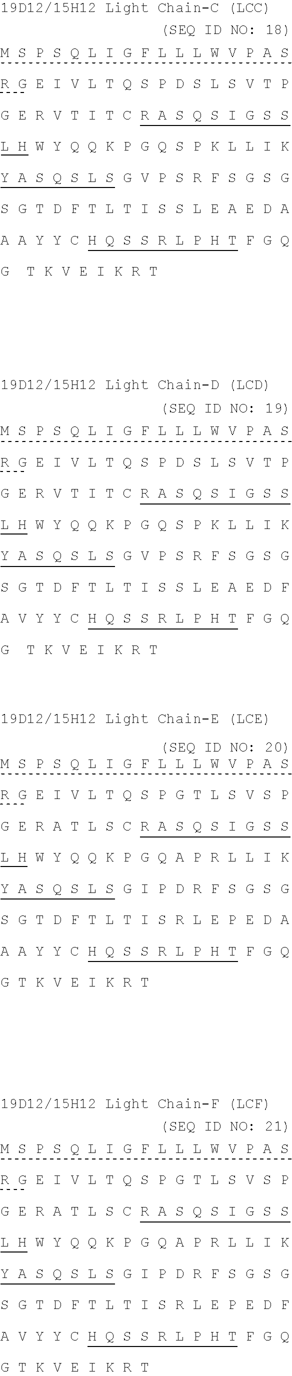

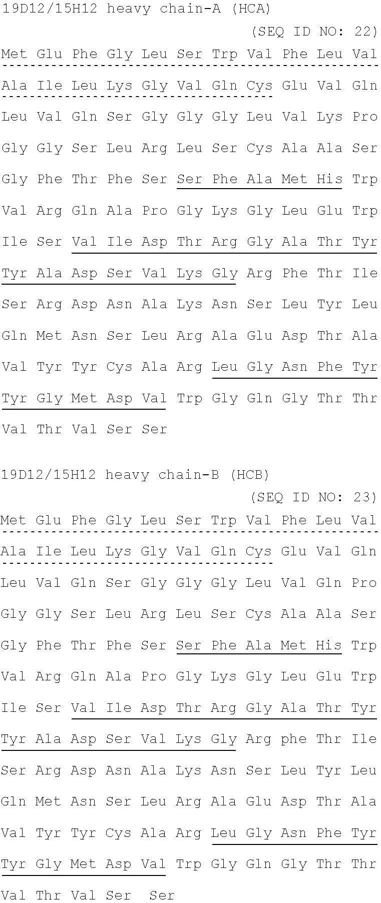

See international application publication no. WO03/100008.

In an embodiment of the invention, an antibody or antigen-binding fragment thereof of the invention is provided in association with erlotinib, dasatanib, nilotinib, decatanib, panitumumab, amrubicin, oregovomab, Lep-etu, nolatrexed, azd2171, batabulin, ofatumumab, zanolimumab, edotecarin, tetrandrine, rubitecan, tesmilifene, oblimersen, ticilimumab, ipilimumab, gossypol, Bio 111, 131-I-TM-601, ALT-110, BIO 140, CC 8490, cilengitide, gimatecan, IL13-PE38QQR, INO 1001, IPdR, KRX-0402, lucanthone, LY 317615, neuradiab, vitespan, Rta 744, Sdx 102, talampanel, atrasentan, Xr 311, everolimus, trabectedin, abraxane, TLK 286, AV-299, DN-101, pazopanib, GSK690693, RTA 744, ON 0910.Na, AZD 6244 (ARRY-142886), AMN-107, TKI-258, GSK461364, AZD 1152, enzastaurin, vandetanib, ARQ-197, MK-0457, MLN8054, PHA-739358, R-763 or AT-9263.

Abraxane is an injectable suspension of paclitaxel protein-bound particles comprising an albumin-bound form of paclitaxel with a mean particle size of approximately 130 nanometers. Abraxane is supplied as a white to yellow, sterile, lyophilized powder for reconstitution with 20 mL of 0.9% Sodium Chloride Injection, USP prior to intravenous infusion. Each single-use vial contains 100 mg of paclitaxel and approximately 900 mg of human albumin. Each milliliter (mL) of reconstituted suspension contains 5 mg paclitaxel. Abraxane is free of solvents and is free of cremophor (polyoxyethylated castor oil).

In an embodiment of the invention, an antibody or antigen-binding fragment thereof of the invention is provided in association with romidepsin (FK-228;

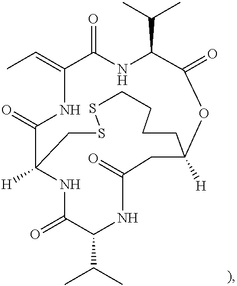

### ADS-100380,

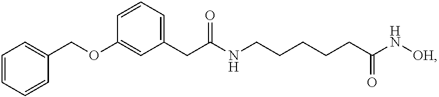

### CG-781

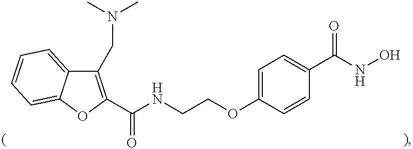

### CG-1521

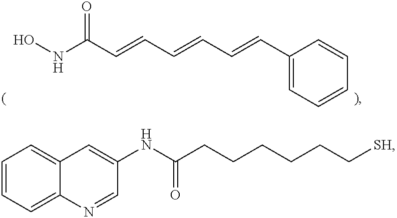

### SB-556629

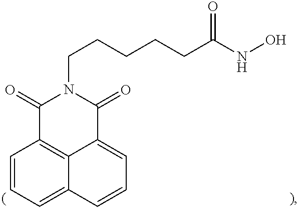

chlamydocin

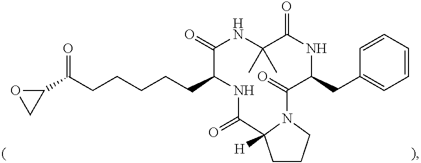

### UNJ-16241199

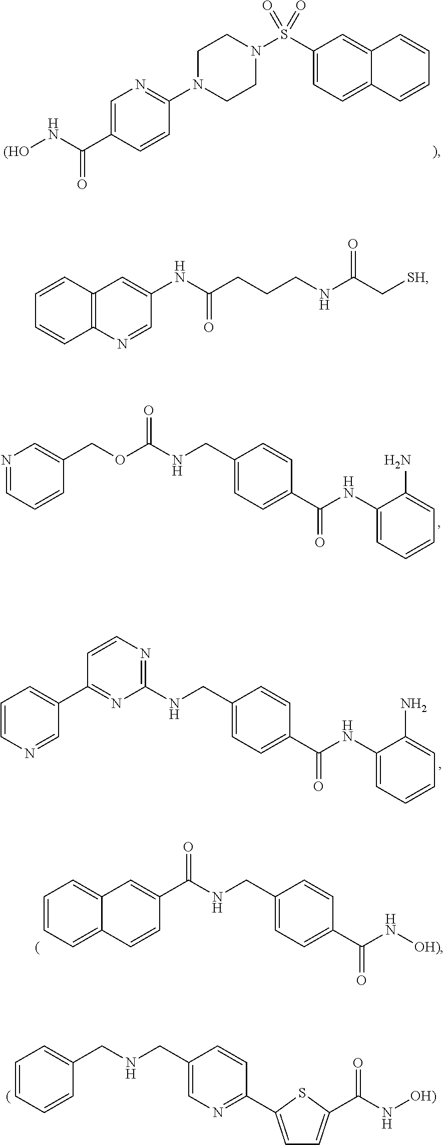

or vorinostat (SAHA;

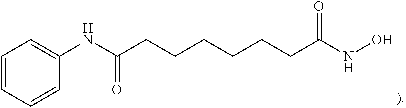

In an embodiment of the invention, an antibody or antigen-binding fragment thereof of the invention is provided in association with etoposide (VP-16;

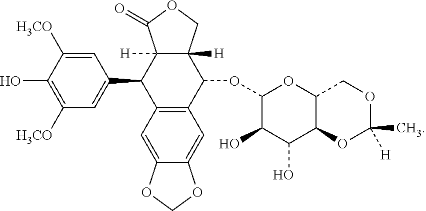

In an embodiment of the invention, an antibody or antigen-binding fragment thereof of the invention is provided in association with gemcitabine

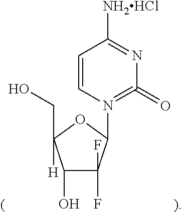

In an embodiment of the invention, an antibody or antigen-binding fragment thereof of the invention is provided in association with any compound disclosed in published U.S. patent application no. U.S. 2004/0209878A1 (e.g., comprising a core structure represented by

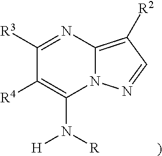

or doxorubicin

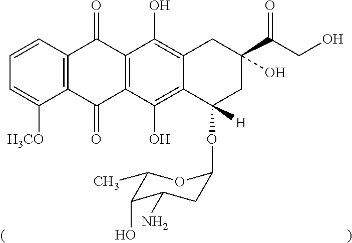

including Caelyx or Doxil® (doxorubicin HCl liposome injection; Ortho Biotech Products L.P; Raritan, N.J.). Doxil® comprises doxorubicin in STEALTH® liposome carriers which are composed of N-(carbonyl-methoxypolyethylene glycol 2000)-1,2-distearoyl-sn-glycero-3-phosphoethanolamine sodium salt (MPEG-DSPE); fully hydrogenated soy phosphatidylcholine (HSPC), and cholesterol.

In an embodiment of the invention, an antibody or antigen-binding fragment thereof of the invention is provided in association with 5′-deoxy-5-fluorouridine

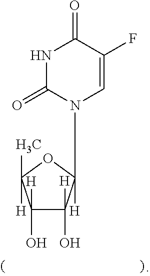

In an embodiment of the invention, an antibody or antigen-binding fragment thereof of the invention is provided in association with vincristine

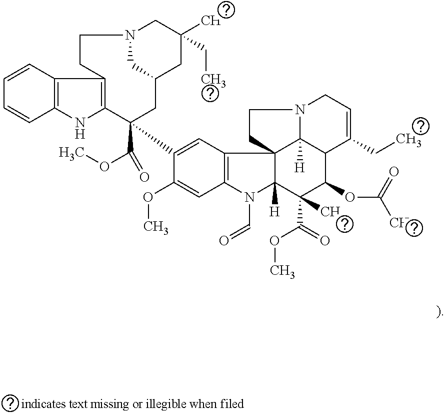

In an embodiment of the invention, an antibody or antigen-binding fragment thereof of the invention is provided in association with temozolomide

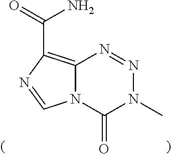

any CDK inhibitor such as ZK-304709, Seliciclib (R-roscovitine)

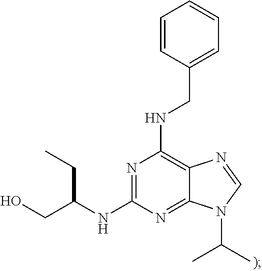

any MEK inhibitor such as PD0325901

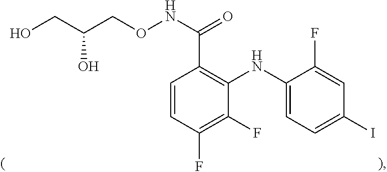

AZD-6244; capecitabine (5′-deoxy-5-fluoro-N-[(pentyloxy)carbonyl]-cytidine); or L-Glutamic acid, N-[4-[2-(2-amino-4,7-dihydro-4-oxo-1H-pyrrolo[2,3-d]pyrimidin-5-yl)ethyl]benzoyl]-, disodium salt, heptahydrate

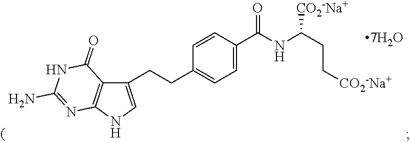

Pemetrexed disodium heptahydrate).

In an embodiment of the invention, an antibody or antigen-binding fragment thereof of the invention is provided in association with camptothecin

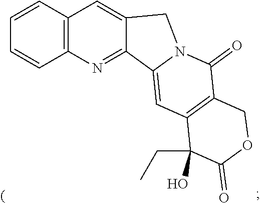

Stork et al., J. Am. Chem. Soc. 93(16): 4074-4075 (1971); Beisler et al., J. Med. Chem. 14(11): 1116-1117 (1962)), irinotecan

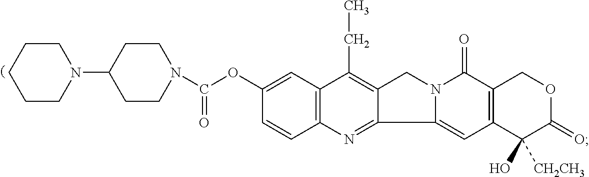

sold as Camptosar®; Pharmacia & Upjohn Co.; Kalamazoo, Mich.); a combination of irinotecan, 5-fluorouracil and leucovorin; or PEG-labeled irinotecan.

In an embodiment of the invention, an antibody or antigen-binding fragment thereof of the invention is provided in association with the FOLFOX regimen (oxaliplatin

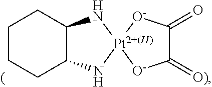

together with infusional fluorouracil

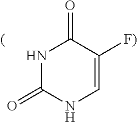

and folinic acid

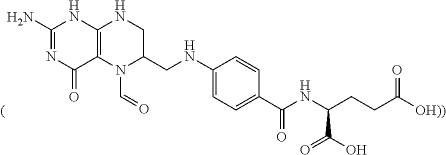

(Chaouche et al., Am. J. Clin. Oncol. 23(3):288-289 (2000); de Gramont et al., J. Clin. Oncol. 18(16):2938-2947 (2000)).

In an embodiment of the invention, an antibody or antigen-binding fragment thereof of the invention is provided in association with an antiestrogen such as

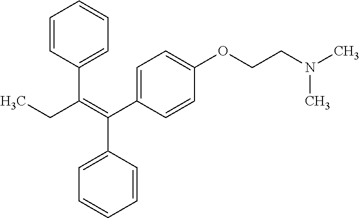

(tamoxifen; sold as Nolvadex® by AstraZeneca Pharmaceuticals LP; Wilmington, Del.) or

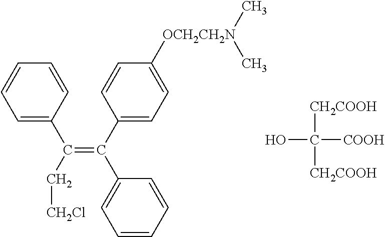

(toremifene citrate; sold as Fareston® by Shire US, Inc.; Florence, Ky.).

In an embodiment of the invention, an antibody or antigen-binding fragment thereof of the invention is provided in association with an aromatase inhibitor such as

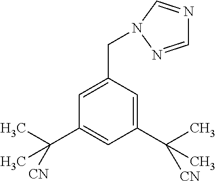

(anastrazole; sold as Arimidex® by AstraZeneca Pharmaceuticals LP; Wilmington, Del.),

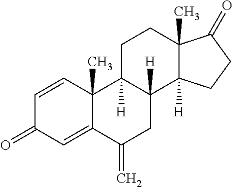

(exemestane; sold as Aromasin® by Pharmacia Corporation; Kalamazoo, Mich.) or

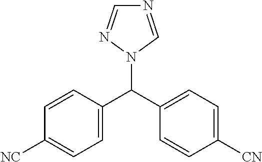

(letrozole; sold as Femara® by Novartis Pharmaceuticals Corporation; East Hanover, N.J.).

In an embodiment of the invention, an antibody or antigen-binding fragment thereof of the invention is provided in association with an estrogen such as DES(diethylstilbestrol),

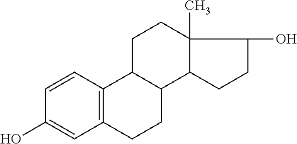

(estradiol; sold as Estrol® by Warner Chilcott, Inc.; Rockaway, N.J.) or conjugated estrogens (sold as Premarin® by Wyeth Pharmaceuticals Inc.; Philadelphia, Pa.).

In an embodiment of the invention, an antibody or antigen-binding fragment thereof of the invention is provided in association with anti-angiogenesis agents including bevacizumab (Avastin™, Genentech; San Francisco, Calif.), the anti-VEGFR-2 antibody IMC-1C11, other VEGFR inhibitors such as: CHIR-258

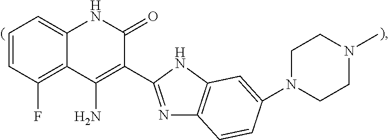

any of the inhibitors set forth in WO2004/13145 (e.g., comprising the core structural formula:

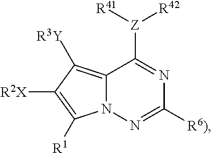

WO2004/09542 (e.g., comprising the core structural formula:

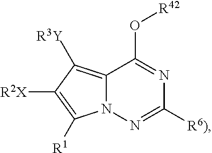

WO00/71129 (e.g., comprising the core structural formula:

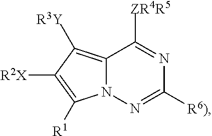

WO2004/09601 (e.g., comprising the core structural formula:

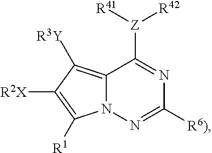

WO2004/01059 (e.g., comprising the core structural formula:

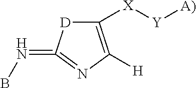

WO01/29025 (e.g., comprising the core structural formula:

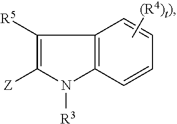

WO02/32861 (e.g., comprising the core structural formula:

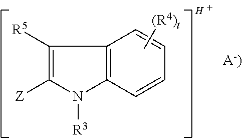

or set forth in WO03/88900 (e.g., comprising the core structural formula

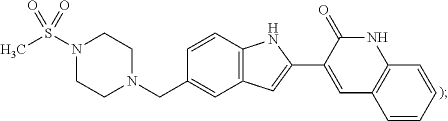

3-[5-(methylsulfonylpiperadinemethyl)-indolyl]-quinolone; Vatalanib

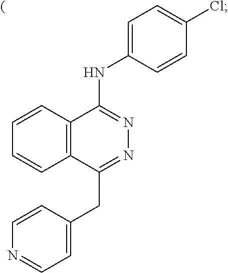

### PTK/ZK; CPG-79787; ZK-222584), AG-013736

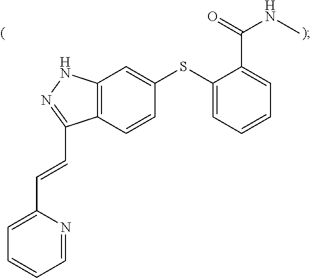

and the VEGF trap (AVE-0005), a soluble decoy receptor comprising portions of VEGF receptors 1 and 2.

In an embodiment of the invention, an antibody or antigen-binding fragment thereof of the invention is provided in association with a LHRH (Lutenizing hormone-releasing hormone) agonist such as the acetate salt of [D-Ser(Bu t)6, Azgly 10] (pyro-Glu-His-Trp-Ser-Tyr-D-Ser(Bu t)-Leu-Arg-Pro-Azgly-NH2 acetate [C₅₉H₈₄N₁₈O₁₄.(C₂H4O₂)ₓ where x=1 to 2.4];

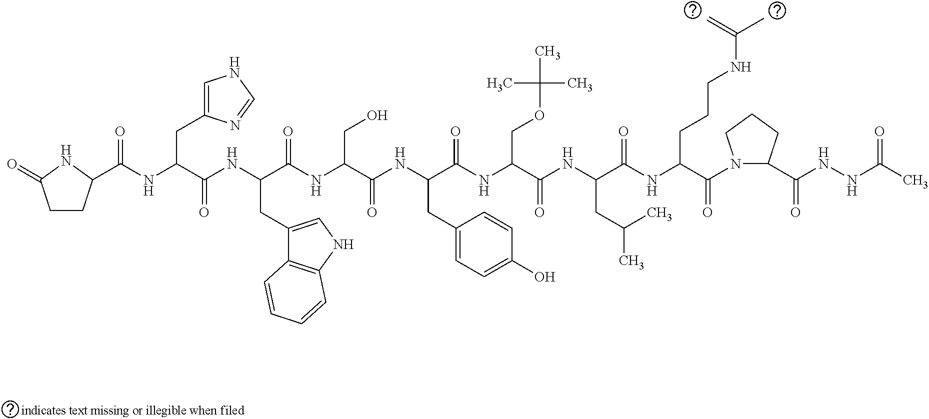

(goserelin acetate; sold as Zoladex® by AstraZeneca UK Limited; Macclesfield, England),

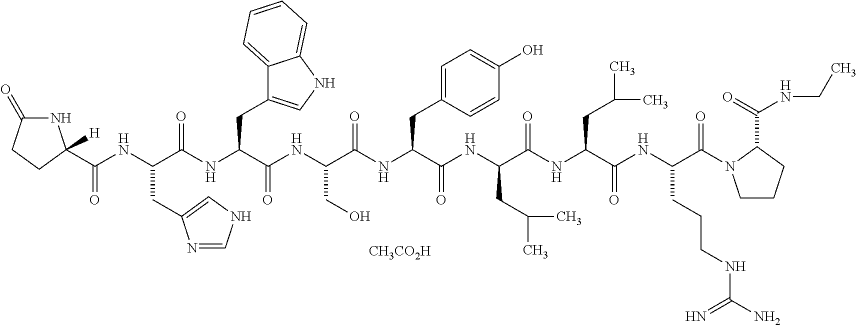

(leuprolide acetate; sold as Eligard® by Sanofi-Synthelabo Inc.; New York, N.Y.) or

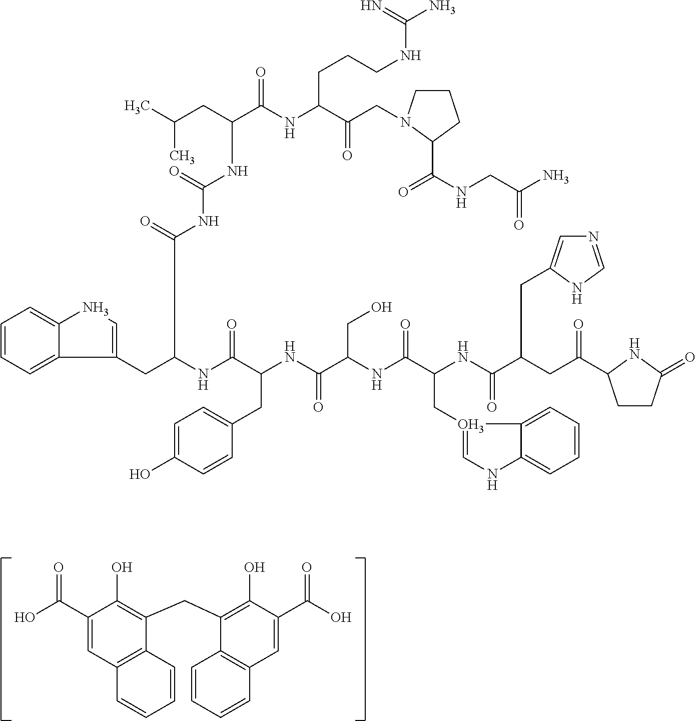

(triptorelin pamoate; sold as Trelstar® by Pharmacia Company, Kalamazoo, Mich.).

In an embodiment of the invention, an antibody or antigen-binding fragment thereof of the invention is provided in association with sunitinib or sunitinib malate

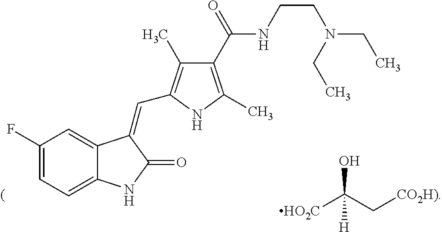

In an embodiment of the invention, an antibody or antigen-binding fragment thereof of the invention is provided in association with a progestational agent such as

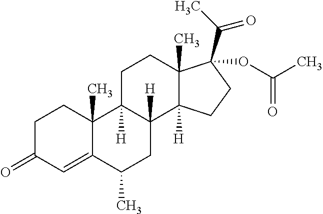

(medroxyprogesterone acetate; sold as Provera® by Pharmacia & Upjohn Co.; Kalamazoo, Mich.),

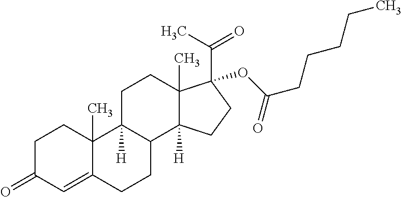

(hydroxyprogesterone caproate; 17-((1-Oxohexyl)oxy)pregn-4-ene-3,20-dione;), megestrol acetate or progestins.

In an embodiment of the invention, an antibody or antigen-binding fragment thereof of the invention is provided in association with selective estrogen receptor modulator (SERM) such as

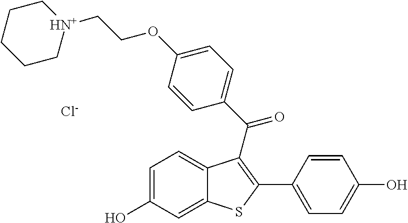

(raloxifene; sold as Evista® by Eli Lilly and Company; Indianapolis, Ind.).

In an embodiment of the invention, an antibody or antigen-binding fragment thereof of the invention is provided in association with an anti-androgen including, but not limited to:

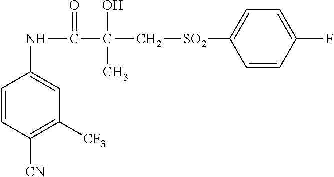

(bicalutamide; sold at CASODEX® by AstraZeneca Pharmaceuticals LP; Wilmington, Del.);

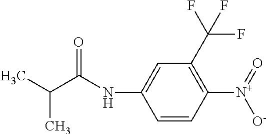

(flutamide; 2-methyl-N-[4-nitro-3 (trifluoromethyl)phenyl] propanamide; sold as Eulexin® by Schering Corporation; Kenilworth, N.J.);

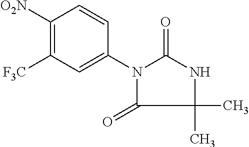

(nilutamide; sold as Nilandron® by Aventis Pharmaceuticals Inc.; Kansas City, Mo.) and

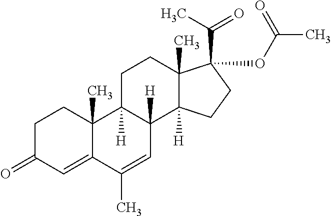

(Megestrol acetate; sold as Megace® by Bristol-Myers Squibb).

In an embodiment of the invention, an antibody or antigen-binding fragment thereof of the invention is provided in association with one or more inhibitors which antagonize the action of the EGF Receptor or HER2, including, but not limited to, CP-724714

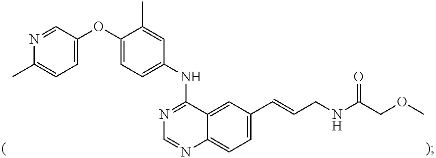

### TAK-165

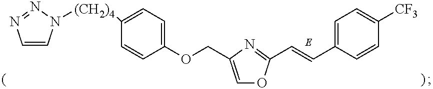

### HKI-272

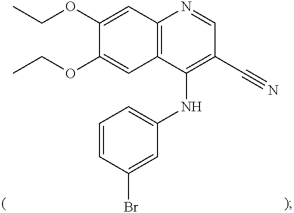

### OSI-774

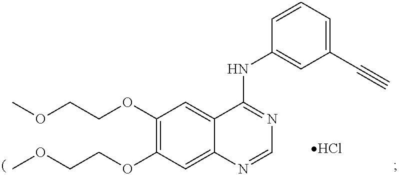

erlotinib, Hidalgo et al., J. Clin. Oncol. 19(13): 3267-3279 (2001)), Lapatanib

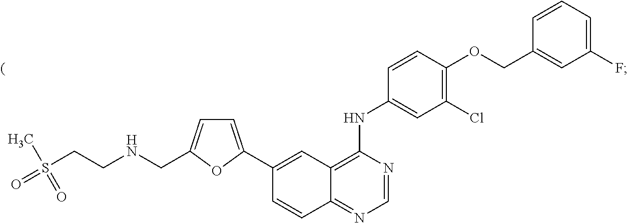

GW2016; Rusnak et al., Molecular Cancer Therapeutics 1:85-94 (2001); N-{3-Chloro-4-[(3-fluorobenzyl)oxy]phenyl}-6-[5-({[2-(methylsulfonyl)ethyl]amino}methyl)-2-furyl]-4-quinazolinamine; PCT Application No. WO99/35146), Canertinib (CI-1033;

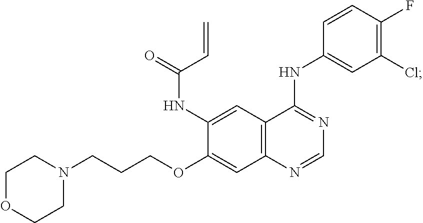

Erlichman et al., Cancer Res. 61(2):739-48 (2001); Smaill et al., J. Med. Chem. 43(7):1380-97 (2000)), ABX-EGF antibody (Abgenix, Inc.; Freemont, Calif.; Yang et al., Cancer Res. 59(6):1236-43 (1999); Yang et al., Crit. Rev Oncol Hematol. 38(1):17-23 (2001)), erbitux (U.S. Pat. No. 6,217,866; IMC-C225, cetuximab; Imclone; New York, N.Y.), EKB-569

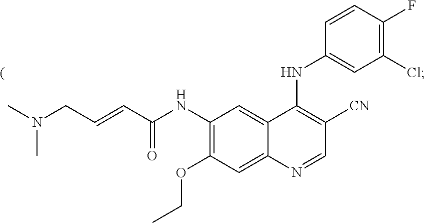

Wissner et al., J. Med. Chem. 46(1): 49-63 (2003)), PKI-166

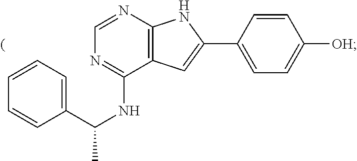

CGP-75166), GW-572016, any anti-EGFR antibody and any anti-HER2 antibody.

In an embodiment of the invention, an antibody or antigen-binding fragment thereof of the invention is provided in association with:

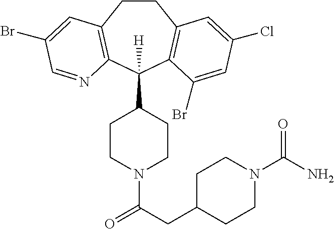

(lonafarnib; Sarasar™; Schering-Plough; Kenilworth, N.J.). In another embodiment, one of the following FPT inhibitors is provided in association with an antibody or antigen-binding fragment thereof of the invention:

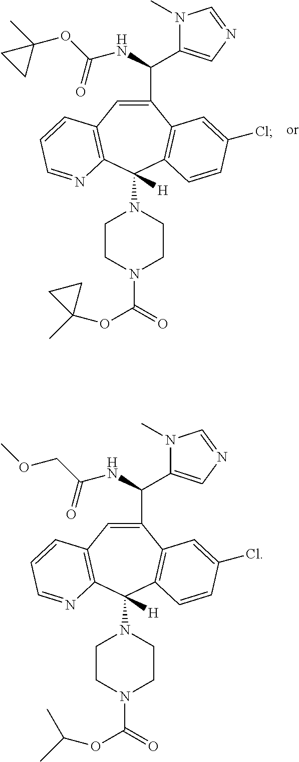

Other FPT inhibitors, that can be provided in association with an antibody or antigen-binding fragment thereof of the invention, include BMS-214662

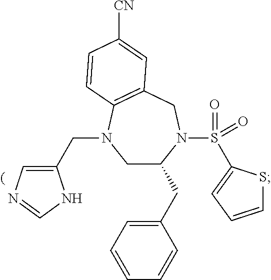

Hunt et al., J. Med. Chem. 43(20):3587-95 (2000); Dancey et al., Curr. Pharm. Des. 8:2259-2267 (2002); (R)-7-cyano-2,3,4,5-tetrahydro-1-(1H-imidazol-4-ylmethyl)-3-(phenylmethyl)-4-(2-thienylsulfonyl)-1H-1,4-benzodiazepine)) and R155777 (tipifarnib; Garner et al., Drug Metab. Dispos. 30(7):823-30 (2002); Dancey et al., Curr. Pharm. Des. 8:2259-2267 (2002); (B)-6-[amino(4-chlorophenyl)(1-methyl-1H-imidazol-5-yl)-methyl]-4-(3-chlorophenyl)-1-methyl-2(1H)-quinolinone];

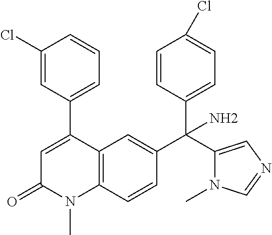

sold as Zarnestra™, Johnson & Johnson; New Brunswick, N.J.).

In an embodiment of the invention, an antibody or antigen-binding fragment thereof of the invention is provided in association with

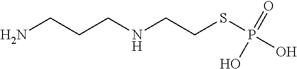

### (Amifostine);

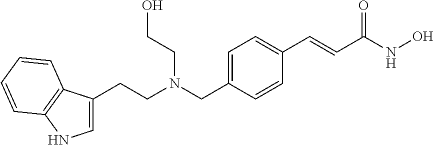

### (NVP-LAQ824; Atadja et al., Cancer Research 64: 689-695 (2004)),

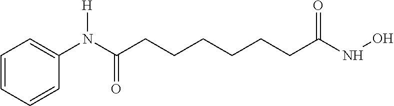

(suberoyl analide hydroxamic acid),

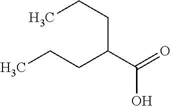

(Valproic acid; Michaelis et al., Mol. Pharmacol. 65:520-527 (2004)),

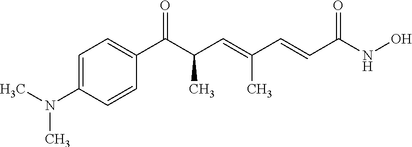

(trichostatin A),

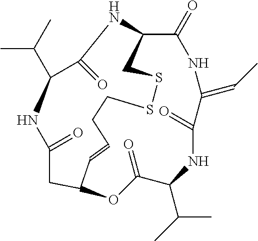

### (FK-228; Furumai et al., Cancer Research 62: 4916-4921 (2002)),

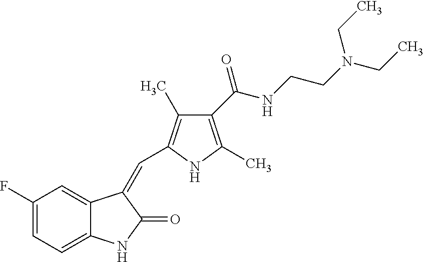

(SU11248; Mendel et al., Clin. Cancer Res. 9(1):327-37 (2003)),

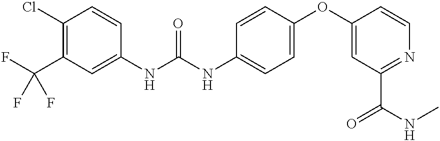

(BAY43-9006; sorafenib),

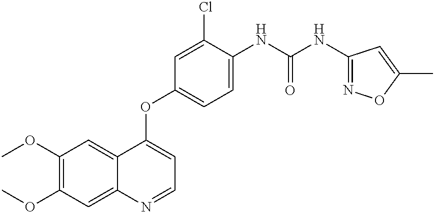

### (KRN951),

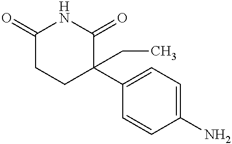

### (Aminoglutethimide);

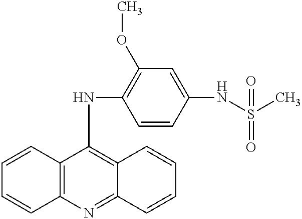

### (Amsacrine);

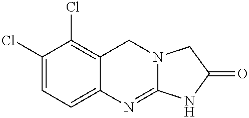

### (Anagrelide);

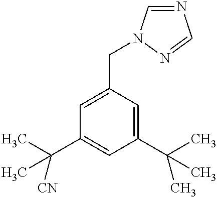

(Anastrozole; sold as Arimidex by AstraZeneca Pharmaceuticals LP; Wilmington, Del.); Asparaginase; Bacillus Calmette-Guerin (BCG) vaccine (Gamido et al., Cytobios. 90(360):47-65 (1997));

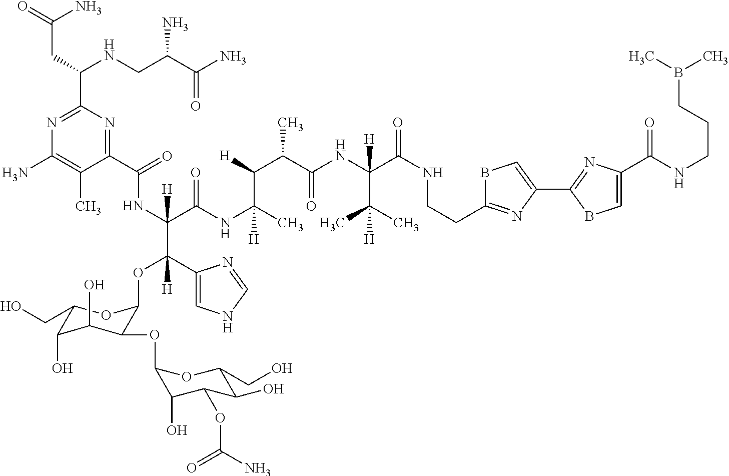

### (Bleomycin);

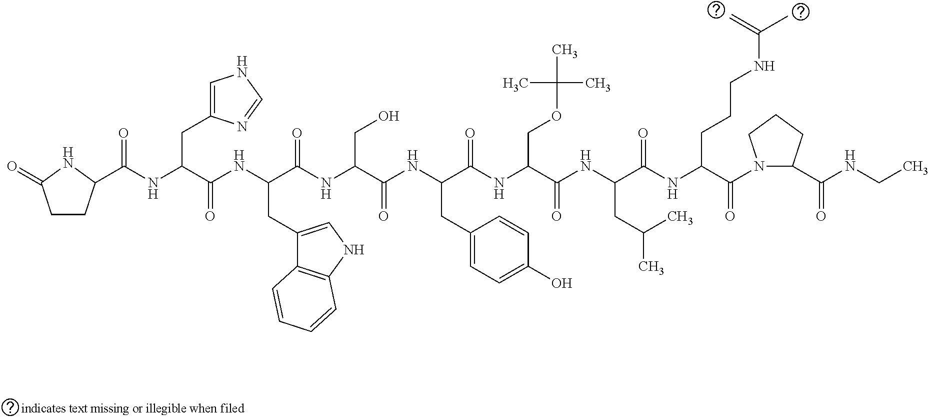

### (Buserelin);

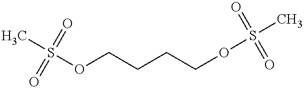

(Busulfan; 1,4-butanediol, dimethanesulfonate; sold as Busulfex® by ESP Pharma, Inc.; Edison, New Jersey);

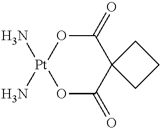

(Carboplatin; sold as Paraplatin® by Bristol-Myers Squibb; Princeton, N.J.);

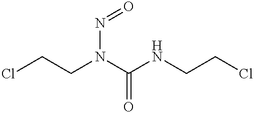

### (Carmustine);

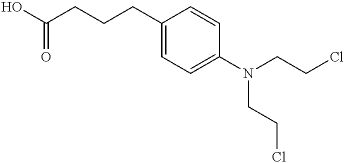

### (Chlorambucil);

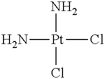

### (Cisplatin);

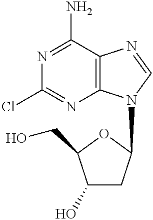

### (Cladribine);

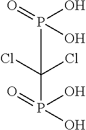

### (Clodronate);

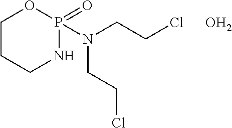

### (Cyclophosphamide);

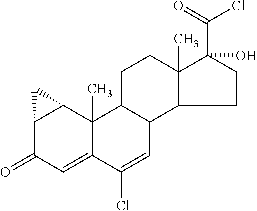

### (Cyproterone);

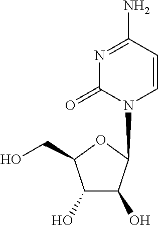

### (Cytarabine);

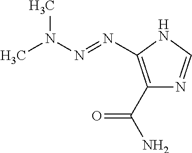

### (Dacarbazine);

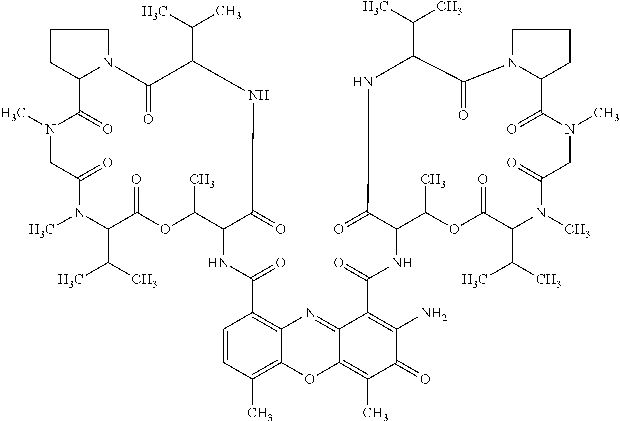

### (Dactinomycin);

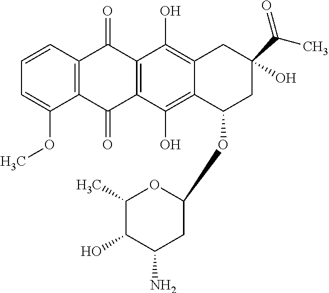

### (Daunorubicin);

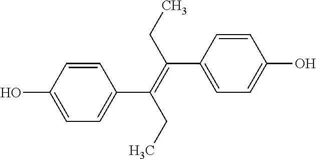

### (Diethylstilbestrol);

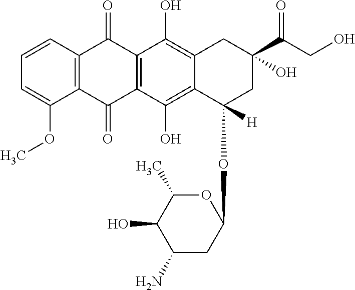

### (Epirubicin);

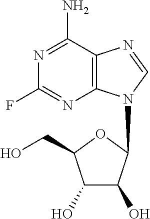

### (Fludarabine);

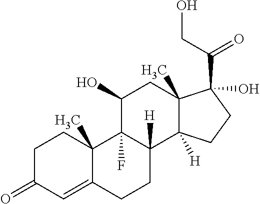

### (Fludrocortisone);

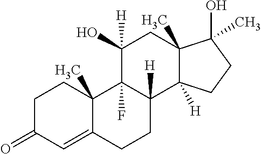

### (Fluoxymesterone);

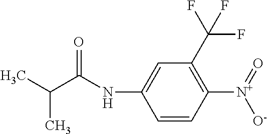

### (Flutamide);

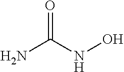

### (Hydroxyurea);

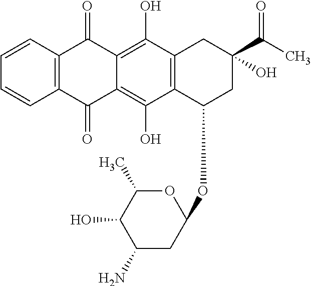

### (Idarubicin);

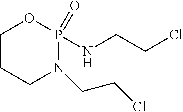

### (Ifosfamide);

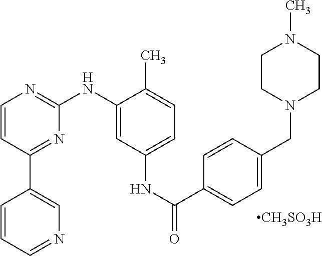

(Imatinib; sold as Gleevec® by Novartis Pharmaceuticals Corporation; East Hanover, N.J.);

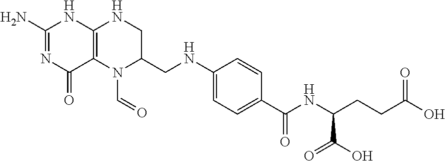

### (Leucovorin);

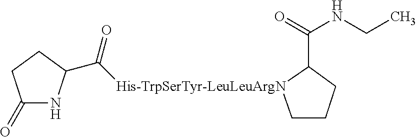

### (Leuprolide);

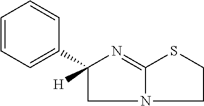

### (Levamisole);

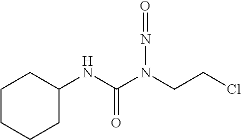

### (Lomustine);

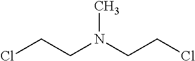

### (Mechlorethamine);

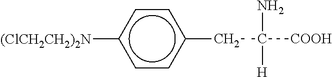

(Melphalan; sold as Alkeran® by Celgene Corporation; Warren, N.J.);

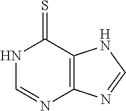

### (Mercaptopurine);

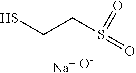

### (Mesna);

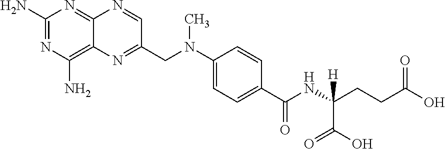

### (Methotrexate);

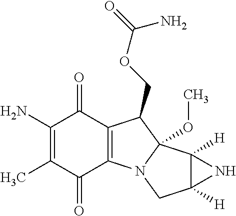

### (Mitomycin);

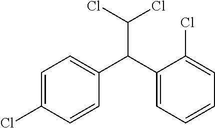

### (Mitotane);

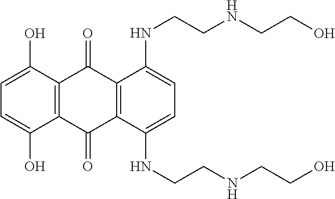

### (Mitoxantrone);

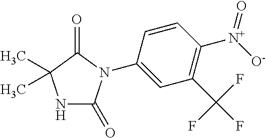

(Nilutamide); octreotide

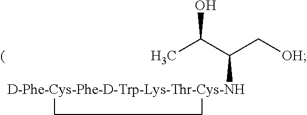

Katz et al., Clin Pharm. 8(4):255-73 (1989); sold as Sandostatin LAR® Depot; Novartis Pharm. Corp; E. Hanover, N.J.); edotreotide (yttrium-90 labeled or unlabeled); oxaliplatin

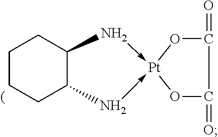

sold as Eloxatin™ by Sanofi-Synthelabo Inc.; New York, N.Y.);

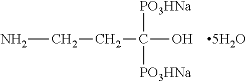

(Pamidronate; sold as Aredia® by Novartis Pharmaceuticals Corporation; East Hanover, N.J.);

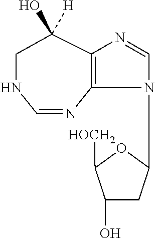

(Pentostatin; sold as Nipent® by Supergen; Dublin, Calif.);

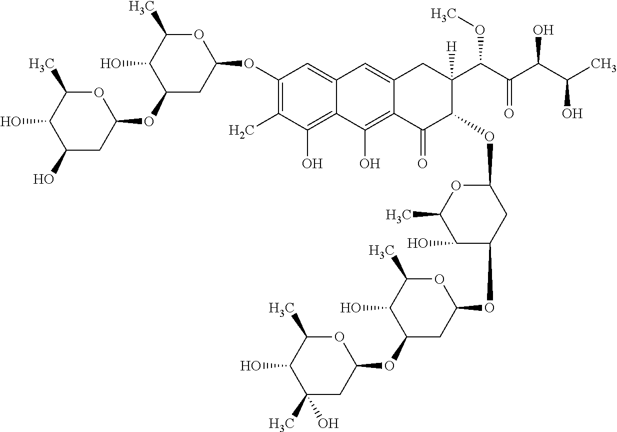

### (Plicamycin);

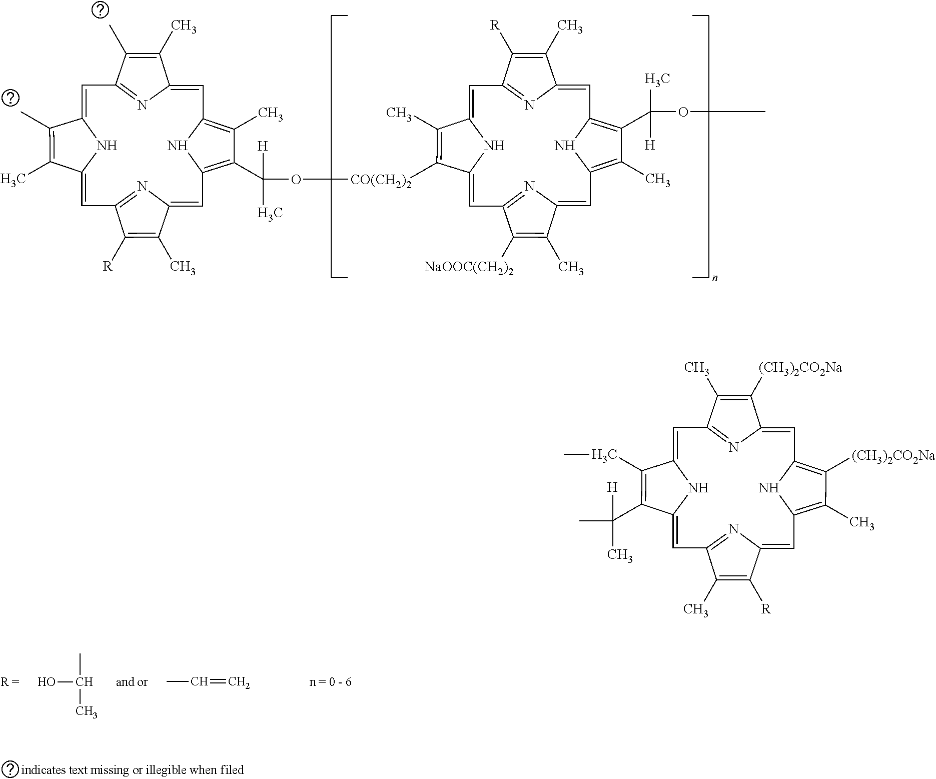

(Porfimer; sold as Photofrin® by Axcan Scandipharm Inc.; Birmingham, Ala.);

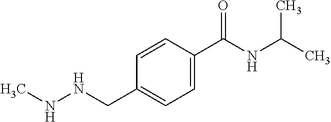

### (Procarbazine);

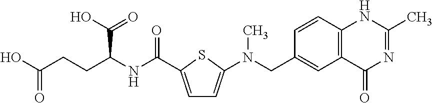

(Raltitrexed); Rituximab (sold as Rituxan® by Genentech, Inc.; South San Francisco, Calif.);

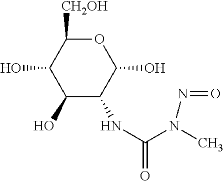

### (Streptozocin);

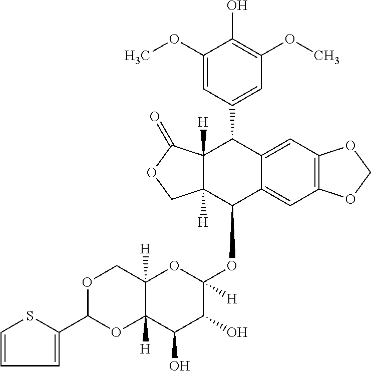

### (Teniposide);

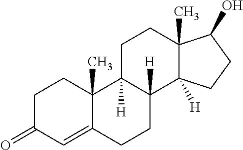

### (Testosterone);

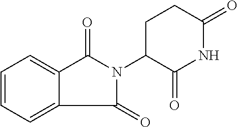

### (Thalidomide);

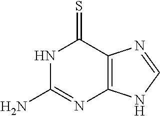

### (Thioguanine);

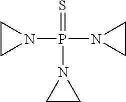

### (Thiotepa);

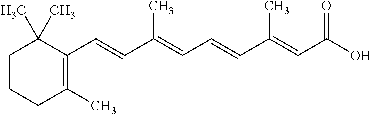

### (Tretinoin);

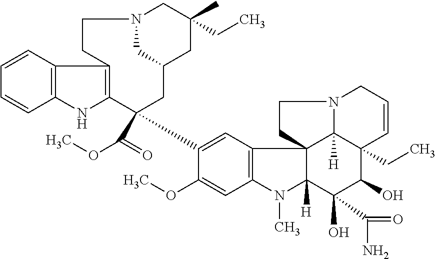

(Vindesine) or 13-cis-retinoic acid

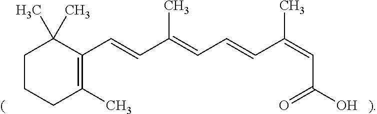

In an embodiment of the invention, an antibody or antigen-binding fragment thereof of the invention is provided in association with one or more of any of: phenylalanine mustard, uracil mustard, estramustine, altretamine, floxuridine, 5-deooxyuridine, cytosine arabinoside, 6-mercaptopurine, deoxycoformycin, calcitriol, valrubicin, mithramycin, vinblastine, vinorelbine, topotecan, razoxin, marimastat, COL-3, neovastat, BMS-275291, squalamine, endostatin, SU5416, SU6668, EMD121974, interleukin-12, IM862, angiostatin, vitaxin, droloxifene, idoxyfene, spironolactone, finasteride, cimitidine, trastuzumab, denileukin, diftitox, gefitinib, bortezimib, paclitaxel, docetaxel, epithilone B, BMS-247550 (see e.g., Lee et al., Clin. Cancer Res. 7:1429-1437 (2001)), BMS-310705, droloxifene (3-hydroxytamoxifen), 4-hydroxytamoxifen, pipendoxifene, ERA-923, arzoxifene, fulvestrant, acolbifene, lasofoxifene (CP-336156), idoxifene, TSE-424, HMR-3339, ZK186619, topotecan, PTK787/ZK 222584 (Thomas et al., Semin Oncol. 30(3 Suppl 6):32-8 (2003)), the humanized anti-VEGF antibody Bevacizumab, VX-745 (Haddad, Curr Opin. Investig. Drugs 2(8):1070-6 (2001)), PD 184352 (Sebolt-Leopold, et al. Nature Med. 5: 810-816 (1999)), any mTOR inhibitor, rapamycin

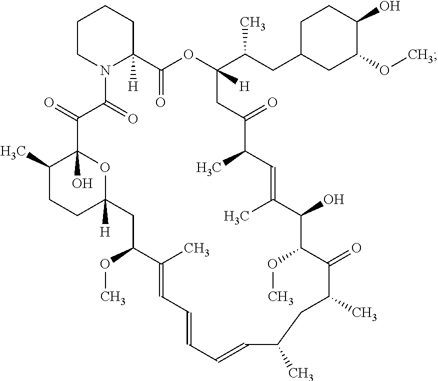

sirolimus), 40-O-(2-hydroxyethyl)-rapamycin, CCl-779

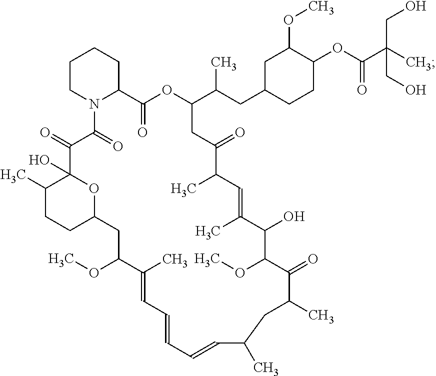

temsirolimus; Sehgal et al., Med. Res. Rev., 14:1-22 (1994); Elit, Curr. Opin. Investig. Drugs 3(8):1249-53 (2002)), AP-23573

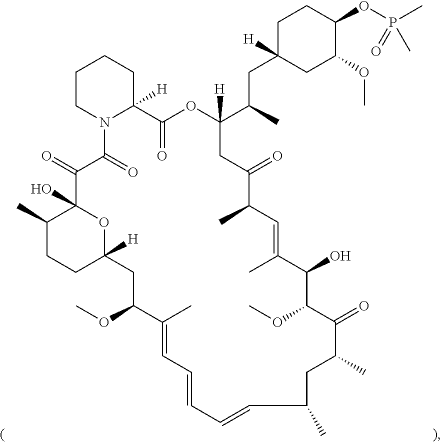

### RAD001

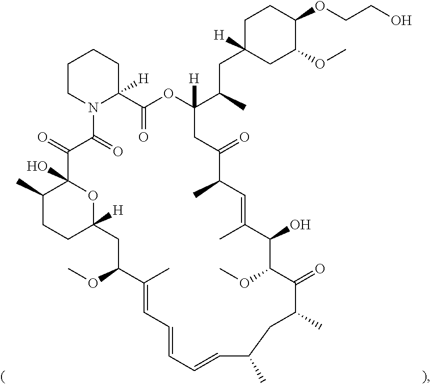

### ABT-578

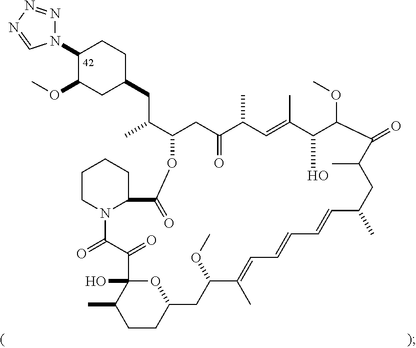

### BC-210

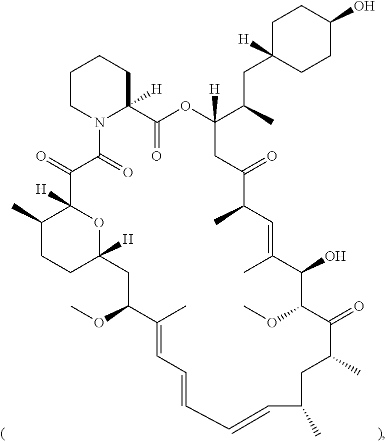

LY294002, LY292223, LY292696, LY293684, LY293646 (Vlahos et al., J. Biol. Chem. 269(7): 5241-5248 (1994)), wortmannin, BAY-43-9006, (Wilhelm et al., Curr. Pharm. Des. 8:2255-2257 (2002)), ZM336372, L-779,450, any Raf inhibitor disclosed in Lowinger et al., Curr. Pharm Des. 8:2269-2278 (2002); flavopiridol (L86-8275/HMR 1275; Senderowicz, Oncogene 19(56): 6600-6606 (2000)) or UCN-01 (7-hydroxy staurosporine; Senderowicz, Oncogene 19(56): 6600-6606 (2000)).

In an embodiment of the invention, an antibody or antigen-binding fragment thereof of the invention is provided in association with one or more of any of the compounds set forth in U.S. Pat. No. 5,656,655, which discloses styryl substituted heteroaryl EGFR inhibitors; in U.S. Pat. No. 5,646,153 which discloses bis mono and/or bicyclic aryl heteroaryl carbocyclic and heterocarbocyclic EGFR and PDGFR inhibitors; in U.S. Pat. No. 5,679,683 which discloses tricyclic pyrimidine compounds that inhibit the EGFR; in U.S. Pat. No. 5,616,582 which discloses quinazoline derivatives that have receptor tyrosine kinase inhibitory activity; in Fry et al., Science 265 1093-1095 (1994) which discloses a compound having a structure that inhibits EGFR (see FIG. 1 of Fry et al.); in U.S. Pat. No. 5,196,446 which discloses heteroarylethenediyl or heteroarylethenediylaryl compounds that inhibit EGFR; in Panek, et al., Journal of Pharmacology and Experimental Therapeutics 283: 1433-1444 (1997) which disclose a compound identified as PD166285 that inhibits the EGFR, PDGFR, and FGFR families of receptors-PD166285 is identified as 6-(2,6-dichlorophenyl)-2-(4-(2-diethylaminoethoxy)phenylamino)-8-methyl-8H-pyrido(2,3-d)pyrimidin-7-one.

In an embodiment of the invention, an antibody or antigen-binding fragment thereof of the invention is provided in association with one or more of any of: pegylated or unpegylated interferon alfa-2a, pegylated or unpegylated interferon alfa-2b, pegylated or unpegylated interferon alfa-2c, pegylated or unpegylated interferon alfa n-1, pegylated or unpegylated interferon alfa n-3 and pegylated, unpegylated consensus interferon or albumin-interferon-alpha.

The term “interferon alpha” as used herein means the family of highly homologous species-specific proteins that inhibit cellular proliferation and modulate immune response. Typical suitable interferon-alphas include, but are not limited to, recombinant interferon alpha-2b, recombinant interferon alpha-2a, recombinant interferon alpha-2c, alpha 2 interferon, interferon alpha-n1 (INS), a purified blend of natural alpha interferons, a consensus alpha interferon such as those described in U.S. Pat. Nos. 4,897,471 and 4,695,623 (especially Examples 7, 8 or 9 thereof), or interferon alpha-n3, a mixture of natural alpha interferons. Interferon alfa-2a is sold as ROFERON-A® by Hoffmann-La Roche (Nutley, N.J.).

Interferon alfa-2b is sold as INTRON-A® by Schering Corporation (Kenilworth, N.J.). The manufacture of interferon alpha 2b is described, for example, in U.S. Pat. No. 4,530,901.

Interferon alfa-n3 is a mixture of natural interferons sold as ALFERON N INJECTION® by Hemispherx Biopharma, Inc. (Philadelphia, Pa.).

Interferon alfa-n1 (INS) is a mixture of natural interferons sold as WELLFERON® by Glaxo-Smith-Kline (Research Triangle Park, N.C.).

Consensus interferon is sold as INFERGEN® by Intermune, Inc. (Brisbane, Calif.).

Interferon alfa-2c is sold as BEROFOR® by Boehringer Ingelheim Pharmaceutical, Inc. (Ridgefield, Conn.).

A purified blend of natural interferons is sold as SUMIFERON® by Sumitomo; Tokyo, Japan.

The term “pegylated interferon alpha” as used herein means polyethylene glycol modified conjugates of interferon alpha, preferably interferon alpha-2a and alpha-2b. The preferred polyethylene-glycol-interferon alpha-2b conjugate is PEG 12000-interferon alpha-2b. The phrases “12,000 molecular weight polyethylene glycol conjugated interferon alpha” and “PEG 12000-IFN alpha” as used herein include conjugates such as are prepared according to the methods of International Application No. WO 95/13090 and EP1039922 and containing urethane linkages between the interferon alpha-2a or -2b amino groups and polyethylene glycol having an average molecular weight of 12000. The pegylated inteferon alpha, PEG 12000-IFN-alpha-2b is available from Schering-Plough Research Institute, Kenilworth, N.J.

The preferred PEG 12000-interferon alpha-2b can be prepared by attaching a PEG polymer to the histidine residue in the interferon alpha-2b molecule. A single PEG 12000 molecule can be conjugated to free amino groups on an IFN alpha-2b molecule via a urethane linkage. This conjugate is characterized by the molecular weight of PEG 12000 attached. The PEG 12000-IFN alpha-2b conjugate can be formulated as a lyophilized powder for injection.

Pegylated interferon alfa-2b is sold as PEG-INTRON® by Schering Corporation (Kenilworth, N.J.).

Pegylated interferon-alfa-2a is sold as PEGASYS® by Hoffmann-La Roche (Nutley, N.J.).

Other interferon alpha conjugates can be prepared by coupling an interferon alpha to a water-soluble polymer. A non-limiting list of such polymers includes other polyalkylene oxide homopolymers such as polypropylene glycols, polyoxyethylenated polyols, copolymers thereof and block copolymers thereof. As an alternative to polyalkylene oxide-based polymers, effectively non-antigenic materials such as dextran, polyvinylpyrrolidones, polyacrylamides, polyvinyl alcohols, carbohydrate-based polymers and the like can be used. Such interferon alpha-polymer conjugates are described, for example, in U.S. Pat. No. 4,766,106, U.S. Pat. No. 4,917,888, European Patent Application No. 0 236 987 or 0 593 868 or International Publication No. WO 95/13090. The preferred PEG12000-IFN alfa 2b can be prepared by attaching in a PEG polymer to a histidine residue in the interferon alfa-2b molecule.

Pharmaceutical compositions of pegylated interferon alpha suitable for parenteral administration can be formulated with a suitable buffer, e.g., Tris-HCl, acetate or phosphate such as dibasic sodium phosphate/monobasic sodium phosphate buffer, and pharmaceutically acceptable excipients (e.g., sucrose), carriers (e.g. human plasma albumin), toxicity agents (e.g., NaCl), preservatives (e.g., thimerosol, cresol or benzyl alcohol), and surfactants (e.g., tween or polysorbates) in sterile water for injection. The pegylated interferon alpha can be stored as lyophilized powder under refrigeration at 2°-8° C. The reconstituted aqueous solutions are stable when stored between 2° and 8° C. and used within 24 hours of reconstitution. See for example U.S. Pat. Nos. 4,492,537; 5,762,923 and 5,766,582. The reconstituted aqueous solutions may also be stored in prefilled, multi-dose syringes such as those useful for delivery of drugs such as insulin. Typical, suitable syringes include systems comprising a prefilled vial attached to a pen-type syringe such as the NOVOLET®

Novo Pen available from Novo Nordisk or the REDIPEN®, available from Schering Corporation, Kenilworth, N.J. Other syringe systems include a pen-type syringe comprising a glass cartridge containing a diluent and lyophilized pegylated interferon alpha powder in a separate compartment.

The scope of the present invention also includes compositions comprising an antibody or antigen-binding fragment thereof of the invention in association with one or more other anti-cancer chemotherapeutic agents (e.g., as described herein) in association with one or more antiemetics including, but not limited to, casopitant (GlaxoSmithKline), Netupitant (MGI-Helsinn) and other NK-1 receptor antagonists, palonosetron (sold as Aloxi by MGI Pharma), aprepitant (sold as Emend by Merck and Co.; Rahway, N.J.), diphenhydramine (sold as Benadryl® by Pfizer; New York, N.Y.), hydroxyzine (sold as Atarax® by Pfizer; New York, N.Y.), metoclopramide (sold as Reglan® by AH Robins Co; Richmond, Va.), lorazepam (sold as Ativan® by Wyeth; Madison, N.J.), alprazolam (sold as Xanax® by Pfizer; New York, N.Y.), haloperidol (sold as Haldol® by Ortho-McNeil; Raritan, N.J.), droperidol (Inapsine®), dronabinol (sold as Marinol® by Solvay Pharmaceuticals, Inc.; Marietta, Ga.), dexamethasone (sold as Decadron® by Merck and Co.; Rahway, N.J.), methylprednisolone (sold as Medrol® by Pfizer; New York, N.Y.), prochlorperazine (sold as Compazine® by Glaxosmithkline; Research Triangle Park, N.C.), granisetron (sold as Kytril® by Hoffmann-La Roche Inc.; Nutley, N.J.), ondansetron (sold as Zofran® by Glaxosmithkline; Research Triangle Park, N.C.), dolasetron (sold as Anzemet® by Sanofi-Aventis; New York, N.Y.), tropisetron (sold as Navoban® by Novartis; East Hanover, N.J.).

Compositions comprising an antiemetic are useful for preventing or treating nausea; a common side effect of anti-cancer chemotherapy. Accordingly, the present invention also includes methods for treating or preventing cancer in a subject by administering an antibody or antigen-binding fragment thereof of the invention optionally in association with one or more other chemotherapeutic agents (e.g., as described herein) and/or optionally in association with one or more antiemetics.

Other side effects of cancer treatment include red and white blood cell deficiency. Accordingly, the present invention includes compositions comprising an anti-IGF1R antibody or antigen-binding fragment thereof optionally in association with an agent which treats or prevents such a deficiency, such as, e.g., pegfilgrastim, erythropoietin, epoetin alfa or darbepoetin alfa.

The present invention further comprises a method for treating or preventing any stage or type of any medical condition set forth herein by administering an antibody or antigen-binding fragment thereof of the invention in association with a therapeutic procedure such as surgical tumorectomy or anti-cancer radiation treatment; optionally in association with a further chemotherapeutic agent and/or antiemetic, for example, as set forth above.

The term “in association with” indicates that the components of a composition of the invention (e.g., anti-IGF1R antibody or antigen-binding fragment thereof along with docetaxel) can be formulated into a single composition for simultaneous delivery or formulated separately into two or more compositions (e.g., a kit). Furthermore, each component can be administered to a subject at a different time than when the other component is administered; for example, each administration may be given non-simultaneously (e.g., separately or sequentially) at several intervals over a given period of time. Moreover, the separate components may be administered to a subject by the same or by a different route (e.g., wherein an anti-IGF1R antibody is administered parenterally and gefitinib is administered orally).

### Therapeutic Methods, Dosage and Administration

Methods of the present invention include provision and/or administration of an IGF1R antibody or antigen-binding fragment thereof, optionally, in a pharmaceutical formulation as set forth herein, optionally in association with a further therapeutic agent, including anti-cancer agents and anti-emetics and other treatments for complications arising out of chemotherapy or radiation therapy, or a pharmaceutical composition thereof to treat or prevent cancer or any medical disorder mediated by elevated expression and/or activity of IGF1R and/or elevated expression (e.g., blood levels) of IGF-1 and/or IGF-2. Typically, the administration and dosage of such further agents is, when possible, done according to the schedule listed in the product information sheet of the approved agents, in the Physicians' Desk Reference 2003 (Physicians' Desk Reference, 57th Ed); Medical Economics Company; ISBN: 1563634457; 57th edition (November 2002), as well as therapeutic protocols well known in the art.

In an embodiment, an antibody or antigen-binding fragment thereof of the invention is administered to a subject parenterally, for example, by intravenous, intrathecal, subcutaneous, intramuscular, intratumoral or intraarterial injection. In an embodiment, the antibody or antigen-binding fragment thereof is administered orally or by inhalation.

Medical conditions treatable or preventable by administering an anti-IGF1R antibody or antigen-binding fragment thereof or a combination thereof of the present invention include, but are not limited to osteosarcoma, rhabdomyosarcoma, neuroblastoma, any pediatric cancer, kidney cancer, leukemia, renal transitional cell cancer, Werner-Morrison syndrome, acromegaly, bladder cancer, Wilm's cancer, ovarian cancer, pancreatic cancer, benign prostatic hyperplasia, breast cancer, prostate cancer, bone cancer, lung cancer, gastric cancer, colorectal cancer, cervical cancer, synovial sarcoma, diarrhea associated with metastatic carcinoid, vasoactive intestinal peptide secreting tumors, gigantism, psoriasis, atherosclerosis, smooth muscle restenosis of blood vessels and inappropriate microvascular proliferation, head and neck cancer, squamous cell carcinoma, multiple myeloma, solitary plasmacytoma, renal cell cancer, retinoblastoma, germ cell tumors, hepatoblastoma, hepatocellular carcinoma, melanoma, rhabdoid tumor of the kidney, Ewing Sarcoma, chondrosarcoma, any haemotological malignancy (e.g., chronic lymphoblastic leukemia, chronic myelomonocytic leukemia, acute lymphoblastic leukemia, acute lymphocytic leukemia, acute myelogenous leukemia, acute myeloblastic leukemia, chronic myeloblastic leukemia, Hodgekin's disease, non-Hodgekin's lymphoma, chronic lymphocytic leukemia, chronic myelogenous leukemia, myelodysplastic syndrome, hairy cell leukemia, mast cell leukemia, mast cell neoplasm, follicular lymphoma, diffuse large cell lymphoma, mantle cell lymphoma, Burkitt Lymphoma, mycosis fungoides, seary syndrome, cutaneous T-cell lymphoma, chronic myeloproliferative disorders), and cental nervous system tumors (e.g., brain cancer, glioblastoma, non-glioblastoma brain cancer, meningioma, pituitary adenoma, vestibular schwannoma, a primitive neuroectodermal tumor, medulloblastoma, astrocytoma, anaplastic astrocytoma, oligodendroglioma, ependymoma and choroid plexus papilloma), myeloproliferative disorders (e.g., polycythemia vera, thrombocythemia, idiopathic myelfibrosis), soft tissue sarcoma, thyroid cancer, endometrial cancer, carcinoid cancer, germ cell tumors, liver cancer, gigantism, psoriasis, atherosclerosis, smooth muscle restenosis of blood vessels, inappropriate microvascular proliferation, acromegaly, gigantism, psoriasis, atherosclerosis, smooth muscle restenosis of blood vessels or inappropriate microvascular proliferation, Grave's disease, multiple sclerosis, systemic lupus erythematosus, Hashimoto's Thyroiditis, Myasthenia Gravis, auto-immune thyroiditis and Bechet's disease.

The anti-IGF1R antibodies and antigen-binding fragments thereof and compositions thereof are, in an embodiment of the invention, administered at a therapeutically effective dosage. The term “therapeutically effective amount” or “therapeutically effective dosage” means that amount or dosage of an antibody or antigen-binding fragment thereof of the invention or composition thereof that will elicit a biological or medical response of a tissue, system, patient, subject or host that is being sought by the administrator (such as a researcher, doctor or veterinarian) which includes any measurable alleviation of the signs, symptoms and/or clinical indicia of a medical disorder, such as cancer (e.g., tumor growth and/or metastasis) including the prevention, slowing or halting of progression of the medical disorder to any degree whatsoever. For example, in one embodiment of the invention, a “therapeutically effective dosage” of any anti-IGF1R antibody or antigen-binding fragment thereof of the present invention (e.g., an anti-IGF1R antibody comprising mature 2C6 or 9H2 light chain and/or mature 2C6 or 9H2 heavy chain) is between about 0.3 and 20 mg/kg of body weight (e.g., about 0.3 mg/kg of body weight, about 0.6 mg/kg of body weight, about 0.9 mg/kg of body weight, about 1 mg/kg of body weight, about 2 mg/kg of body weight, about 3 mg/kg of body weight, about 4 mg/kg of body weight, about 5 mg/kg of body weight, about 6 mg/kg of body weight, about 7 mg/kg of body weight, about 8 mg/kg of body weight, about 9 mg/kg of body weight, about 10 mg/kg of body weight, about 11 mg/kg of body weight, about 12 mg/kg of body weight, about 13 mg/kg of body weight, about 14 mg/kg of body weight, about 15 mg/kg of body weight, about 16 mg/kg of body weight, about 17 mg/kg of body weight, about 18 mg/kg of body weight, about 19 mg/kg of body weight, about 20 mg/kg of body weight), about once per week to about once every 3 weeks (e.g., about once every 1 week or once every 2 weeks or once every 3 weeks). As mentioned above, the therapeutically effective dosage of a further therapeutic agent is, when possible, as set forth in the Physicians' Desk Reference.

Dosage regimens may be adjusted to provide the optimum desired response (e.g., a therapeutic response). For example, a single dose may be administered or several divided doses may be administered over time or the dose may be proportionally reduced or increased as indicated by the exigencies or the particular circumstances or requirements of the therapeutic situation. For example, dosage may be determined or adjusted, by a practitioner of ordinary skill in the art (e.g., physician or veterinarian) according to the patient's age, weight, height, past medical history, present medications and the potential for cross-reaction, allergies, sensitivities and adverse side-effects. For example, the physician or veterinarian could start doses of the antibody or antigen-binding fragment of the invention or composition thereof at levels lower than that required in order to achieve the desired therapeutic effect and gradually increase the dosage until the desired effect is achieved. The effectiveness of a given dose or treatment regimen of an antibody or combination of the invention can be determined, for example, by determining whether a tumor being treated in the subject shrinks or ceases to grow. The size and progress of a tumor can be easily determined, for example, by X-ray, magnetic resonance imaging (MRI) or visually in a surgical procedure. In general, tumor size and proliferation can be measured by use of a thymidine PET scan (see e.g., Wells et al., Clin. Oncol. 8: 7-14 (1996)). Generally, the thymidine PET scan includes the injection of a radioactive tracer, such as [2-¹¹C]-thymidine, followed by a PET scan of the patient's body (Vander Borght et al., Gastroenterology 101: 794-799, 1991; Vander Borght et al., J. Radiat. Appl. Instrum. Part A, 42: 103-104 (1991)). Other tracers that can be used include [¹⁸F]-FDG (18-fluorodeoxyglucose), [¹²⁴I]IUdR (5-[124I]iodo-2′-deoxyuridine), [⁷⁶Br]BrdUrd (Bromodeoxyuridine), [¹⁸F]FLT (3′-deoxy-3′ fluorothymidine) or [¹¹C]FMAU (2′-fluoro-5-methyl-1-β-D-arabinofuranosyluracil).

For example, neuroblastoma progress can be monitored, by a physician, by a variety of methods, and the dosing regimen can be altered accordingly. Methods by which to monitor neuroblastoma include, for example, CT scan (e.g., to monitor tumor size), MRI scan (e.g., to monitor tumor size), chest X-ray (e.g., to monitor tumor size), bone scan, bone marrow biopsy (e.g., to check for metastasis to the bone marrow), hormone tests (levels of hormones like epinephrine), complete blood test (CBC) (e.g., to test for anemia or other abnormality), testing for catecholamines (a neuroblastoma tumor marker) in the urine or blood, a 24 hour urine test for check for homovanillic acid (HMA) or vanillyl mandelic acid (VMA) levels (neuroblastoma markers) and an MIBG scan (scan for injected I¹²³-labeled metaiodobetaguanidine; e.g., to monitor adrenal tumors).

For example, rhabdomyosarcoma progress can be monitored, by the physician, by a variety of methods, and the dosing regimen can be altered accordingly. Methods by which to monitor rhabdomyosarcoma include, for example tumor biopsy, CT scan (e.g., to monitor tumor size), MRI scan (e.g., to monitor tumor size), CT scan of the chest (e.g., to monitor metastases), bone scan (e.g., to monitor metastases), bone marrow biopsy (e.g., to monitor metastases), spinal tap (e.g., to check for metastasis into the brain) and a thorough physical exam.

For example, osteosarcoma progress can be monitored, by the physician, by a variety of methods, and the dosing regimen can be altered accordingly. Methods by which to monitor osteosarcoma include, for example, X-ray of the affected area or of the chest (e.g., to check for spread to the lungs), CT scan of the affected area, blood tests (e.g., to measure alkaline phosphatase levels), CT scan of the chest to see if the cancer has spread to the lungs, open biopsy, or a bone scan to see if the cancer has spread to other bones.

For example, pancreatic cancer progress can be monitored, by the physician, by a variety of methods, and the dosing regimen can be altered accordingly. Methods by which to monitor pancreatic cancer include blood tests to check for tumor markers CA 19-9 and/or carcinoembryonic antigen (CEA), an upper GI series (e.g., a barium swallow), endoscopic ultrasonography; endoscopic retrograde cholangiopancreatography (an x-ray of the pancreatic duct and bile ducts); percutaneous transhepatic cholangiography (an x-ray of the bile duct), abdominal ultrasound imaging or abdominal CT scan.

For example, bladder cancer progress can be monitored, by the physician, by a variety of methods, and the dosing regimen can be altered accordingly. Methods by which to monitor bladder cancer include urinalysis to detect elevated levels of tumor markers (e.g., nuclear matrix protein (NMP22)) in the urine, urinalysis to detect microscopic hematuria, urine cytology to detect cancer cells by examining cells flushed from the bladder during urination, bladder cystoscopy, intravenous pyelogram (IVP), retrograde pyelography, chest X-ray to detect metastasis, computed tomography (CT), bone scan, MRI scan, PET scan or biopsy.

For example, breast cancer progress can be monitored, by the physician, by a variety of methods, and the dosing regimen can be altered accordingly. Methods by which to monitor breast cancer include mammography, aspiration or needle biopsy or palpation.

For example, lung cancer progress can be monitored, by the physician, by a variety of methods, and the dosing regimen can be altered accordingly. Methods by which to monitor lung cancer include chest X-ray, CT scan, low-dose helical CT scan (or spiral CT scan), MRI scan, PET scan, bone scan, sputum cytology, bronchoscopy, mediastinoscopy, biopsy (e.g., needle or surgical), thoracentesis or blood tests to detect PTH (parathyroid hormone), CEA (carcinogenic antigen) or CYFRA21-1 (cytokeratin fragment 19).

For example, prostate cancer progress can be monitored, by the physician, by a variety of methods, and the dosing regimen can be altered accordingly. Methods by which to monitor prostate cancer include digital rectal examination, transrectal ultrasound, blood tests taken to check the levels of prostate specific antigen (PSA) and prostatic acid phosphatase (PAP), biopsy, bone scan and CT scan.

For example, colorectal or colon cancer progress can be monitored, by the physician, by a variety of methods, and the dosing regimen can be altered accordingly.

Methods by which to monitor colorectal or colon cancer include CT scan, MRI scan, chest X-ray, PET scan, fecal occult blood tests (FOBTs), flexible proctosigmoidoscopy, total colonoscopy, and barium enema.

For example, cervical cancer progress can be monitored, by the physician, by a variety of methods, and the dosing regimen can be altered accordingly. Methods by which to monitor cervical cancer include PAP smear, pelvic exam, colposcopy, cone biopsy, endocervical curettage, X-ray, CT scan, cystoscopy and proctoscopy.

For example, gastric cancer progress can be monitored, by the physician, by a variety of methods, and the dosing regimen can be altered accordingly. Methods by which to monitor gastric cancer include esophagogastroduodenoscopy (EGD), double-contrast barium swallow, endoscopic biopsy, computed tomographic (CT) scanning, magnetic resonance imagine (MRI) or endoscopic ultrasonography (EUS).

For example, Wilm's cancer progress can be monitored, by the physician, by a variety of methods, and the dosing regimen can be altered accordingly. Methods by which to monitor Wilm's cancer include abdominal computer tomography scan (CT), abdominal ultrasound, blood and urine tests to evaluate kidney and liver function, chest X-ray to check for metastasis, magnetic resonance imaging (MRI), blood tests and urinalysis to assay kidney function and biopsy.

In an embodiment of the invention, any patient suffering from a cancer whose tumor cells express IGF1R is selected for treatment with an antibody or antigen-binding fragment thereof of the invention. In an embodiment of the invention, a patient whose tumor exhibits any of the following characteristics is selected for treatment with an antibody or antigen-binding fragment thereof of the invention: IRS-1 phosphorylation on tyrosine 896; (ii) IRS-1 phosphorylation on tyrosine 612; (iii) IRS-1 phosphorylation on any tyrosine; (iv) IGF-II; and/or (v) IGF1R phosphorylation on any tyrosine. Such characteristics can be identified in an tumor cell by any of several methods commonly known in the art (e.g., ELISA or western blot) (See e.g., US2006/140960).

Pharmaceutical compositions comprising an antibody or antigen-binding fragment thereof of the invention in association with a pharmaceutically acceptable carrier are also within the scope of the present invention (e.g., in a single composition or separately in a kit). The pharmaceutical compositions may be prepared by any methods well known in the art of pharmacy; see, e.g., Gilman, et al., (eds.) (1990), The Pharmacological Bases of Therapeutics, 8th Ed., Pergamon Press; A. Gennaro (ed.), Remington's Pharmaceutical Sciences, 18th Edition, (1990), Mack Publishing Co., Easton, Pa.; Avis, et al., (eds.) (1993) Pharmaceutical Dosage Forms: Parenteral Medications Dekker, New York; Lieberman, et al., (eds.) (1990) Pharmaceutical Dosage Forms: Tablets Dekker, New York; and Lieberman, et al., (eds.) (1990), Pharmaceutical Dosage Forms: Disperse Systems Dekker, New York. In an embodiment of the invention, the antibody or antigen-binding fragment thereof is administered to a subject as part of a pharmaceutical composition comprising sodium acetate (e.g., Trihydrate USP) at 2.30 mg/ml; glacial acetic acid (e.g., USP/Ph. Eur) at 0.18 mg/ml; sucrose (e.g., extra pure NF, Ph. Eur, BP) at 70.0 mg/ml; anti-IGF1R antibody or an antigen-binding fragment thereof at 20.0 mg/ml and water, for example, sterile water (e.g., for injection USP/Ph. Eur); at a pH of about 5.5 to about 6.0 (e.g., 5.5., 5.6, 5.7, 5.8, 5.9, 6.0). If a lyophilized powder thereof (also part of the present invention) is prepared, water is added to reconstitute the composition for use.

The term “subject” or “patient” includes any organism, such as a mammal (e.g., primate, dog, horse, rat, mouse, cat, rabbit) including a human. In an embodiment of the invention, a “subject” or “patient” is an adult human (e.g., 18 years or older) or a human child (e.g., under 18 years of age, for example, less than 1, 1, 2, 3, 4, 5, 6, 7, 8, 9 or 10 years of age).

A pharmaceutical composition containing an antibody or antigen-binding fragment thereof of the invention, which is optionally in association with a further chemotherapeutic agent, can be prepared using conventional pharmaceutically acceptable excipients and additives and conventional techniques. Such pharmaceutically acceptable excipients and additives include non-toxic compatible fillers, binders, disintegrants, buffers, preservatives, anti-oxidants, lubricants, flavorings, thickeners, coloring agents, emulsifiers and the like. All routes of administration are contemplated including, but not limited to, parenteral (e.g., subcutaneous, intravenous, intraperitoneal, intramuscular, topical, intra-peritoneal, inhalation, intra-cranial) and non-parenteral (e.g., oral, transdermal, intranasal, intraocular, sublingual, rectal and topical).

Injectables can be prepared in conventional forms, either as liquid solutions or suspensions, solid forms suitable for solution or suspension in liquid prior to injection, or as emulsions. The injectables, solutions and emulsions can also contain one or more excipients. Excipients include, for example, water, saline, dextrose, glycerol or ethanol. In addition, if desired, the pharmaceutical compositions to be administered may also contain minor amounts of non-toxic auxiliary substances such as wetting or emulsifying agents, pH buffering agents, stabilizers, solubility enhancers, and other such agents, such as for example, sodium acetate, sorbitan monolaurate, triethanolamine oleate and cyclodextrins.

In an embodiment of the invention, pharmaceutically acceptable carriers used in parenteral preparations include aqueous vehicles, nonaqueous vehicles, antimicrobial agents, isotonic agents, buffers, antioxidants, local anesthetics, suspending and dispersing agents, emulsifying agents, sequestering or chelating agents and other pharmaceutically acceptable substances.

Examples of aqueous vehicles include Sodium Chloride Injection, Ringers Injection, Isotonic Dextrose Injection, Sterile Water Injection, Dextrose and Lactated Ringers Injection. Nonaqueous parenteral vehicles include fixed oils of vegetable origin, cottonseed oil, corn oil, sesame oil and peanut oil. Antimicrobial agents in bacteriostatic or fungistatic concentrations may be added to parenteral preparations packaged in multiple-dose containers which include phenols or cresols, mercurials, benzyl alcohol, chlorobutanol, methyl and propyl p-hydroxybenzoic acid esters, thimerosal, benzalkonium chloride and benzethonium chloride. Isotonic agents include sodium chloride and dextrose. Buffers include phosphate and citrate. Antioxidants include sodium bisulfate. Local anesthetics include procaine hydrochloride. Suspending and dispersing agents include sodium carboxymethylcelluose, hydroxypropyl methylcellulose and polyvinylpyrrolidone. Emulsifying agents include Polysorbate 80 (TWEEN-80). A sequestering or chelating agent of metal ions includes EDTA (ethylenediaminetetraacetic acid) or EGTA (ethylene glycol tetraacetic acid). Pharmaceutical carriers may also include ethyl alcohol, polyethylene glycol and propylene glycol for water miscible vehicles; and sodium hydroxide, hydrochloric acid, citric acid or lactic acid for pH adjustment.

In an embodiment of the invention, preparations for parenteral administration can include sterile solutions ready for injection, sterile dry soluble products, such as lyophilized powders, ready to be combined with a solvent just prior to use, including hypodermic tablets, sterile suspensions ready for injection, sterile dry insoluble products ready to be combined with a vehicle just prior to use and sterile emulsions. The solutions may be either aqueous or nonaqueous.

The concentration of the antibody or antigen-binding fragment thereof of the invention, which is optionally in association with a further chemotherapeutic agent, can be adjusted so that an injection provides an effective amount to produce the desired pharmacological effect. As discussed below, the exact dose depends on the age, weight and condition of the patient or animal as is known in the art.

In an embodiment of the invention, unit-dose parenteral preparations are packaged in an ampoule, a vial or a syringe with a needle. All preparations for parenteral administration must be sterile, as is known and practiced in the art.

In an embodiment of the invention, a sterile, lyophilized powder is prepared by dissolving the antibody or antigen-binding fragment thereof, which is optionally in association with a further chemotherapeutic agent, or a pharmaceutically acceptable derivative thereof, in a suitable solvent. The solvent may contain an excipient which improves the stability or other pharmacological components of the powder or reconstituted solution, prepared from the powder. Excipients that may be used include, but are not limited to, dextrose, sorbital, fructose, corn syrup, xylitol, glycerin, glucose, sucrose or other suitable agent. The solvent may also contain a buffer, such as citrate, sodium or potassium phosphate or other such buffer known to those of skill in the art at, in one embodiment, about neutral pH. Subsequent sterile filtration of the solution followed by lyophilization under standard conditions known to those of skill in the art provides a desirable formulation. In one embodiment, the resulting solution will be apportioned into vials for lyophilization. Each vial can contain a single dosage or multiple dosages of the anti-IGF1R antibody or antigen-binding fragment thereof or composition thereof. Overfilling vials with a small amount above that needed for a dose or set of doses (e.g., about 10%) is acceptable so as to facilitate accurate sample withdrawal and accurate dosing. The lyophilized powder can be stored under appropriate conditions, such as at about 4° C. to room temperature.

Reconstitution of a lyophilized powder with water for injection provides a formulation for use in parenteral administration. In an embodiment of the invention, for reconstitution, the lyophilized powder is added to sterile water or other liquid suitable carrier. The precise amount depends upon the selected therapy being given. Such amount can be empirically determined.

Administration by inhalation can be provided by using, e.g., an aerosol containing sorbitan trioleate or oleic acid, for example, together with trichlorofluoromethane, dichlorofluoromethane, dichlorotetrafluoroethane or any other biologically compatible propellant gas; it is also possible to use a system containing an IGF1R inhibitor, which is optionally in association with a further chemotherapeutic agent, by itself or associated with an excipient, in powder form.

In an embodiment of the invention, the antibody or antigen-binding fragment thereof, which is optionally in association with a further chemotherapeutic agent, is formulated into a solid dosage form for oral administration, in one embodiment, into a capsule or tablet. Tablets, pills, capsules, troches and the like can contain one or more of the following ingredients, or compounds of a similar nature: a binder; a lubricant; a diluent; a glidant; a disintegrating agent; a coloring agent; a sweetening agent; a flavoring agent; a wetting agent; an emetic coating; and a film coating. Examples of binders include microcrystalline cellulose, gum tragacanth, glucose solution, acacia mucilage, gelatin solution, molasses, polyinylpyrrolidine, povidone, crospovidones, sucrose and starch paste. Lubricants include talc, starch, magnesium or calcium stearate, lycopodium and stearic acid. Diluents include, for example, lactose, sucrose, starch, kaolin, salt, mannitol and dicalcium phosphate. Glidants include, but are not limited to, colloidal silicon dioxide. Disintegrating agents include crosscarmellose sodium, sodium starch glycolate, alginic acid, corn starch, potato starch, bentonite, methylcellulose, agar and carboxymethylcellulose. Coloring agents include, for example, any of the approved certified water soluble FD and C dyes, mixtures thereof; and water insoluble FD and C dyes suspended on alumina hydrate. Sweetening agents include sucrose, lactose, mannitol and artificial sweetening agents such as saccharin, and any number of spray dried flavors. Flavoring agents include natural flavors extracted from plants such as fruits and synthetic blends of compounds which produce a pleasant sensation, such as, but not limited to peppermint and methyl salicylate. Wetting agents include propylene glycol monostearate, sorbitan monooleate, diethylene glycol monolaurate and polyoxyethylene laural ether. Emetic-coatings include fatty acids, fats, waxes, shellac, ammoniated shellac and cellulose acetate phthalates. Film coatings include hydroxyethylcellulose, sodium carboxymethylcellulose, polyethylene glycol 4000 and cellulose acetate phthalate.

Implantation of a slow-release or sustained-release system, such that a constant level of dosage is maintained, is also contemplated herein. Briefly, an active agent (e.g., anti-IGF1R, which is optionally in association with a further chemotherapeutic agent) is dispersed in a solid inner matrix, e.g., polymethylmethacrylate, polybutylmethacrylate, plasticized or unplasticized polyvinylchloride, plasticized nylon, plasticized polyethyleneterephthalate, natural rubber, polyisoprene, polyisobutylene, polybutadiene, polyethylene, ethylene-vinylacetate copolymers, silicone rubbers, polydimethylsiloxanes, silicone carbonate copolymers, hydrophilic polymers such as hydrogels of esters of acrylic and methacrylic acid, collagen, cross-linked polyvinylalcohol and cross-linked partially hydrolyzed polyvinyl acetate, that is surrounded by an outer polymeric membrane, e.g., polyethylene, polypropylene, ethylene/propylene copolymers, ethylene/ethyl acrylate copolymers, ethylene/vinylacetate copolymers, silicone rubbers, polydimethyl siloxanes, neoprene rubber, chlorinated polyethylene, polyvinylchloride, vinylchloride copolymers with vinyl acetate, vinylidene chloride, ethylene and propylene, ionomer polyethylene terephthalate, butyl rubber epichlorohydrin rubbers, ethylene/vinyl alcohol copolymer, ethylene/vinyl acetate/vinyl alcohol terpolymer, and ethylene/vinyloxyethanol copolymer, that is insoluble in body fluids. The compound diffuses through the outer polymeric membrane in a release rate controlling step. The percentage of active compound contained in such parenteral compositions is highly dependent on the specific nature thereof, as well as the activity of the antibody or antigen-binding fragment, which is optionally in association with a further chemotherapeutic agent, and the needs of the subject.

Any of the agents set forth herein can be formulated into a sustained release formulation including liposomal formulations such as unilamellar vesicular (ULV) and multilamellar vesicular (MLV) liposomes and DepoFoam™ particles (Kim et al., Biochim. Biophys. Acta (1983) 728(3):339-348; Kim, Methods Neurosci. (1994) 21: 118-131; Kim et al., Anesthesiology (1996) 85(2): 331-338; Katre et al., J. Pharm. Sci. (1998) 87(11): 1341-1346). A feature of the DepoFoam system is that, inside each DepoFoam particle, discontinuous internal aqueous chambers, bounded by a continuous, non-concentric network of lipid membranes render a higher aqueous volume-to-lipid ratio and much larger particle diameters compared with MLV.

### Experimental and Diagnostic Uses

The antibodies and fragments of the invention may be used as affinity purification agents. In this process, the antibodies or fragments are immobilized on a solid phase such a Sephadex resin or filter paper, using methods well known in the art. The immobilized antibody or fragment is contacted with a sample containing the IGF1R protein (or fragment thereof) to be purified, and thereafter the support is washed with a suitable solvent that will remove substantially all the material in the sample except the IGF1R protein, which is bound to the immobilized antibody or fragment. Finally, the support is washed with a solvent which elutes the bound IGF1R from the column (e.g., protein A). Such immobilized antibodies form part of the present invention.

The present invention also provides antigens for generating secondary antibodies which are useful for example for performing Western blots and other immunoassays discussed herein. Specifically, the present invention includes polypeptides comprising the variable regions and/or CDR sequences of 2C6 or 9H2 which may be used to generate an anti-2C6 or anti-9H2 secondary antibody. Detectably labeled anti-2C6 or anti-9H2 secondary antibodies are within the scope of the present invention.

Anti-IGF1R antibodies or fragments thereof may also be useful in diagnostic assays for IGF1R protein, e.g., detecting its expression in specific cells, tissues, or serum. Such diagnostic methods may be useful in cancer diagnosis.

For example, embodiments of the invention include ELISA assays (enzyme-linked immunosorbent assay) incorporating the use of an anti-IGF1R antibody or fragment thereof of the invention. For example, in an embodiment of the invention, such a method comprises the following steps:

(a) coat a substrate (e.g., surface of a microtiter plate well, e.g., a plastic plate) with anti-IGF1R antibody or antigen-binding fragment thereof;

(b) apply a sample to be tested for the presence of IGF1R to the substrate;

(c) wash the plate, so that unbound material in the sample is removed;

(d) apply detectably labeled antibodies (e.g., enzyme-linked antibodies) which are also specific to the IGF1R antigen;

(e) wash the substrate, so that the unbound, labeled antibodies are removed;

(f) if the labeled antibodies are enzyme linked, apply a chemical which is converted by the enzyme into a fluorescent signal;

(g) detect the presence of the labeled antibody.

In an embodiment of the invention, the labeled antibody is labeled with peroxidase which react with ABTS (e.g., 2,2′-azino-bis(3-ethylbenzthiazoline-6-sulphonic acid)) or 3,3′,5,5′-Tetramethylbenzidine to produce a color change which is detectable.

Alternatively, the labeled antibody is labeled with a detectable radioisotope (e.g., ³H) which can be detected by scintillation counter in the presence of a scintillant.

An anti-IGF1R antibody of the invention may be used in a Western blot or immuno protein blot procedure. Such a procedure forms part of the present invention and includes e.g.,:

(1) contacting a membrane or other solid substrate to be tested for the presence of bound IGF1R or a fragment thereof with an anti-IGF1R antibody or antigen-binding fragment thereof of the invention. Such a membrane may take the form of a nitrocellulose or vinly-based (e.g., polyvinylidene fluoride (PVDF)) membrane to which proteins to be tested for the presence of IGF1R in a non-denaturing PAGE (polyacrylamide gel electrophoresis) gel or SDS-PAGE (sodium dodecly sulfate polyacrylamide gel electrophoresis) gel have been transferred (e.g., following electrophoretic separation in the gel). Before contact of membrane with the anti-IGF1R antibody or fragment, the membrane is optionally blocked, e.g., with non-fat dry milk or the like so as to bind non-specific protein binding sites on the membrane.

(2) washing the membrane one or more times to remove unbound anti-IGF1R antibody or fragment and other unbound substances;

(3) detecting the bound anti-IGF1R antibody or fragment.

Detection of the bound antibody or fragment may be by binding the antibody or fragment with a secondary antibody (an anti-immunoglobulin antibody) which is detectably labeled and, then, detecting the presence of the secondary antibody.

The anti-IGF1R antibodies and antigen-binding fragments thereof of the invention may also be used for immunohistochemistry. Such a method forms part of the present invention and comprises, e.g.,

(1) contacting a cell to be tested for the presence of IGF1R with an anti-IGF1R antibody or antigen-binding fragment thereof of the invention;

(2) detecting the antibody or fragment on or in the cell.

If the antibody or fragment itself is detectably labeled, it can be dected directly. Alternatively, the antibody or fragment may be bound by a detectably labeled secondary antibody which is detected.

The anti-IGF1R antibodies and antigen-binding fragments thereof of the invention may also be used for in vivo tumor imaging. Such a method forms part of the present invention and may include injection of a radiolabeled anti-IGF1R antibody or antigen-binding fragment thereof of the invention into the body of a patient to be tested for the presence of a tumor which expresses IGF1R followed by nuclear imaging of the body of the patient to detect the presence of the labeled antibody or fragment e.g., at loci comprising a high concentration of the antibody or fragment which are bound to the tumor.

Imaging techniques include SPECT imaging (single photon emission computed tomography) or PET imaging (positron emissioin tomography). Labels include e.g., iodine-123 (¹²³I) and technetium-99m (^{99m}Tc), e.g., in conjunction with SPECT imaging or ¹¹C, ¹³N, ¹⁵O or ¹⁸F, e.g., in conjunction with PET imaging or Indium-111 (See e.g., Gordon et al., (2005) International Rev. Neurobiol. 67:385-440).

### EXAMPLES

The present invention is intended to exemplify the present invention and not to be a limitation thereof. Any method or composition disclosed below falls within the scope of the present invention.

### Example 1

### 9H2 and 2C6 Inhibit IGF-I and IGF-II Mediated IGF1R Kinase Activitation in KIRA Assays

MCF 7 cells were seeded in 96-well tissue culture plates (Falcon # 35-3075) at 200,000 cells/well. The testing antibody (9H2 or 2C6) supernatants or purified antibody were added to the plates and incubated for 30 minutes. Cells were then stimulated with IGF-I (7.5 ng/well in 1000 ul) for 15 minutes. An IGF only control well and no-treatment control well were included. Cells were lysed with lysis buffer (150 mM NaCl, 50 mM Hepes, 1.0% Triton X-100, 2 mM Sodium Orthovanadate, Protease Inhibitor Cocktail Complete (Roche)). Cell lysates (85 μL) were transferred from cell plate(s) wells to the prepared ELISA plate(s).

ELISA plate(s) (NUNC MAXI-SORP surface # 439-454) were prepared with 100 ng/well anti-IGF1R capture antibodies, 19D12, overnight at 4° C. Plates were washed 6× with 150 μL/well wash buffer (PBS, 0.05% Tween 20). Following incubation of lysates, ELISA plate(s) were washed 4× with 100 uL/well wash buffer, and 20 ng/well detecting Ab 4G10 in 100 μL was added. Plate(s) were on a plate shaker at room temperature for 2 hours and then washed 4× with 100 ul/well wash buffer. 100 μL/well HRP conjugated straptavidin was added to ELISA plate(s). Plate(s) were placed on a plate shaker at room temperature for 30 minutes, and then washed 4× with 100 μL/well wash buffer. TMB substrate was added to ELISA plate(s), and the plates were placed on a plate shaker at room temperature for 5-15 minutes. Following TMB incubation, 50 μL/well 1N H2SO₄ stop agent was added. Plates were read on plate reader (Molecular Devices or equivalent) at 450/650.

The data generated in these assays is set forth below in Table 2.

TABLE 2
IC50 values associated with anti-IGF1R antibodies
9H2 and 2C6 as determined with KIRA assays.

| | KIRA IGF-I | KIRA IGF-II |
|---|---|---|
| | IC50 (nM) | IC50 (nM) |
| 9H2 | 7.8 | 2.1 |
| 2C6 | 1.4 | 1.2 |

Accordingly, the present invention includes antibodies and antigen-binding fragments thereof, as described herein, which exhibit any of the IC50 values set forth in Table 2.

### Example 2

### Affinity Measurements of Antibodies 9H2 and 2C6 for IGF1R

In these assays, the affinity of the 9H2 and 2C6 antibodies for IGF1R were determined using BIACore and KineXa assays.

BIACore. Soluble IGF1R was coupled to CM5 sensor chip via amine group linkage using the Wizard control software. Immobilization levels of IGF1R were targeted at 300 Resonance Unit (RU). Recombinant human IL-5 receptor (hIL-5R) was used as reference surface for subtracting nonspecific binding and bulk refractive index. The immobilized surfaces were subsequently conditioned with several pulses of regeneration buffer (25 mM HCl+1M NaCl) prior to affinity measurement.

Association rate constant (Ka) and dissociation rate constant (Kd) of the interactions of anti-IGF1R antibody to soluble IGF1R were measured on the BIACore 3000 system using Control Software version 3.1. Anti-IGF1R antibodies were two-fold serially diluted in the HBS-EP running buffer including a zero control of HBS-EP buffer only in the concentration series. The antibodies were randomly injected in duplicate over the immobilized IGF1R and IL-5R flow cell surfaces at a flow rate 30 μl/min for 3 min at which time the injection was terminated and the amount of bound antibody measured (association). Buffer was then passed over the surface for the next 60 minutes at the same flow rate (dissociation). Following the dissociation, regeneration buffer was used to remove remaining anti-IGF1R antibody from the immobilized IGF1R surface, and the regenerated surface was then used for the next cycle of binding. Binding interaction temperature was maintained at 25° C. using the instrument temperature control function. The samples were maintained at 4° C. on a cooled sample block for the duration of the assay.

KinExA. NHS-PMMA beads, with an average diameter of 98 mm, were used in all experiments. Purified soluble IGF1R was covalently coupled to NHS-PMMA. One ml of coating solution containing 10 mg/ml sIGF1R in 50 mM sodium carbonate buffer, pH 9.6, was added to the 200 mg of NHS-PMMA beads and rocked overnight at 4° C. After incubation, the NHS-PMMA beads were allowed to settle for 3 min. The coating solution was then discarded and the beads were rinsed twice with 1 ml of 1M Tris-buffer, pH 8.0. Unreacted coupling sites and non-specific protein binding sites on the NHS-PMMA beads were blocked with 1M Tris buffer, pH 8.0, containing 10% BSA at room temperature for 2 hours. Beads were resuspended with 27 ml of PBS running buffer.

Non-specific binding (NSB) from fluorescence labeled secondary antibody and 100% signal from anti-IGF1R antibody without the presence of antigen were tested prior to affinity measurements. Using information from the NSB (0% signal) and 100% signal measurements, antibody and antigen concentrations were chosen to produce experimental signals ranging from 100% free and 0% free signals for the antibody. To measure antibody affinity, a soluble receptor was serially diluted with a fixed concentration of antibody and equilibrated. The equilibrated antigen-antibody solution sets were performed at various concentrations of antibody to obtain accurate information on both active binding site concentration and affinity. The mixture of antibody and antigen was rocked continuously for 4 hours at room temperature. The equilibrated mixture was then analyzed using the KinExA 3000.

Antibody affinity for cell surface IGF1R was determined for IGF1R transfected-293 cells using multi-curve analysis. As with the soluble receptor, experiments performed with multiple antibody concentrations were used for affinity determination. IgG concentrations of 10 μM or 160 μM were chosen for the low and high antibody concentrations for the whole cell assay. To obtain complete experimental curves, spanning the 0% and 100% free antibody signal range, the cells were subjected to a two-fold serial dilution in 1% BSA-PBS buffer and incubated with both low and high concentrations of antibodies. Once equilibrium was reached, the cells and bound antibodies were pelleted at 400 g for 10 minutes. Supernatants containing free antibody were carefully transferred into new test tubes and analyzed using the KinExA 3000.

Antibody affinity for soluble IGF1R and cell surface IGF1R was determined using the KinExA 3000 (Sapidyne Instrument Inc., Boise, Id.). A uniform and reproducible bead pack coated with IGF1R was created in the flow cell, held in place by a retention screen, and samples drawn through the bead column. As the samples rapidly passed through the column a small amount of the free antibody in the mixture was captured. The remaining unbound antibody was washed away. Captured antibody was then labeled by flowing a Cy5-conjugated goat anti-human IgG (H+L) or Cy5-conjugated goat anti-human IgG, F(ab)′₂ fragment specific over the column, followed by washed with PBS at a flow rate of 1.5 ml/min for 1 min. The signal (delta) used for calculations was the difference between the final signal or endpoint, 10 to 5 seconds from the end of each run, subtracted from the initial signal at the beginning baseline, 5 to 10 seconds into each run. All samples were run in duplicate.

The affinity of antibody 9H2 for IGF1R, as measured with the KineXa assay, was 1.24×10⁻¹⁰ M. The affinity of antibody 2C6 for IGF1R, as measured with the BiaCore assay, was 2.01×10⁻⁹M.

Accordingly, the present invention includes antibodies and antigen-binding fragments thereof comprising 9H2CDRs which exhibit a K_{D} of about 1.24×10¹⁰ M or a lower number; and antibodies and antigen-binding fragments thereof comprising 2C6 CDRs which exhibit a K_{D} of about 2.01×10⁻⁹M or a lower number.

### Example 3

### Antibody 9H2 Inhibits Anchorage Independent Cell Growth

Three milliliters of 0.6% agarose in complete MEM medium were added to each well of 6 well tissue culture plates and allowed to solidify (bottom layer). One hundred microliters of antibody 9H2 or a blank, at various concentrations, was added to culture tubes. HT29 cells (human colon adenocarcinoma grade II cell line) were harvested. Aliquots of the cells (15,000 cells) were added to the culture tubes containing the antibody and incubated at room temperature for 10-15 minutes. Three milliliters of a 0.35% agarose/complete minimal essential media (MEM) layer (top layer) were added to the antibody/cell mixture and then plated onto the solidified bottom layer. The top layer was allowed to solidify. The plates were then incubated for three weeks. MTT (3-(4,5-Dimethyl-2-Thiazolyl)-2,5-Diphenyl-2H-Tetrazolium Bromide) was added to the wells and incubated for 1-2 hours. The plates were scanned and the colonies counted and analyzed using a customized colony counter application program.

The 9H2 antibody was observed to inhibit 75% of HT29 cell growth. The results of these experiments demonstrated that an anti-IGF1R antibody can inhibit anchorage-independent growth of all three malignant cell lines tested.

Accordingly, the present invention includes antibodies and antigen-binding fragments thereof, as described herein, which inhibit anchorage independent growth, e.g., of malignant cells such as e.g., HT29 cells, for example at a level of 75% or more inhibition.

### Example 4

### Antibodies 2C6 and 9H2 Inhibit Cellular Proliferation

The ability of anti-IGF1R antibodies 2C6 and 9H2 to inhibit cellular proliferation was demonstrated in this example.

Cell Preparation. SK-N-AS (human neuroblastoma) and MCF7L (human breast adenocarcinoma) cells were cultured for several passages no greater than 80% confluency in T-75 TC treated filtered flasks. The cells were trypsinized, counted and resuspended at a concentration of 25000 cells/ml in 10% HI-FBS (heat-inactivated fetal bovine serum) RPMI medium containing NEAA (non-essential amino acids), L-Glu, MEM Vitamins and PS. 100 ul of cell suspension (2500 cells) was added to each well of a BD Falcon 96 well black, clear bottom TC treated plate. The cells were allowed to attach overnight at 37° C. The 10% RPMI was replaced with 100 ul RPML containing 2% HI-FBS containing NEAA, L-Glu, MEM Vitamins and PS in the presence or absence of the 2C6 and 9H2 anti-IGF1R antibody. The antibody concentrations used were 100 nM and a series of 1/5 dilutions down to 0.00026 nM.

Solution Preparation. All assay reagents were prepared in RPMI containing 2% HI-FBS at 20× concentration and serial diluted for a total of 10 test concentrations per treatment. Every test point was prepared in triplicate on separate assay plates. Cell proliferation was measured on Day 4 (discussed below).

Assay. Cell proliferation was measured using the Promega Cell Titer-Glo Luminescent Cell Viability Assay (Promega Corp.; Madison, Wis.). This assay provided a method for determining the number of viable cells in culture based on quantitation of ATP in the culture, which indicates the presence of metabolically active cells.

The assay reagents and assay plates were equilibrated to room temperature and prepared immediately before addition to the assay plates. One volume of CellTiter-Glo® assay reagent was added to the incubated cells in each well of the assay plate and shaken on an orbital platform. The assay reagent was shaken for at least ten minutes to allow for equilibration of the ATP reaction and to ensure total lysis of all cells in the assay plate. The reaction had a half-life of five hours but in no case was reading done later than 30 minutes after addition of reagent. Luminescence was detected on Wallac 420 Plate Reader with stacker.

The results from these experiments are shown below in Table 3. The units in the tables (proliferation index) are arbitrary and are proportional to the number of viable cells observed in the culture under each respective condition.

TABLE 3
Percentage of cellular growth inhibition
in the presence of anti-IGF1R antibodies

| | Percentage cellular |
|---|---|
| Anti-IGF1R antibody | growth inhibition |
| 2C6 | 53 (of SK-N-AS cells) |
| 2C6 | 21 (of MCF7L cells) |
| 9H2 | 42 (of MCF7L cells) |

Accordingly, the present invention includes antibodies and antigen-binding fragments thereof comprising 9H2CDRs which inhibit malignant cell growth, e.g, MCF7L growth at a level of 42% or more inhibition; and antibodies and antigen-binding fragments thereof comprising 2C6 CDRs which inhibit malignant cell growth, e.g., SK-N-AS growth at a level of 53% or more inhibition, or e.g., MCF7L cell growth at 21% or more inhibition.

### Example 5

### Anti-IGF1R Antibodies 9H2 and 2C6 Inhibit Human Neuroblastoma Tumor Growth In Vivo

In this example, the ability of antibodies 9H2 and 2C6 to inhibit the growth of human neuroblastoma cells, in vivo, was demonstrated.

Athymic nude mice were inoculated with SK-N-AS tumor cells in the right flank, subcutaneously, along with Matrigel (1:1 cells:gel). In these experiments, 5×10⁶ cells/mouse in a 1:1 mix with regular matrigel were inoculated subcutaneously. Tumor size was measured with calipers and the data was entered into the Labcat® program (Innovative Programming Associates, Inc.; Princeton, N.J.). Mice were grouped with an average body size of 100 mm³. In these experiments, mice were dosed twice per week, intraperitoneally (i.p.), with 0.1 mg of anti-IGF1R antibody 9H2 or 2C6 or with a control IgG1 antibody. Tumor size and mouse body weight was measured twice weekly after treatment.

The data gathered in these experiments is set forth below in Table 4.

TABLE 4
Average level of inhibition of neuroblastoma tumor growth inhibition
observed in mice receiving antibody therapy (n = 10).

| | control IgG1 | 9H2 | 2C6 |
|---|---|---|---|
| Mean Tumor Size (mm3) | 1608 | 278 | 542 |

Accordingly, the present invention includes antibodies and antigen-binding fragments thereof as described herein comprising 9H2CDRs or 2C6 CDRs which inhibit neuroblastoma tumor growth at a level of at least that shown in Table 4.

### Example 6

### Epitope Mapping by ELISA

In this example, the ability of antibodies 2C6 and 9H2 to compete with anti-IGF1R antibody 19D12 was demonstrated in a competitive sR ELISA.

These assays were performed as follows: Falcon 96 well assay plates were coated overnight at 4° C. with 2.0 ug/ml recombinant human soluble IGF1R fragment (R&D Systems) in 1×PBS, 50 ul/well. The plate was emptied and blocked in 1×PBS/0.05% Tween 20+5% chicken serum (100 ul/well) for one hour at room temperature. The plate was then emptied and washed 3× using 1×PBST.

Test antibodies were diluted in 1×PBST+5% ChS and added to wells (100 uL/well total) for one hour at RT (biotinylated 19D12 LCF/HCA (see U.S. Pat. No. 7,217,796) was diluted to 400 ng/mL and 50 uL was added to wells; competing Abs were diluted to a concentration 2× the desired final concentration and 50 uL of each dilution were added per well). The plate was then washed 3×. Streptavidin-HRP secondary antibody was diluted 1:2000 in 1×PBST+5% ChS and added to wells (100 uL/well) for one hour at RT. The plate was washed 3× again. The plate was developed for 30 min-1 hour using citrate phosphate buffer pH 4/ABTS/H2O₂. The plate was read with Molecular Devices software (405-490), 4-parameter fit. The data generated in these assay are set forth below in Table 5.

TABLE 5
Optical density measurements observed during competitive
ELISA assay and percentage of inhibition of binding of
biotinylated 19D12 antibody to the IGF1R fragment.

| | | | | |
|---|---|---|---|---|
| ug/ml | 19D12 | MAB391 | 2C6 | 9H2 |

OD

| | | | | |
|---|---|---|---|---|
| 8 | −0.0045 | 0.045 | | 0.3525 |
| 4 | −0.0025 | 0.0725 | | 0.4315 |
| 2 | −0.0025 | 0.163 | 1.33 | 0.5875 |
| 1 | 0.001 | 0.3555 | 1.355 | 0.7575 |
| 0.5 | 0.021 | 0.628 | 1.392 | 0.969 |
| 0.25 | 0.169 | 0.981 | 1.369 | 1.1885 |
| 0.125 | 0.443 | 1.1555 | 1.3205 | 1.2045 |
| 0.0625 | 1.0005 | 1.244 | | 1.3155 |
| 0.03125 | 1.1025 | 1.4075 | | 1.3395 |
| 0.015625 | 1.2485 | 1.254 | | 1.3945 |
| 0.007813 | 1.2265 | 1.306 | | 1.37 |

% Inhibition

| | | | | |
|---|---|---|---|---|
| 8 | 100.352 | 96.47979 | | 74.2732 |
| 4 | 100.1956 | 94.32855 | | 68.50748 |
| 2 | 100.1956 | 87.24902 | −4.04172 | 57.122 |
| 1 | 99.92177 | 72.19035 | −5.99739 | 44.71475 |
| 0.5 | 98.35724 | 50.87353 | −8.89179 | 29.27868 |
| 0.25 | 86.77966 | 23.25945 | −7.09257 | 13.25873 |
| 0.125 | 65.3455 | 9.608866 | −3.29857 | 12.09099 |
| 0.0625 | 21.73403 | 2.685789 | | 3.989782 |
| 0.03125 | 13.75489 | −10.1043 | | 2.238171 |
| 0.015625 | 2.333768 | 1.90352 | | −1.77594 |
| 0.007813 | 4.054759 | −2.16428 | | 0.012164 |

These data demonstrated that 2C6 antibody did not complete with 19D12, and therefore it recognizes an epitope that is not overlapping with 19D12. 9H2 or the commercially available anti-IGF1R antibody, MB391, competed for IGF1R binding with the 19D12 antibody. 9H2 recognizes an overlapping epitope with 19D12.

Accordingly, the present invention includes antibodies and antigen-binding fragments thereof as described herein comprising 9H2CDRs which compete with 19D12 for IGF1R binding; or comprising 2C6 CDRs which do not compete with 19D12 for IGF1R binding.

### Example 7

### Anti-IGF1R Antibody 9H2 Down-Regulates Cellular Expression of IGF1R

In this example, the level of cellular expression of IGF1R was shown to decrease in a manner dependent on exposure to anti-IGF1R antibody 9H2.

H322 cells were treated with various concentrations of 9H2 antibody (lane 2: 20 nM, lane 3: 2 nM, lane 4: 0.2 nM, lane 5: 0.02 nM) for four hours. 50 μg of each cell lysate was separated by a 10% SDS-PAGE and transferred to a nitrocellulose blot. The blot was probed with anti-IGF1R antibody.

The blot demonstrated that the intensity of bands corresponding to IGF1R decreased as the concentration of antibody to which the cells were exposed increased.

Accordingly, the present invention includes antibodies and antigen-binding fragments thereof as described herein comprising 9H2 or 2C6 CDRs which down regulate IGF1R expression in malignant cells, e.g., in H322 cells.

### Example 8

### Identification and Isolation of 9H2 and 2C6

In this example, the isolation and identification of the 9H2 and 2C6 antibodies and the generation of hybridomas which express the antibodies is described.

Antigen. Mice were immunized with two forms of antigen in alternation: (1) live cells (IGF1R transfected HEK293 cells) and (2) purified protein (sIGF1R; a recombinant protein encompassing the α-subunit and the extracellular domain of IGF1R; amino acid residues Met 1-Asn 932) (Ullrich et al. (1986) EMBO J. 5:2503-2512).

Transgenic Mice. HuMab Mice (Medarex, Inc.; Princeton, N.J.) were housed in filter cages and were evaluated to be in good physical condition at the time of immunization, at the time of the bleeds and on the day fusions were produced. The mice were of the (CMD); (Hco7) 11952; (JKD); (KCo5) 9272 genotype. Immunization Procedure. Mice were immunized with two forms of the antigen in alternation: 1.0×10⁷ IGF1R transfected HEK293 cells in saline and sIGF1R (20-40 ug). CFA adjuvant and sIGF1R (20-40 μg) were injected on Days 15, 43, 112, 128 and 129. Antibody titers were measured on Days 37, 54, 103 and 126. Fusions were performed on Day 131.

Hybridoma Preparation. The SP2/0-AG14 myeloma cell line (ATCC CRL 1581) was used for the fusions. High Glucose DMEM containing 10% FBS, antibiotic-antimycotic (100×), and 0.1% L-glutamine was used to culture myeloma cells. Additional media supplements were added to the hybridoma growth media which included: 5% Origen—Hybridoma Cloning Factor (Fischer Scientific; Suwanee, Ga.), 4.5×10⁻⁴ M sodium Pyruvate, HAT (1.0×10⁻⁴ M Hypoxanthine, 4.0×10⁻⁷ M Aminopterin, 1.6×10⁻⁵ M Thymidine), or HT (1.0×10⁻⁴ M Hypoxanthine, 1.6×10⁻⁵ M Thymidine); and characterized fetal bovine serum.

The spleen from a mouse was normal in size and yielded viable cells. The splenocytes were fused according to standard procedures.

Screening of hybridomas. An initial ELISA screen for human IgGκ antibodies was performed 7-10 days post-fusion according to the following procedure:
(1) Coated plate overnight with anti-hu-κ, I μg/mL or anti-hu-γ, 1 μg/mL in 1×PBS, 50 μL/well. Stored in refrigerator.
(2) Emptied plate and blocked plate in 1×PBST (PBS with Tween)+5% chicken serum for 1 hour at room temperature (100 μL/well).
(3) Emptied plate and washed manually with wash bottle (3×) or plate washer (3×) using 1×PBST.
(4) Standards were used for testing production level of the clones. Made dilutions with unknowns (1:10 in first well and dilute 2 fold across plate). Hu-IgG standards start at 1000 ng/mL and dilute 2 fold across plate.
Left a few wells for blanks: 1×PBST+5% chicken serum which is used for dilutions, 100 μL/well. Incubated at room temperature for 1 hour. Fusion screens and subclones were tested diluted 1:2 in blocking buffer.
   5. Repeated wash step #3.
   6. Diluted secondary antibody HRP(horse radish peroxidase)-anti-hu IgG-Fc reagent 1:5000 or HRP-anti-hu-κ in 1×PBST+5% chicken serum, added 100 μL/well. Incubated 1 hour at room temperature.
   7. Repeated wash step#3. (2×)
   8. Developed plate using 10 ml citrate phosphate buffer pH4.0, 80 μL ABTS, 8 μL H₂0₂ per plate.
   9. Incubated 30 minutes to 1 hour at room temperature. Read plate at OD_{415 nm-490 nm}.

Solutions:

1×PBST=1×PBS+0.05% tween-20
Citrate phosphate buffer=21 gm/L citric acid, 14.2 gm/L disodium hydrogen phosphate (anhydrous); pH4.0
ABTS=27.8 mg/mL 2,2′-azino-bis(3-ethylbenz-thiazoline-6-sulfonic acid) diammonium salt in citrate buffer, freeze 1 mL aliquots.
Plate=96 well assay plate.

A positive ELISA signal was detected in the wells corresponding to hybridomas 9H2 and 2C6, demonstrating that these hybridomas produced human IgG antibodies.

Hybridoma supernatants corresponding to human IgGκ positive wells were then screened on soluble IGF1R coated ELISA plates according to the following procedure:

(1) Coated plate overnight with IGF1R (1.0 μg/mL) in 1×PBS, 50 μL/well. Stored in refrigerator. Five milliliters needed for coating plate.
(2) Emptied plate and blocked plate in 1×PBST+5% chicken serum for 1 hour at room temperature (100 μL/well).
(3) Emptied plate and washed manually with wash bottle (3×) or plate washer (3×) using 1×PBST.
(4) Used blocking buffer as diluent. Test sera, beginning at 1:50 dilution in the top row of the plate and diluted 2 fold/row down the plate (7×). Incubated at room temperature 1 hour. For subclone screening, a 1:1 dilution of culture supernatant in blocking buffer was used as starting material.
(5) Repeated wash step #3.
(6) Diluted secondary HRP-anti-hu IgG-Fc specific and/or HRP-anti-hu-K reagent 1:2500-5000 in 1×PBST+5% chicken serum, added 100 μL/well. Incubated 1 hour at room temperature.
(7) Repeat wash step#3. (2×)
(8) Developed plate using 10 mL citrate-phosphate buffer pH4.0, 804 ABTS, 8 μL H₂0₂ per plate.
(9) Incubated 30 minutes to 1 hour at room temperature. Read plate at OD_{415 nm-490 nm}. Considered twice above background titer limit.

In these assays, hybridomas 9H2 and 2C6 produced a positive ELISA signal. These data demonstrated that the hybridomas produced antibodies which can bind to soluble IGF1R.

Antigen positive hybridomas were then transferred to 24 well plates, and eventually to tissue culture flasks. IGF1R specific hybridomas were subcloned by limiting dilution to assure monoclonality. Antigen positive hybridomas were preserved at several stages in the development process by freezing cells in Origen DMSO freeze medium (Fischer Scientific; Suwanee, Ga.).

The isotypes of 9H2 and 2C6 were determined according to the following procedure:

(1) Coated plate overnight in refrigerator at I μg/ml soluble IGF1R in 1×PBS, 504/well. Emptied plate.
(2) Added 1×PBST+5% chicken serum for 1 hour at room temperature. (100 μL/well). Emptied plate.
(3) Used blocking buffer as a diluent, added supernatant or purified material to be tested in 1 well per secondary antibody to be tested-504/well. Incubated for 90 minutes at room temperature. Emptied plate.
(4) Emptied plate and washed manually with wash bottle (3×) or plate washer (3×) using 1×PBST.
(5) Using blocking buffer as a diluent, added secondary antibodies:

HRP-anti-hu-gamma;

HRP-anti-hu kappa;

HRP-anti-human IgGl; or

HRP-anti-human IgG3

diluted 1:1000.

Incubated for 45 minutes at room temperature. Emptied plate.

(6) Repeated wash step #4 (3×).
(7) Developed plate using 10 mL citrate-phosphate buffer pH 4.0, 80 μL ABTS, 8 μL H2O₂ per plate.
(8) Incubated 30 minutes to 1 hour at room temperature. Read plate at OD₄₁₅ₙₘ₋₄₉₀ₙₘ.

These data demonstrated that the 9H2 and 2C6 heavy chains were of the IgG1 subtype and the 9H2 and 2C6 light chain was of the kappa subtype.

The present invention is not to be limited in scope by the specific embodiments described herein. Indeed, various modifications of the invention in addition to those described herein will become apparent to those skilled in the art from the foregoing description. Such modifications are intended to fall within the scope of the appended claims.

Patents, patent applications, publications, product descriptions, and protocols are cited throughout this application, the disclosures of which are incorporated herein by reference in their entireties for all purposes.

We claim:

1. An isolated antibody or antigen-binding fragment thereof comprising one or more members selected from the group consisting of:
(a) CDR-H1, CDR-H2 and CDR-H3 of the variable region of antibody 2C6;
(b) CDR-H1, CDR-H2 and CDR-H3 of the variable region of antibody 9H2;
(c) CDR-L1, CDR-L2 and CDR-L3 of the variable region of antibody 2C6; and
(d) CDR-L1, CDR-L2 and CDR-L3 of the variable region of antibody 9H2.

2. The antibody of claim 1 which is monoclonal.

3. The antibody of claim 1 which is a labeled antibody, bivalent antibody, a polyclonal antibody, a bispecific antibody, a chimeric antibody, a recombinant antibody, an anti-idiotypic antibody, a humanized antibody or a bispecific antibody.

4. The fragment of claim 1 which is a camelized single domain antibody, a diabody, an scfv, an scfv dimer, a dsfv, a (dsfv)₂, a dsFv-dsfv′, a bispecific ds diabody, an Fv, an Fab, an Fab′, an F(ab′)₂, or a domain antibody.

5. The antibody or fragment of claim 1 which is linked to a constant region.

6. The antibody or fragment of claim 5 wherein the constant region is a κ light chain, γ1 heavy chain, γ2 heavy chain, γ3 heavy chain or γ4 heavy chain.

7. A pharmaceutical composition comprising the antibody or fragment of claim 1 in association with a pharmaceutically acceptable carrier.

8. A composition comprising the antibody or fragment of claim 1 in association with one or more further chemotherapeutic agents.

9. The composition of claim 8 comprising the antibody or fragment in association with one or more members selected from the group consisting of: everolimus, trabectedin, abraxane, TLK 286, AV-299, DN-101, pazopanib, GSK690693, RTA 744, ON 0910.Na, AZD 6244 (ARRY-142886), AMN-107, TKI-258, GSK461364, AZD 1152, enzastaurin, vandetanib, ARQ-197, MK-0457, MLN8054, PHA-739358, R-763, AT-9263, a FLT-3 inhibitor, a VEGFR inhibitor, an EGFR TK inhibitor, an aurora kinase inhibitor, a PIK-1 modulator, a Bcl-2 inhibitor, an HDAC inhibitor, a c-MET inhibitor, a PARP inhibitor, a Cdk inhibitor, an EGFR TK inhibitor, an IGFR-TK inhibitor, an anti-HGF antibody, a PI3 kinase inhibitors, an AKT inhibitor, a JAK/STAT inhibitor, a checkpoint-1 or 2 inhibitor, a focal adhesion kinase inhibitor, a Map kinase kinase (mek) inhibitor, a VEGF trap antibody, pemetrexed, erlotinib, dasatanib, nilotinib, decatanib, panitumumab, amrubicin, oregovomab, Lep-etu, nolatrexed, azd2171, batabulin, ofatumumab, zanolimumab, edotecarin, tetrandrine, rubitecan, tesmilifene, oblimersen, ticilimumab, ipilimumab, gossypol, Bio 111, 131-I-TM-601, ALT-110, BIO 140, CC 8490, cilengitide, gimatecan, IL13-PE38QQR, INO 1001, IPdR, KRX-0402, lucanthone, LY 317615, neuradiab, vitespan, Rta 744, Sdx 102, talampanel, atrasentan, Xr 311, romidepsin, ADS-100380, 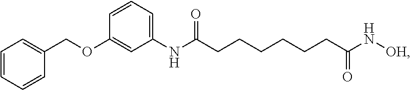
CG-781, CG-1521; 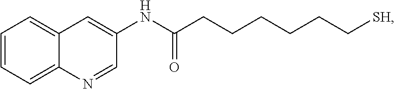
SB-556629, chlamydocin, JNJ-16241199, 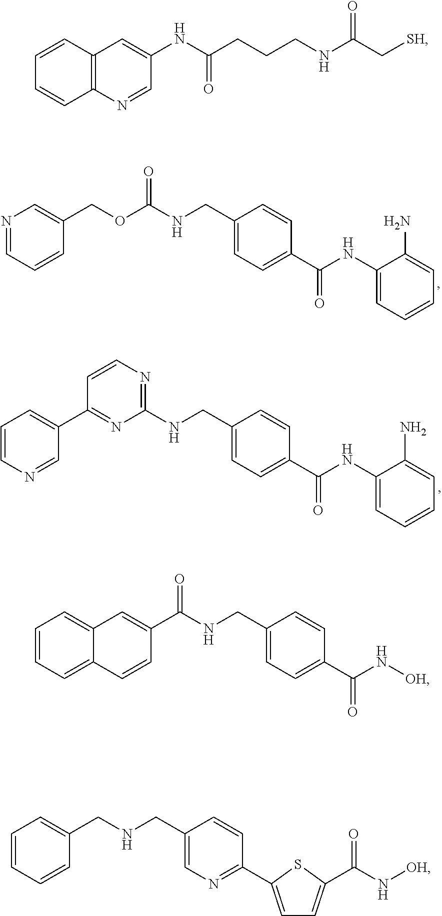
vorinostat, etoposide, gemcitabine, doxorubicin, liposomal doxorubicin, 5′-deoxy-5-fluorouridine, vincristine, temozolomide, ZK-304709, seliciclib; PD0325901, AZD-6244, capecitabine, L-Glutamic acid, N-[4-[2-(2-amino-4,7-dihydro-4-oxo-1H-pyrrolo[2,3-d]pyrimidin-5-yl)ethyl]benzoyl]-, disodium salt, heptahydrate, camptothecin, irinotecan; a combination of irinotecan, 5-fluorouracil and leucovorin; PEG-labeled irinotecan, FOLFOX regimen, tamoxifen, toremifene citrate, anastrazole, exemestane, letrozole, DES(diethylstilbestrol), estradiol, estrogen, conjugated estrogen, bevacizumab, IMC-1C11, CHIR-258, 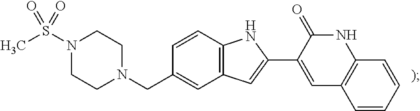
3-[5-(methylsulfonylpiperadinemethyl)-indolyl]-quinolone, vatalanib, AG-013736, AVE-0005, the acetate salt of [D-Ser(Bu t)6, Azgly 10] (pyro-Glu-His-Trp-Ser-Tyr-D-Ser(Bu t)-Leu-Arg-Pro-Azgly-NH2 acetate [C₅₉H84N₁₈O₁₄.(C₂H4O₂)ₓ where x=1 to 2.4], goserelin acetate, leuprolide acetate, triptorelin pamoate, sunitinib, sunitinib malate, medroxyprogesterone acetate, hydroxyprogesterone caproate, megestrol acetate, raloxifene, bicalutamide, flutamide, nilutamide, megestrol acetate, CP-724714; TAK-165, HKI-272, erlotinib, lapatanib, canertinib, ABX-EGF antibody, erbitux, EKB-569, PKI-166, GW-572016, lonafarnib, 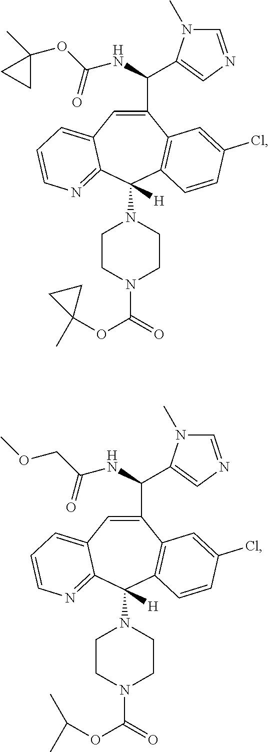
BMS-214662, tipifarnib; amifostine, NVP-LAQ824, suberoyl analide hydroxamic acid, valproic acid, trichostatin A, FK-228, SU11248, sorafenib, KRN951, aminoglutethimide, amsacrine, anagrelide, L-asparaginase, Bacillus Calmette-Guerin (BCG) vaccine, bleomycin, buserelin, busulfan, carboplatin, carmustine, chlorambucil, cisplatin, cladribine, clodronate, cyclophosphamide, cyproterone, cytarabine, dacarbazine, dactinomycin, daunorubicin, diethylstilbestrol, epirubicin, fludarabine, fludrocortisone, fluoxymesterone, flutamide, hydroxyurea, idarubicin, ifosfamide, imatinib, leucovorin, leuprolide, levamisole, lomustine, mechlorethamine, melphalan, 6-mercaptopurine, mesna, methotrexate, mitomycin, mitotane, mitoxantrone, nilutamide, octreotide, oxaliplatin, pamidronate, pentostatin, plicamycin, porfimer, procarbazine, raltitrexed, rituximab, streptozocin, teniposide, testosterone, thalidomide, thioguanine, thiotepa, tretinoin, vindesine, 13-cis-retinoic acid, phenylalanine mustard, uracil mustard, estramustine, altretamine, floxuridine, 5-deooxyuridine, cytosine arabinoside, 6-mercaptopurine, deoxycoformycin, calcitriol, valrubicin, mithramycin, vinblastine, vinorelbine, topotecan, razoxin, marimastat, COL-3, neovastat, BMS-275291, squalamine, endostatin, SU5416, SU6668, EMD121974, interleukin-12, IM862, angiostatin, vitaxin, droloxifene, idoxyfene, spironolactone, finasteride, cimitidine, trastuzumab, denileukin diftitox, gefitinib, bortezimib, paclitaxel, cremophor-free paclitaxel, docetaxel, epithilone B, BMS-247550, BMS-310705, droloxifene, 4-hydroxytamoxifen, pipendoxifene, ERA-923, arzoxifene, fulvestrant, acolbifene, lasofoxifene, idoxifene, TSE-424, HMR-3339, ZK186619, topotecan, PTK787/ZK 222584, VX-745, PD 184352, rapamycin, 40-O-(2-hydroxyethyl)-rapamycin, temsirolimus, AP-23573, RAD001, ABT-578, BC-210, LY294002, LY292223, LY292696, LY293684, LY293646, wortmannin, ZM336372, L-779,450, PEG-filgrastim, darbepoetin, 5-fluorouracil, erythropoietin, granulocyte colony-stimulating factor, zolendronate, prednisone, cetuximab, granulocyte macrophage colony-stimulating factor, histrelin, pegylated interferon alfa-2a, interferon alfa-2a, pegylated interferon alfa-2b, interferon alfa-2b, azacitidine, PEG-L-asparaginase, lenalidomide, gemtuzumab, hydrocortisone, interleukin-11, dexrazoxane, alemtuzumab, all-transretinoic acid, ketoconazole, interleukin-2, megestrol, immune globulin, nitrogen mustard, methylprednisolone, ibritgumomab tiuxetan, androgens, decitabine, hexamethylmelamine, bexarotene, tositumomab, arsenic trioxide, cortisone, editronate, mitotane, cyclosporine, liposomal daunorubicin, Edwina-asparaginase, strontium 89, casopitant, netupitant, an NK-1 receptor antagonists, palonosetron, aprepitant, diphenhydramine, hydroxyzine, metoclopramide, lorazepam, alprazolam, haloperidol, droperidol, dronabinol, dexamethasone, methylprednisolone, prochlorperazine, granisetron, ondansetron, dolasetron, tropisetron, pegfilgrastim, erythropoietin, epoetin alfa and darbepoetin alfa.

10. The antibody or fragment of claim 1 which is bound to IGF1R or a fragment thereof.

11. An isolated antibody or antigen-binding fragment thereof comprising one or more members selected from the group consisting of:
(a) 2C6 CDR-H1 defined by SEQ ID NO: 2, 2C6 CDR-H2 defined by SEQ ID NO: 3 and 2C6 CDR-H3 defined by SEQ ID NO: 4;
(b) 9H2CDR-H1 defined by SEQ ID NO: 10, 9H2CDR-H2 defined by SEQ ID NO: 11 and 9H2CDR-H3 defined by SEQ ID NO: 12;
(c) 2C6 CDR-L1 defined by SEQ ID NO: 6, 2C6 CDR-L2 defined by SEQ ID NO: 7 and 2C6 CDR-L3 defined by SEQ ID NO: 8; and
(d) 9H2CDR-L1 defined by SEQ ID NO: 14, 9H2CDR-L2 defined by SEQ ID NO: 15 and 9H2CDR-L3 defined by SEQ ID NO: 16.

12. The antibody of claim 11 which is monoclonal.

13. The antibody of claim 11 which is a labeled antibody, bivalent antibody, a polyclonal antibody, a bispecific antibody, a chimeric antibody, a recombinant antibody, an anti-idiotypic antibody, a humanized antibody or a bispecific antibody.

14. The fragment of claim 11 which is a camelized single domain antibody, a diabody, an scfv, an scfv dimer, a dsfv, a (dsfv)₂, a dsFv-dsfv', a bispecific ds diabody, an Fv, an Fab, an Fab′, an F(ab′)₂, or a domain antibody.

15. The antibody or fragment of claim 11 which is linked to a constant region.

16. The antibody or fragment of claim 15 wherein the constant region is a κ light chain, γ1 heavy chain, γ2 heavy chain, γ3 heavy chain or γ4 heavy chain.

17. A pharmaceutical composition comprising the antibody or fragment of claim 11 in association with a pharmaceutically acceptable carrier.

18. A composition comprising the antibody or fragment of claim 11 in association with one or more further chemotherapeutic agents.

19. The composition of claim 18 comprising the antibody or fragment in association with one or more members selected from the group consisting of: everolimus, trabectedin, abraxane, TLK 286, AV-299, DN-101, pazopanib, GSK690693, RTA 744, ON 0910.Na, AZD 6244 (ARRY-142886), AMN-107, TKI-258, GSK461364, AZD 1152, enzastaurin, vandetanib, ARQ-197, MK-0457, MLN8054, PHA-739358, R-763, AT-9263, a FLT-3 inhibitor, a VEGFR inhibitor, an EGFR TK inhibitor, an aurora kinase inhibitor, a PIK-1 modulator, a Bcl-2 inhibitor, an HDAC inhibitor, a c-MET inhibitor, a PARP inhibitor, a Cdk inhibitor, an EGFR TK inhibitor, an IGFR-TK inhibitor, an anti-HGF antibody, a PI3 kinase inhibitors, an AKT inhibitor, a JAK/STAT inhibitor, a checkpoint-1 or 2 inhibitor, a focal adhesion kinase inhibitor, a Map kinase kinase (mek) inhibitor, a VEGF trap antibody, pemetrexed, erlotinib, dasatanib, nilotinib, decatanib, panitumumab, amrubicin, oregovomab, Lep-etu, nolatrexed, azd2171, batabulin, ofatumumab, zanolimumab, edotecarin, tetrandrine, rubitecan, tesmilifene, oblimersen, ticilimumab, ipilimumab, gossypol, Bio 111, 131-I-TM-601, ALT-110, BIO 140, CC 8490, cilengitide, gimatecan, IL13-PE38QQR, INO 1001, IPdR, KRX-0402, lucanthone, LY 317615, neuradiab, vitespan, Rta 744, Sdx 102, talampanel, atrasentan, Xr 311, romidepsin, ADS-100380, 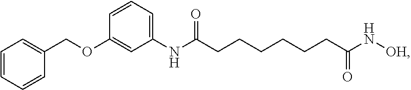
CG-781, CG-1521, 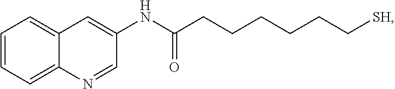
SB-556629, chlamydocin, JNJ-16241199, 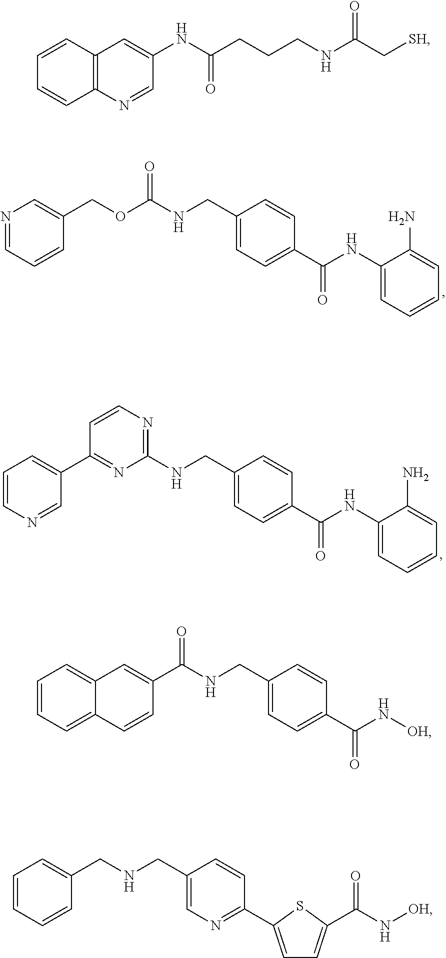
vorinostat, etoposide, gemcitabine, doxorubicin, liposomal doxorubicin, 5′-deoxy-5-fluorouridine, vincristine, temozolomide, ZK-304709, seliciclib; PD0325901, AZD-6244, capecitabine, L-Glutamic acid, N-[4-[2-(2-amino-4,7-dihydro-4-oxo-1H-pyrrolo[2,3-d]pyrimidin-5-yl)ethyl]benzoyl]-, disodium salt, heptahydrate, camptothecin, irinotecan; a combination of irinotecan, 5-fluorouracil and leucovorin; PEG-labeled irinotecan, FOLFOX regimen, tamoxifen, toremifene citrate, anastrazole, exemestane, letrozole, DES(diethylstilbestrol), estradiol, estrogen, conjugated estrogen, bevacizumab, IMC-1C11, CHIR-258, 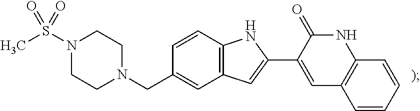
3-[5-(methylsulfonylpiperadinemethyl)-indolyl]-quinolone, vatalanib, AG-013736, AVE-0005, the acetate salt of [D-Ser(Bu t)6, Azgly 10] (pyro-Glu-His-Trp-Ser-Tyr-D-Ser(Bu t)-Leu-Arg-Pro-Azgly-NH2 acetate [C₅₉H84N₁₈O₁₄.(C₂H₄O₂)ₓ where x=1 to 2.4], goserelin acetate, leuprolide acetate, triptorelin pamoate, sunitinib, sunitinib malate, medroxyprogesterone acetate, hydroxyprogesterone caproate, megestrol acetate, raloxifene, bicalutamide, flutamide, nilutamide, megestrol acetate, CP-724714; TAK-165, HKI-272, erlotinib, lapatanib, canertinib, ABX-EGF antibody, erbitux, EKB-569, PKI-166, GW-572016, lonafarnib, 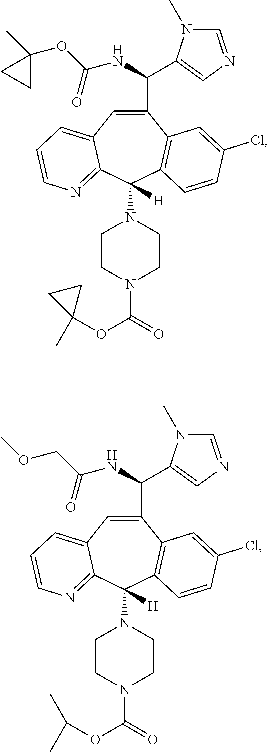
BMS-214662, tipifarnib; amifostine, NVP-LAQ824, suberoyl analide hydroxamic acid, valproic acid, trichostatin A, FK-228, SU11248, sorafenib, KRN951, aminoglutethimide, amsacrine, anagrelide, L-asparaginase, Bacillus Calmette-Guerin (BCG) vaccine, bleomycin, buserelin, busulfan, carboplatin, carmustine, chlorambucil, cisplatin, cladribine, clodronate, cyclophosphamide, cyproterone, cytarabine, dacarbazine, dactinomycin, daunorubicin, diethylstilbestrol, epirubicin, fludarabine, fludrocortisone, fluoxymesterone, flutamide, hydroxyurea, idarubicin, ifosfamide, imatinib, leucovorin, leuprolide, levamisole, lomustine, mechlorethamine, melphalan, 6-mercaptopurine, mesna, methotrexate, mitomycin, mitotane, mitoxantrone, nilutamide, octreotide, oxaliplatin, pamidronate, pentostatin, plicamycin, porfimer, procarbazine, raltitrexed, rituximab, streptozocin, teniposide, testosterone, thalidomide, thioguanine, thiotepa, tretinoin, vindesine, 13-cis-retinoic acid, phenylalanine mustard, uracil mustard, estramustine, altretamine, floxuridine, 5-deooxyuridine, cytosine arabinoside, 6-mercaptopurine, deoxycoformycin, calcitriol, valrubicin, mithramycin, vinblastine, vinorelbine, topotecan, razoxin, marimastat, COL-3, neovastat, BMS-275291, squalamine, endostatin, SU5416, SU6668, EMD121974, interleukin-12, IM862, angiostatin, vitaxin, droloxifene, idoxyfene, spironolactone, finasteride, cimitidine, trastuzumab, denileukin diftitox, gefitinib, bortezimib, paclitaxel, cremophor-free paclitaxel, docetaxel, epithilone B, BMS-247550, BMS-310705, droloxifene, 4-hydroxytamoxifen, pipendoxifene, ERA-923, arzoxifene, fulvestrant, acolbifene, lasofoxifene, idoxifene, TSE-424, HMR-3339, ZK186619, topotecan, PTK787/ZK 222584, VX-745, PD 184352, rapamycin, 40-O-(2-hydroxyethyl)-rapamycin, temsirolimus, AP-23573, RAD001, ABT-578, BC-210, LY294002, LY292223, LY292696, LY293684, LY293646, wortmannin, ZM336372, L-779,450, PEG-filgrastim, darbepoetin, 5-fluorouracil, erythropoietin, granulocyte colony-stimulating factor, zolendronate, prednisone, cetuximab, granulocyte macrophage colony-stimulating factor, histrelin, pegylated interferon alfa-2a, interferon alfa-2a, pegylated interferon alfa-2b, interferon alfa-2b, azacitidine, PEG-L-asparaginase, lenalidomide, gemtuzumab, hydrocortisone, interleukin-11, dexrazoxane, alemtuzumab, all-transretinoic acid, ketoconazole, interleukin-2, megestrol, immune globulin, nitrogen mustard, methylprednisolone, ibritgumomab tiuxetan, androgens, decitabine, hexamethylmelamine, bexarotene, tositumomab, arsenic trioxide, cortisone, editronate, mitotane, cyclosporine, liposomal daunorubicin, Edwina-asparaginase, strontium 89, casopitant, netupitant, an NK-1 receptor antagonists, palonosetron, aprepitant, diphenhydramine, hydroxyzine, metoclopramide, lorazepam, alprazolam, haloperidol, droperidol, dronabinol, dexamethasone, methylprednisolone, prochlorperazine, granisetron, ondansetron, dolasetron, tropisetron, pegfilgrastim, erythropoietin, epoetin alfa and darbepoetin alfa.

20. The antibody or fragment of claim 11 which is bound to IGF1R or a fragment thereof.

21. An isolated antibody or antigen-binding fragment thereof comprising one or more members selected from the group consisting of:
(a) a mature heavy immunoglobulin chain variable region of SEQ ID NO: 1;
(b) a mature light immunoglobulin chain variable region of SEQ ID NO: 5;
(c) a mature heavy immunoglobulin chain variable region of SEQ ID NO: 9; and
(d) a mature light immunoglobulin chain variable region of SEQ ID NO: 13.

22. The antibody of claim 21 which is monoclonal.

23. The antibody of claim 21 which is a labeled antibody, bivalent antibody, a polyclonal antibody, a bispecific antibody, a chimeric antibody, a recombinant antibody, an anti-idiotypic antibody, a humanized antibody or a bispecific antibody.

24. The fragment of claim 21 which is a camelized single domain antibody, a diabody, an scfv, an scfv dimer, a dsfv, a (dsfv)₂, a dsFv-dsfv′, a bispecific ds diabody, an Fv, an Fab, an Fab′, an F(ab′)₂, or a domain antibody.

25. The antibody or fragment of claim 21 which is linked to a constant region.

26. The antibody or fragment of claim 25 wherein the constant region is a κ light chain, γ1 heavy chain, γ2 heavy chain, γ3 heavy chain or γ4 heavy chain.

27. A pharmaceutical composition comprising the antibody or fragment of claim 21 in association with a pharmaceutically acceptable carrier.

28. A composition comprising the antibody or fragment of claim 21 in association with one or more further chemotherapeutic agents.

29. The composition of claim 28 comprising the antibody or fragment in association with one or more members selected from the group consisting of: everolimus, trabectedin, abraxane, TLK 286, AV-299, DN-101, pazopanib, GSK690693, RTA 744, ON 0910.Na, AZD 6244 (ARRY-142886), AMN-107, TKI-258, GSK461364, AZD 1152, enzastaurin, vandetanib, ARQ-197, MK-0457, MLN8054, PHA-739358, R-763, AT-9263, a FLT-3 inhibitor, a VEGFR inhibitor, an EGFR TK inhibitor, an aurora kinase inhibitor, a PIK-1 modulator, a Bcl-2 inhibitor, an HDAC inhibitor, a c-MET inhibitor, a PARP inhibitor, a Cdk inhibitor, an EGFR TK inhibitor, an IGFR-TK inhibitor, an anti-HGF antibody, a PI3 kinase inhibitors, an AKT inhibitor, a JAK/STAT inhibitor, a checkpoint-1 or 2 inhibitor, a focal adhesion kinase inhibitor, a Map kinase kinase (mek) inhibitor, a VEGF trap antibody, pemetrexed, erlotinib, dasatanib, nilotinib, decatanib, panitumumab, amrubicin, oregovomab, Lep-etu, nolatrexed, azd2171, batabulin, ofatumumab, zanolimumab, edotecarin, tetrandrine, rubitecan, tesmilifene, oblimersen, ticilimumab, ipilimumab, gossypol, Bio 111, 131-I-TM-601, ALT-110, BIO 140, CC 8490, cilengitide, gimatecan, IL13-PE38QQR, INO 1001, IPdR, KRX-0402, lucanthone, LY 317615, neuradiab, vitespan, Rta 744, Sdx 102, talampanel, atrasentan, Xr 311, romidepsin, ADS-100380, 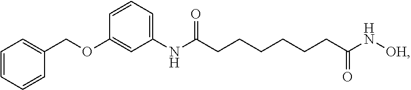
CG-781, CG-1521, 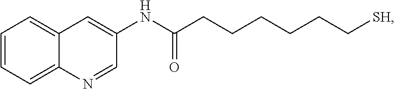
SB-556629, chlamydocin, JNJ-16241199, 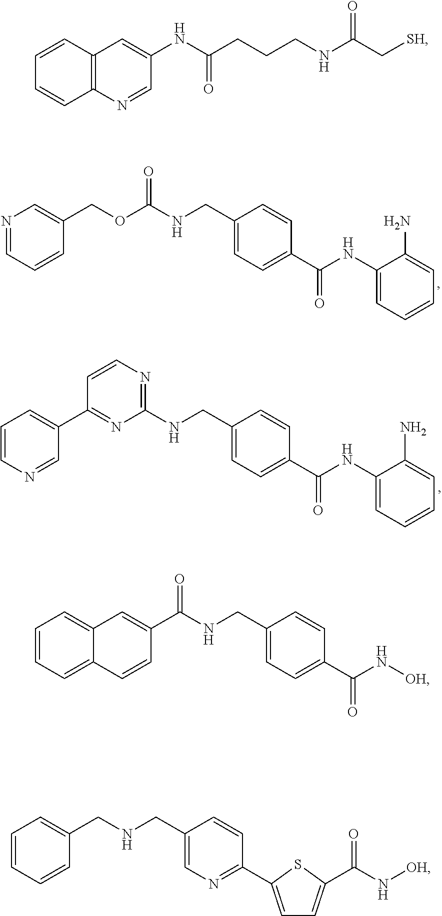
vorinostat, etoposide, gemcitabine, doxorubicin, liposomal doxorubicin, 5′-deoxy-5-fluorouridine, vincristine, temozolomide, ZK-304709, seliciclib; PD0325901, AZD-6244, capecitabine, L-Glutamic acid, N-[4-[2-(2-amino-4,7-dihydro-4-oxo-1H-pyrrolo[2,3-d]pyrimidin-5-yl)ethyl]benzoyl]-, disodium salt, heptahydrate, camptothecin, irinotecan; a combination of irinotecan, 5-fluorouracil and leucovorin; PEG-labeled irinotecan, FOLFOX regimen, tamoxifen, toremifene citrate, anastrazole, exemestane, letrozole, DES(diethylstilbestrol), estradiol, estrogen, conjugated estrogen, bevacizumab, IMC-1C11, CHIR-258, 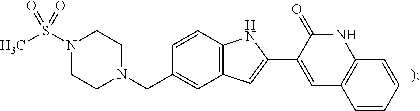
3-[5-(methylsulfonylpiperadinemethyl)-indolyl]-quinolone, vatalanib, AG-013736, AVE-0005, the acetate salt of [D-Ser(Bu t)6, Azgly 10] (pyro-Glu-His-Trp-Ser-Tyr-D-Ser(Bu t)-Leu-Arg-Pro-Azgly-NH2 acetate [C₅₉H84N₁₈O₁₄.(C₂H4O₂)ₓ where x=1 to 2.4], goserelin acetate, leuprolide acetate, triptorelin pamoate, sunitinib, sunitinib malate, medroxyprogesterone acetate, hydroxyprogesterone caproate, megestrol acetate, raloxifene, bicalutamide, flutamide, nilutamide, megestrol acetate, CP-724714; TAK-165, HKI-272, erlotinib, lapatanib, canertinib, ABX-EGF antibody, erbitux, EKB-569, PKI-166, GW-572016, lonafarnib, 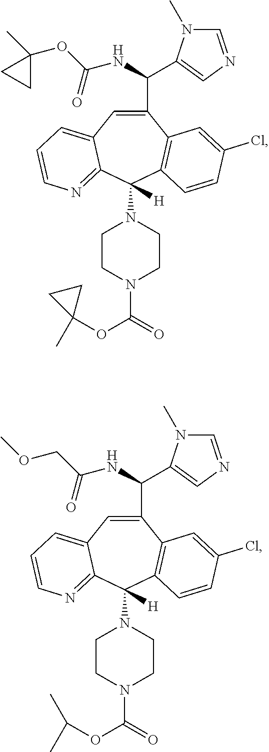
BMS-214662, tipifarnib; amifostine, NVP-LAQ824, suberoyl analide hydroxamic acid, valproic acid, trichostatin A, FK-228, SU11248, sorafenib, KRN951, aminoglutethimide, amsacrine, anagrelide, L-asparaginase, Bacillus Calmette-Guerin (BCG) vaccine, bleomycin, buserelin, busulfan, carboplatin, carmustine, chlorambucil, cisplatin, cladribine, clodronate, cyclophosphamide, cyproterone, cytarabine, dacarbazine, dactinomycin, daunorubicin, diethylstilbestrol, epirubicin, fludarabine, fludrocortisone, fluoxymesterone, flutamide, hydroxyurea, idarubicin, ifosfamide, imatinib, leucovorin, leuprolide, levamisole, lomustine, mechlorethamine, melphalan, 6-mercaptopurine, mesna, methotrexate, mitomycin, mitotane, mitoxantrone, nilutamide, octreotide, oxaliplatin, pamidronate, pentostatin, plicamycin, porfimer, procarbazine, raltitrexed, rituximab, streptozocin, teniposide, testosterone, thalidomide, thioguanine, thiotepa, tretinoin, vindesine, 13-cis-retinoic acid, phenylalanine mustard, uracil mustard, estramustine, altretamine, floxuridine, 5-deooxyuridine, cytosine arabinoside, 6-mercaptopurine, deoxycoformycin, calcitriol, valrubicin, mithramycin, vinblastine, vinorelbine, topotecan, razoxin, marimastat, COL-3, neovastat, BMS-275291, squalamine, endostatin, SU5416, SU6668, EMD121974, interleukin-12, IM862, angiostatin, vitaxin, droloxifene, idoxyfene, spironolactone, finasteride, cimitidine, trastuzumab, denileukin diftitox, gefitinib, bortezimib, paclitaxel, cremophor-free paclitaxel, docetaxel, epithilone B, BMS-247550, BMS-310705, droloxifene, 4-hydroxytamoxifen, pipendoxifene, ERA-923, arzoxifene, fulvestrant, acolbifene, lasofoxifene, idoxifene, TSE-424, HMR-3339, ZK186619, topotecan, PTK787/ZK 222584, VX-745, PD 184352, rapamycin, 40-O-(2-hydroxyethyl)-rapamycin, temsirolimus, AP-23573, RAD001, ABT-578, BC-210, LY294002, LY292223, LY292696, LY293684, LY293646, wortmannin, ZM336372, L-779,450, PEG-filgrastim, darbepoetin, 5-fluorouracil, erythropoietin, granulocyte colony-stimulating factor, zolendronate, prednisone, cetuximab, granulocyte macrophage colony-stimulating factor, histrelin, pegylated interferon alfa-2a, interferon alfa-2a, pegylated interferon alfa-2b, interferon alfa-2b, azacitidine, PEG-L-asparaginase, lenalidomide, gemtuzumab, hydrocortisone, interleukin-11, dexrazoxane, alemtuzumab, all-transretinoic acid, ketoconazole, interleukin-2, megestrol, immune globulin, nitrogen mustard, methylprednisolone, ibritgumomab tiuxetan, androgens, decitabine, hexamethylmelamine, bexarotene, tositumomab, arsenic trioxide, cortisone, editronate, mitotane, cyclosporine, liposomal daunorubicin, Edwina-asparaginase, strontium 89, casopitant, netupitant, an NK-1 receptor antagonists, palonosetron, aprepitant, diphenhydramine, hydroxyzine, metoclopramide, lorazepam, alprazolam, haloperidol, droperidol, dronabinol, dexamethasone, methylprednisolone, prochlorperazine, granisetron, ondansetron, dolasetron, tropisetron, pegfilgrastim, erythropoietin, epoetin alfa and darbepoetin alfa.

30. The antibody or fragment of claim 21 which is bound to IGF1R or a fragment thereof.

31. An isolated polypeptide comprising an amino acid sequence selected from the group consisting of SEQ ID NOs: 1-16, amino acids 20-144 of SEQ ID NO: 1, amino acids 23-129 of SEQ ID NO: 5, amino acids 20-146 of SEQ ID NO: 9 and amino acids 21-130 of SEQ ID NO: 13.

32. An isolated polynucleotide encoding the polypeptide of claim 31.

33. An isolated vector comprising the polynucleotide of claim 32.

34. An isolated host cell comprising the vector of claim 33.

35. The host cell of claim 34 which is bacterial or mammalian.

36. The host cell of claim 35 wherein the host cell is bacterial and is E. coli.

37. A method for treating or preventing a medical condition, in a subject, mediated by elevated expression or activity of insulin-like growth factor I receptor or elevated expression of IGF-1 or elevated expression of IGF-II comprising administering a therapeutically effective amount of an antibody or fragment of claim 1 to said subject.

38. The method of claim 37 wherein the medical condition is a member selected from the group consisting of osteosarcoma, rhabdomyosarcoma, neuroblastoma, any pediatric cancer, kidney cancer, leukemia, renal transitional cell cancer, Werner-Morrison syndrome, acromegaly, bladder cancer, Wilm's cancer, ovarian cancer, pancreatic cancer, benign prostatic hyperplasia, breast cancer, prostate cancer, bone cancer, lung cancer, gastric cancer, colorectal cancer, cervical cancer, synovial sarcoma, diarrhea associated with metastatic carcinoid, vasoactive intestinal peptide secreting tumors, gigantism, psoriasis, atherosclerosis, smooth muscle restenosis of blood vessels and inappropriate microvascular proliferation, head and neck cancer, squamous cell carcinoma, multiple myeloma, solitary plasmacytoma, renal cell cancer, retinoblastoma, germ cell tumors, hepatoblastoma, hepatocellular carcinoma, melanoma, rhabdoid tumor of the kidney, Ewing Sarcoma, chondrosarcoma, haemotological malignancy, chronic lymphoblastic leukemia, chronic myelomonocytic leukemia, acute lymphoblastic leukemia, acute lymphocytic leukemia, acute myelogenous leukemia, acute myeloblastic leukemia, chronic myeloblastic leukemia, Hodgekin's disease, non-Hodgekin's lymphoma, chronic lymphocytic leukemia, chronic myelogenous leukemia, myelodysplastic syndrome, hairy cell leukemia, mast cell leukemia, mast cell neoplasm, follicular lymphoma, diffuse large cell lymphoma, mantle cell lymphoma, Burkitt Lymphoma, mycosis fungoides, seary syndrome, cutaneous T-cell lymphoma, chronic myeloproliferative disorders, a cental nervous system tumor, brain cancer, glioblastoma, non-glioblastoma brain cancer, meningioma, pituitary adenoma, vestibular schwannoma, a primitive neuroectodermal tumor, medulloblastoma, astrocytoma, anaplastic astrocytoma, oligodendroglioma, ependymoma and choroid plexus papilloma, a myeloproliferative disorder, polycythemia vera, thrombocythemia, idiopathic myelfibrosis, soft tissue sarcoma, thyroid cancer, endometrial cancer, carcinoid cancer, germ cell tumors, liver cancer, gigantism, psoriasis, atherosclerosis, smooth muscle restenosis of blood vessels, inappropriate microvascular proliferation, acromegaly, gigantism, psoriasis, atherosclerosis, smooth muscle restenosis of blood vessels or inappropriate microvascular proliferation, Grave's disease, multiple sclerosis, systemic lupus erythematosus, Hashimoto's Thyroiditis, Myasthenia Gravis, auto-immune thyroiditis and Bechet's disease.

39. The method of claim 37 wherein the antibody or fragment is administered to the subject by a parenteral route.

40. The method of claim 37 wherein the antibody or fragment is administered to the subject in association with an additional chemotherapeutic agent or therapeutic procedure.

41. The method of claim 40 wherein the therapeutic procedure is radiation therapy or surgical tumorectomy.

42. A method for treating or preventing a medical condition, in a subject, mediated by elevated expression or activity of insulin-like growth factor I receptor or elevated expression of IGF-1 or elevated expression of IGF-II comprising administering a therapeutically effective amount of a composition of claim 9 to said subject.

43. The method of claim 37 wherein the antibody or fragment administered to the subject is a monoclonal antibody.

44. A method for treating or preventing a medical condition, in a subject, mediated by elevated expression or activity of insulin-like growth factor I receptor or elevated expression of IGF-1 or elevated expression of IGF-II comprising administering a therapeutically effective amount of an antibody or fragment of claim 11 to said subject.

45. The method of claim 44 wherein the medical condition is a member selected from the group consisting of osteosarcoma, rhabdomyosarcoma, neuroblastoma, any pediatric cancer, kidney cancer, leukemia, renal transitional cell cancer, Werner-Morrison syndrome, acromegaly, bladder cancer, Wilm's cancer, ovarian cancer, pancreatic cancer, benign prostatic hyperplasia, breast cancer, prostate cancer, bone cancer, lung cancer, gastric cancer, colorectal cancer, cervical cancer, synovial sarcoma, diarrhea associated with metastatic carcinoid, vasoactive intestinal peptide secreting tumors, gigantism, psoriasis, atherosclerosis, smooth muscle restenosis of blood vessels and inappropriate microvascular proliferation, head and neck cancer, squamous cell carcinoma, multiple myeloma, solitary plasmacytoma, renal cell cancer, retinoblastoma, germ cell tumors, hepatoblastoma, hepatocellular carcinoma, melanoma, rhabdoid tumor of the kidney, Ewing Sarcoma, chondrosarcoma, haemotological malignancy, chronic lymphoblastic leukemia, chronic myelomonocytic leukemia, acute lymphoblastic leukemia, acute lymphocytic leukemia, acute myelogenous leukemia, acute myeloblastic leukemia, chronic myeloblastic leukemia, Hodgekin's disease, non-Hodgekin's lymphoma, chronic lymphocytic leukemia, chronic myelogenous leukemia, myelodysplastic syndrome, hairy cell leukemia, mast cell leukemia, mast cell neoplasm, follicular lymphoma, diffuse large cell lymphoma, mantle cell lymphoma, Burkitt Lymphoma, mycosis fungoides, seary syndrome, cutaneous T-cell lymphoma, chronic myeloproliferative disorders, a cental nervous system tumor, brain cancer, glioblastoma, non-glioblastoma brain cancer, meningioma, pituitary adenoma, vestibular schwannoma, a primitive neuroectodermal tumor, medulloblastoma, astrocytoma, anaplastic astrocytoma, oligodendroglioma, ependymoma and choroid plexus papilloma, a myeloproliferative disorder, polycythemia vera, thrombocythemia, idiopathic myelfibrosis, soft tissue sarcoma, thyroid cancer, endometrial cancer, carcinoid cancer, germ cell tumors, liver cancer, gigantism, psoriasis, atherosclerosis, smooth muscle restenosis of blood vessels, inappropriate microvascular proliferation, acromegaly, gigantism, psoriasis, atherosclerosis, smooth muscle restenosis of blood vessels or inappropriate microvascular proliferation, Grave's disease, multiple sclerosis, systemic lupus erythematosus, Hashimoto's Thyroiditis, Myasthenia Gravis, auto-immune thyroiditis and Bechet's disease.

46. The method of claim 44 wherein the antibody or fragment is administered to the subject by a parenteral route.

47. The method of claim 44 wherein the antibody is administered to the subject in association with an additional therapeutic agent or therapeutic procedure.

48. The method of claim 47 wherein the therapeutic procedure is radiation therapy or surgical tumorectomy.

49. A method for treating or preventing a medical condition, in a subject, mediated by elevated expression or activity of insulin-like growth factor I receptor or elevated expression of IGF-1 or elevated expression of IGF-II comprising administering a therapeutically effective amount of a composition of claim 19 to said subject.

50. The method of claim 44 wherein the antibody or fragment administered to the subject is a monoclonal antibody.

51. A method for treating or preventing a medical condition, in a subject, mediated by elevated expression or activity of insulin-like growth factor I receptor or elevated expression of IGF-1 or elevated expression of IGF-II comprising administering a therapeutically effective amount of an antibody or fragment of claim 21 to said subject.

52. The method of claim 41 wherein the medical condition is a member selected from the group consisting of osteosarcoma, rhabdomyosarcoma, neuroblastoma, any pediatric cancer, kidney cancer, leukemia, renal transitional cell cancer, Werner-Morrison syndrome, acromegaly, bladder cancer, Wilm's cancer, ovarian cancer, pancreatic cancer, benign prostatic hyperplasia, breast cancer, prostate cancer, bone cancer, lung cancer, gastric cancer, colorectal cancer, cervical cancer, synovial sarcoma, diarrhea associated with metastatic carcinoid, vasoactive intestinal peptide secreting tumors, gigantism, psoriasis, atherosclerosis, smooth muscle restenosis of blood vessels and inappropriate microvascular proliferation, head and neck cancer, squamous cell carcinoma, multiple myeloma, solitary plasmacytoma, renal cell cancer, retinoblastoma, germ cell tumors, hepatoblastoma, hepatocellular carcinoma, melanoma, rhabdoid tumor of the kidney, Ewing Sarcoma, chondrosarcoma, haemotological malignancy, chronic lymphoblastic leukemia, chronic myelomonocytic leukemia, acute lymphoblastic leukemia, acute lymphocytic leukemia, acute myelogenous leukemia, acute myeloblastic leukemia, chronic myeloblastic leukemia, Hodgekin's disease, non-Hodgekin's lymphoma, chronic lymphocytic leukemia, chronic myelogenous leukemia, myelodysplastic syndrome, hairy cell leukemia, mast cell leukemia, mast cell neoplasm, follicular lymphoma, diffuse large cell lymphoma, mantle cell lymphoma, Burkitt Lymphoma, mycosis fungoides, seary syndrome, cutaneous T-cell lymphoma, chronic myeloproliferative disorders, a cental nervous system tumor, brain cancer, glioblastoma, non-glioblastoma brain cancer, meningioma, pituitary adenoma, vestibular schwannoma, a primitive neuroectodermal tumor, medulloblastoma, astrocytoma, anaplastic astrocytoma, oligodendroglioma, ependymoma and choroid plexus papilloma, a myeloproliferative disorder, polycythemia vera, thrombocythemia, idiopathic myelfibrosis, soft tissue sarcoma, thyroid cancer, endometrial cancer, carcinoid cancer, germ cell tumors, liver cancer, gigantism, psoriasis, atherosclerosis, smooth muscle restenosis of blood vessels, inappropriate microvascular proliferation, acromegaly, gigantism, psoriasis, atherosclerosis, smooth muscle restenosis of blood vessels or inappropriate microvascular proliferation, Grave's disease, multiple sclerosis, systemic lupus erythematosus, Hashimoto's Thyroiditis, Myasthenia Gravis, auto-immune thyroiditis and Bechet's disease.

53. The method of claim 51 wherein the antibody or fragment is administered to the subject by a parenteral route.

54. The method of claim 51 wherein the antibody or fragment is administered to the subject in association with an additional therapeutic agent or therapeutic procedure.

55. The method of claim 51 wherein the therapeutic procedure is radiation therapy or surgical tumorectomy.

56. A method for treating or preventing a medical condition, in a subject, mediated by elevated expression or activity of insulin-like growth factor I receptor or elevated expression of IGF-1 or elevated expression of IGF-II comprising administering a therapeutically effective amount of a composition of claim 29 to said subject.

57. The method of claim 51 wherein the antibody or fragment administered to the subject is a monoclonal antibody.

58. A method of detecting the presence or location of an IGF1R-expressing tumor in a subject, comprising
(i) administering the antibody or fragment of claim 1 to the subject; and
(ii) detecting binding of the antibody or fragment to said tumor, wherein said binding indicates the presence or location of said tumor.

59. An isolated antibody comprising a heavy chain immunoglobulin variable region comprising amino acids 20-144 of SEQ ID NO: 1 and a light chain immunoglobulin variable region comprising amino acids 23-129 of SEQ ID NO: 5.

60. An isolated antibody comprising a heavy chain immunoglobulin variable region comprising amino acids 20-146 of SEQ ID NO: 9 and a light chain immunoglobulin variable region comprising amino acids 21-130 of SEQ ID NO: 13.

61. An isolated hybridoma cell having American Type Culture Collection deposit number PTA-8428 or PTA-8429.

62. An isolated antibody produced by a hybridoma cell of claim 61.

63. A method for producing an isolated antibody comprising culturing a hybridoma cell of claim 61 in a culture medium under conditions suitable for expression of said antibody by said hybridoma and, optionally, purifying the antibody from the culture medium.

64. An antibody produced by the method of claim 63.
